SEQUENCE LISTING (181 sequences)

SEQ ID NO: 1 - protein, 122 aa, Oryctolagus cuniculus
QQQLEESGGGLVKPGGTLTLTCKASRIDFSTYYYICWVRQSPWKGLEWIACIYSGDGYTYYPSWAKGRFSISKTSSTTVTLQVTSLTAADTATYFCARSYGGSSASAYFTLWGPGTQVTVSS

SEQ ID NO: 2 - protein, 121 aa, Oryctolagus cuniculus
QQQLEESGGGLVKPGGTLTLTCKAGIDFSTYYYMCWVRQAPGKRPEWIACIFDGDGSTYYASWVNGRFTISKASSTTVTLRMTSLTAADTATYFCARSAGGSSSFGYFNLWGPGTLVTVSS

SEQ ID NO: 3 - protein, 123 aa, Oryctolagus cuniculus
QQHLEESGGGLVKPGGTLTLTCTASGIDFSSYYYMCWVRQAPGKGLEWIACIYTGDGSTYYASWVNGRFTISRSTSLNTVTLQLNGLAAADTATYFCARSYGASSAIGYFNLWGPGTLVTVSS

SEQ ID NO: 4 - protein, 118 aa, Oryctolagus cuniculus
QSVEESGGRLVMPGGSLTLTCTASGFSLSSYYMNWVRQAPGKGLEWIGVIYSTDITDYATWATGRFTISKTSTTVDLKMTSLTAADTATYFCARELSPTTSGYIAYLWGPGTLVTVSS

SEQ ID NO: 5 - protein, 115 aa, Oryctolagus cuniculus
QSVEESGGRLVTPGTPLTLSCKASGFDFSIYYMSWVRQAPGKGLEWIGVINSGGGVTWYASWVKGRFTISKTSTTVDLKITSPTTEDTATYFCARLGDSNHYDLWGPGTLVTVSS

SEQ ID NO: 6 - protein, 120 aa, Oryctolagus cuniculus
QEQLVESGGGLVKPEGSLTLTCTASGFSFSSDYSVCWVRQAPGKGLEWIGCIYTGSDITYYASWAKGRFTISKTSSTTVTLQMTSLTAADTATWFCARSWNGYGGYADLWGPGTLVTVSS

SEQ ID NO: 7 - protein, 121 aa, Oryctolagus cuniculus
QSLEESGGGLVKPGGTLTLTCTASGFSFSSSYWMCWVRQAPGKGLEWVACVYGGSDTTYYASWTKGRFTISKGSSTTVALQVTSLTAADTATYFCARDPLNYYYYGELNLWGPGTLVTVSS

SEQ ID NO: 8 - protein, 126 aa, Oryctolagus cuniculus
QQQLEESGGDLVQPEGSLTLTCKASGFDFSFNAMCWVRQAPGRGPEWIACIYNGNGSTYYASWAKGRFTISKTSSTSVTLQMTSLTAADTATYFCARGGYGLGYAGYGDVNYFNLWGPGTLVTVSS

SEQ ID NO: 9 - protein, 117 aa, Oryctolagus cuniculus
QSLEESGGRLVTPGTALTLTCTVSGFSLSSYAMGWVRQAPGKGLEYIGWTSPGDSAYYASWTKGRFTISRTSTTVELKITSPTTEDTATYFCARDAAYIGHWAFNLWGPGTLVTVSS

SEQ ID NO: 10 - protein, 125 aa, Oryctolagus cuniculus
QSVEESGGRLVTPGTPLTLTCTVSGFSLSSYAMSWVRQAPGKGLEWIGVISYSGRTYDASWAKGRFTISKTSTTVDLKITSPTTEDTATYFCARVGDSYGYAYATVIFTYHFNLWGPGTLVTVSS

SEQ ID NO: 11 - protein, 120 aa, Oryctolagus cuniculus
QEQVVEYGGDLVQPEGSLTLTCKASGLDFSSTYYMCWVRQAPGKGLEWIGCIYGGSSDSTYYASWAKGRFTISKTSPTTVTLQMTSLTAADTATHFCATYDYGAVGYDLWGPGTLVTVSS

SEQ ID NO: 12 - protein, 116 aa, synthetic construct
  REGION 1..116: note = IC17H VH
QVQLQQSGPQLVRPGASVNISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 13 - protein, 6 aa, synthetic construct
  REGION 1..6: note = DQN0223Hh HCDR1
TYYYIC

SEQ ID NO: 14 - protein, 5 aa, synthetic construct
  REGION 1..5: note = DQN0235He HCDR1
YYYMC

SEQ ID NO: 15 - protein, 6 aa, synthetic construct
  REGION 1..6: note = DQN0303Hh HCDR1
SYYYMC

SEQ ID NO: 16 - protein, 5 aa, synthetic construct
  REGION 1..5: note = DQN0333Hh HCDR1
SYYMN

SEQ ID NO: 17 - protein, 5 aa, synthetic construct
  REGION 1..5: note = DQN0282Hf HCDR1
IYYMS

SEQ ID NO: 18 - protein, 6 aa, synthetic construct
  REGION 1..6: note = DQN0356Hb HCDR1
SDYSVC

SEQ ID NO: 19 - protein, 6 aa, synthetic construct
  REGION 1..6: note = DQN0344Hx HCDR1
SSYWMC

SEQ ID NO: 20 - protein, 5 aa, synthetic construct
  REGION 1..5: note = DQN0334Hb HCDR1
FNAMC

SEQ ID NO: 21 - protein, 5 aa, synthetic construct
  REGION 1..5: note = DQN0139Hb HCDR1
SYAMG

SEQ ID NO: 22 - protein, 5 aa, synthetic construct
  REGION 1..5: note = DQN0177Ha HCDR1
SYAMS

SEQ ID NO: 23 - protein, 6 aa, synthetic construct
  REGION 1..6: note = DQN0089Hf HCDR1
STYYMC

SEQ ID NO: 24 - protein, 5 aa, synthetic construct
  REGION 1..5: note = IC17H LCDR1
SYWMH

SEQ ID NO: 25 - protein, 17 aa, synthetic construct
  REGION 1..17: note = DQN0223Hh HCDR2
CIYSGDGYTYYPSWAKG

SEQ ID NO: 26 - protein, 17 aa, synthetic construct
  REGION 1..17: note = DQN0235He HCDR2
CIFDGDGSTYYASWVNG

SEQ ID NO: 27 - protein, 17 aa, synthetic construct
  REGION 1..17: note = DQN0303Hh HCDR2
CIYTGDGSTYYASWVNG

SEQ ID NO: 28 - protein, 16 aa, synthetic construct
  REGION 1..16: note = DQN0333Hh HCDR2
VIYSTDITDYATWATG

SEQ ID NO: 29 - protein, 17 aa, synthetic construct
  REGION 1..17: note = DQN0282Hf HCDR2
VINSGGGVTWYASWVKG

SEQ ID NO: 30 - protein, 17 aa, synthetic construct
  REGION 1..17: note = DQN0356Hb HCDR2
CIYTGSDITYYASWAKG

SEQ ID NO: 31 - protein, 17 aa, synthetic construct
  REGION 1..17: note = DQN0344Hx HCDR2
CVYGGSDTTYYASWTKG

SEQ ID NO: 32 - protein, 17 aa, synthetic construct
  REGION 1..17: note = DQN0334Hb HCDR2
CIYNGNGSTYYASWAKG

SEQ ID NO: 33 - protein, 16 aa, synthetic construct
  REGION 1..16: note = DQN0139Hb HCDR2
WTSPGDSAYYASWTKG

SEQ ID NO: 34 - protein, 16 aa, synthetic construct
  REGION 1..16: note = DQN0177Ha HCDR2
VISYSGRTYDASWAKG

SEQ ID NO: 35 - protein, 18 aa, synthetic construct
  REGION 1..18: note = DQN0089Hf HCDR2
CIYGGSSDSTYYASWAKG

SEQ ID NO: 36 - protein, 17 aa, synthetic construct
  REGION 1..17: note = IC17H LCDR2
MIDPSYSETRLNQKFKD

SEQ ID NO: 37 - protein, 13 aa, synthetic construct
  REGION 1..13: note = DQN0223Hh HCDR3
SYGGSSASAYFTL

SEQ ID NO: 38 - protein, 13 aa, synthetic construct
  REGION 1..13: note = DQN0235He HCDR3
SAGGSSSFGYFNL

SEQ ID NO: 39 - protein, 13 aa, synthetic construct
  REGION 1..13: note = DQN0303Hh HCDR3
SYGASSAIGYFNL

SEQ ID NO: 40 - protein, 13 aa, synthetic construct
  REGION 1..13: note = DQN0333Hh HCDR3
ELSPTTSGYIAYL

SEQ ID NO: 41 - protein, 9 aa, synthetic construct
  REGION 1..9: note = DQN0282Hf HCDR3
LGDSNHYDL

SEQ ID NO: 42 - protein, 11 aa, synthetic construct
  REGION 1..11: note = DQN0356Hb HCDR3
SWNGYGGYADL

SEQ ID NO: 43 - protein, 13 aa, synthetic construct
  REGION 1..13: note = DQN0344Hx HCDR3
DPLNYYYYGELNL

SEQ ID NO: 44 - protein, 18 aa, synthetic construct
  REGION 1..18: note = DQN0334Hb HCDR3
GGYGLGYAGYGDVNYFNL

SEQ ID NO: 45 - protein, 12 aa, synthetic construct
  REGION 1..12: note = DQN0139Hb HCDR3
DAAYIGHWAFNL

SEQ ID NO: 46 - protein, 20 aa, synthetic construct
  REGION 1..20: note = DQN0177Ha HCDR3
VGDSYGYAYATVIFTYHFNL

SEQ ID NO: 47 - protein, 10 aa, synthetic construct
  REGION 1..10: note = DQN0089Hf HCDR3
YDYGAVGYDL

SEQ ID NO: 48 - protein, 7 aa, synthetic construct
  REGION 1..7: note = IC17H LCDR3
YGNYFDY

SEQ ID NO: 49 - protein, 111 aa, Oryctolagus cuniculus
AVVLTQTASPVSAPVGGTVTIKCQASQSIGSNLGWYQQKPGQPPKLLIYGASALASGVPSRFKGSGSGTQFTLTINDLECADAATYYCQGYYYSSSAEWHSFGGGTKVEIK

SEQ ID NO: 50 - protein, 112 aa, Oryctolagus cuniculus
AYDMTQTPSSVSAAVGGTVTIKCQASQSISSSYLAWYHQKPGQSPKLLIYSASTLASGVPSRFKGSGSGTEFTLTISDLACADAATYYCQSYYYFSSNAWHTFGGGTKVEIK

SEQ ID NO: 51 - protein, 111 aa, Oryctolagus cuniculus
DVVMTQTPSSKSAAVGGTVTIKCQASQNIYSNLAWYQQKPGQRPKLLIYEASTLASGVPSRFRGSGSGTEFTLTISDLECDDAATYYCQNYYYTSSNDFYTFGGGTKVEIK

SEQ ID NO: 52 - protein, 110 aa, Oryctolagus cuniculus
AYDMTQTPASVEVAVGGTVTIKCQASQSISSYLAWYQQKPGQPPKLLIYSASTLASGVPSRFKGSGSGTQFTLTVIDVQCDDAATYYCQQTYSGKNVINTFGGGTKVEIK

SEQ ID NO: 53 - protein, 108 aa, Oryctolagus cuniculus
AYDMTQTPSSVSAAVGGTVTIKCQASETISSYLAWYQQKPGQRPQLLIYDASNLASGVPSRFKGSGSGTEFTLTISDLECADAATYYCQTHYYITSTTFGGGTKVEIK

SEQ ID NO: 54 - protein, 110 aa, Oryctolagus cuniculus
AYDMAQTPASVEVAVGGTVTIKCQASQSIDNYLAWYQQKPGQPPKLLIYWASTLASGVSSRFKGSGSGTQFSLTISDLECADAATYYCQQYYSNSNVVNTFGGGTKVEIK

SEQ ID NO: 55 - protein, 108 aa, Oryctolagus cuniculus
AVVLTQTASPVSAAVGGTVTIRCQATENIYSGLAWYQQKPGQPPKVLIYYVSTLASGVPSRFKGSGSGTEYTLTISGVQCDDAATYYCQTYHDISNVTFGGGTKVEIK

SEQ ID NO: 56 - protein, 110 aa, Oryctolagus cuniculus
AVVLTQTASPVSAPVGGTVTIKCQASEDIYFLLAWYRQKPGQPPKLLIYGASTLASGVPSRFKGSGSGTEYTLTISDLECADAATYYCQSYWYGDSGPNTFGGGTKVEIK

SEQ ID NO: 57 - protein, 110 aa, Oryctolagus cuniculus
DVVMTQTPASVSAPVGGTVTINCQASESIYSNLAWYQQKPGQPPKLLIYGASTLESGVPSRVKGSGYGTQFTLTISDLECADAATYYCQQYYGSSSTAFTFGGGTKVEIK

SEQ ID NO: 58 - protein, 111 aa, Oryctolagus cuniculus
DPVLTQTPSSASEPVGGTVTIKCQASESISSSLAWYQQKPGQRPKLLIYDASKLASGVPSRFKGSGSGTEFTLTISGVQCDDAATYYCQHGLSSGSTNRCAFGGGTKVEIK

SEQ ID NO: 59 - protein, 111 aa, Oryctolagus cuniculus
EVVVTQTPASVEVAVGGTVTIKCQASQNISPYLSWYQQKPGQPPKLLIYKASTLASGVSSRFKGSGSGTQFTLTISDLECADAATYYCQNNYGVSINYGHTFGGGTKVEIK

SEQ ID NO: 60 - protein, 107 aa, synthetic construct
  REGION 1..107: note = IC17L VL
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVK

SEQ ID NO: 61 - protein, 11 aa, synthetic construct
  REGION 1..11: note = DQN0223Lh LCDR1
QASQSIGSNLG

SEQ ID NO: 62 - protein, 12 aa, synthetic construct
  REGION 1..12: note = DQN0235Le LCDR1
QASQSISSSYLA

SEQ ID NO: 63 - protein, 11 aa, synthetic construct
  REGION 1..11: note = DQN0303Lh LCDR1
QASQNIYSNLA

SEQ ID NO: 64 - protein, 11 aa, synthetic construct
  REGION 1..11: note = DQN0333Lh LCDR1
QASQSISSYLA

SEQ ID NO: 65 - protein, 11 aa, synthetic construct
  REGION 1..11: note = DQN0282Lf LCDR1
QASETISSYLA

SEQ ID NO: 66 - protein, 11 aa, synthetic construct
  REGION 1..11: note = DQN0356Lb LCDR1
QASQSIDNYLA

SEQ ID NO: 67 - protein, 11 aa, synthetic construct
  REGION 1..11: note = DQN0344Lx LCDR1
QATENIYSGLA

SEQ ID NO: 68 - protein, 11 aa, synthetic construct
  REGION 1..11: note = DQN0334Lb LCDR1
QASEDIYFLLA

SEQ ID NO: 69 - protein, 11 aa, synthetic construct
  REGION 1..11: note = DQN0139Lb LCDR1
QASESIYSNLA

SEQ ID NO: 70 - protein, 11 aa, synthetic construct
  REGION 1..11: note = DQN0177La LCDR1
QASESISSSLA

SEQ ID NO: 71 - protein, 11 aa, synthetic construct
  REGION 1..11: note = DQN0089Lf LCDR1
QASQNISPYLS

SEQ ID NO: 72 - protein, 11 aa, synthetic construct
  REGION 1..11: note = IC17L LCDR1
KASEDIYNRLA

SEQ ID NO: 73 - protein, 7 aa, synthetic construct
  REGION 1..7: note = DQN0223Lh LCDR2
GASALAS

SEQ ID NO: 74 - protein, 7 aa, synthetic construct
  REGION 1..7: note = DQN0235Le LCDR2
SASTLAS

SEQ ID NO: 75 - protein, 7 aa, synthetic construct
  REGION 1..7: note = DQN0303Lh LCDR2
EASTLAS

SEQ ID NO: 76 - protein, 7 aa, synthetic construct
  REGION 1..7: note = DQN0333Lh LCDR2
SASTLAS

SEQ ID NO: 77 - protein, 7 aa, synthetic construct
  REGION 1..7: note = DQN0282Lf LCDR2
DASNLAS

SEQ ID NO: 78 - protein, 7 aa, synthetic construct
  REGION 1..7: note = DQN0356Lb LCDR2
WASTLAS

SEQ ID NO: 79 - protein, 7 aa, synthetic construct
  REGION 1..7: note = DQN0344Lx LCDR2
YVSTLAS

SEQ ID NO: 80 - protein, 7 aa, synthetic construct
  REGION 1..7: note = DQN0334Lb LCDR2
GASTLAS

SEQ ID NO: 81 - protein, 7 aa, synthetic construct
  REGION 1..7: note = DQN0139Lb LCDR2
GASTLES

SEQ ID NO: 82 - protein, 7 aa, synthetic construct
  REGION 1..7: note = DQN0177La LCDR2
DASKLAS

SEQ ID NO: 83 - protein, 7 aa, synthetic construct
  REGION 1..7: note = DQN0089Lf LCDR2
KASTLAS

SEQ ID NO: 84 - protein, 7 aa, synthetic construct
  REGION 1..7: note = IC17L LCDR2
GATSLET

SEQ ID NO: 85 - protein, 13 aa, synthetic construct
  REGION 1..13: note = DQN0223Lh LCDR3
QGYYYSSSAEWHS

SEQ ID NO: 86 - protein, 13 aa, synthetic construct
  REGION 1..13: note = DQN0235Le LCDR3
QSYYYFSSNAWHT

SEQ ID NO: 87 - protein, 13 aa, synthetic construct
  REGION 1..13: note = DQN0303Lh LCDR3
QNYYYTSSNDFYT

SEQ ID NO: 88 - protein, 12 aa, synthetic construct
  REGION 1..12: note = DQN0333Lh LCDR3
QQTYSGKNVINT

SEQ ID NO: 89 - protein, 10 aa, synthetic construct
  REGION 1..10: note = DQN0282Lf LCDR3
QTHYYITSTT

SEQ ID NO: 90 - protein, 12 aa, synthetic construct
  REGION 1..12: note = DQN0356Lb LCDR3
QQYYSNSNVVNT

SEQ ID NO: 91 - protein, 10 aa, synthetic construct
  REGION 1..10: note = DQN0344Lx LCDR3
QTYHDISNVT

SEQ ID NO: 92 - protein, 12 aa, synthetic construct
  REGION 1..12: note = DQN0334Lb LCDR3
QSYWYGDSGPNT

SEQ ID NO: 93 - protein, 12 aa, synthetic construct
  REGION 1..12: note = DQN0139Lb LCDR3
QQYYGSSSTAFT

SEQ ID NO: 94 - protein, 13 aa, synthetic construct
  REGION 1..13: note = DQN0177La LCDR3
QHGLSSGSTNRCA

SEQ ID NO: 95 - protein, 13 aa, synthetic construct
  REGION 1..13: note = DQN0089Lf LCDR3
QNNYGVSINYGHT

SEQ ID NO: 96 - protein, 9 aa, synthetic construct
  REGION 1..9: note = IC17L LCDR3
QQYWSTPYT

SEQ ID NO: 97 - protein, 328 aa, synthetic construct
  REGION 1..328: note = F1332m CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 98 - protein, 107 aa, synthetic construct
  REGION 1..107: note = k0MC CL
RTVAAPSVFIFPPSDEQLKSGTASVVCLLNNFYPREAKVQWKVDNALQSGNSQESVTEQDSKDCTYSLSSTLTLSKADYEKHKVYACEVTHQGLSSPVTKSFNRGEC

SEQ ID NO: 99 - protein, 19 aa, synthetic construct
  REGION 1..19: note = CAMPATH-1H signal sequence
MGWSCIILFLVATATGVHS

SEQ ID NO: 100 - protein, 4 aa, synthetic construct
  REGION 1..4: note = GGGG linker
GGGG

SEQ ID NO: 101 - protein, 33 aa, synthetic construct
  REGION 1..33: note = 33mer gliadin peptide sequence
LQLQPFPQPELPYPQPELPYPQPELPYPQPQPF

SEQ ID NO: 102 - protein, 5 aa, synthetic construct
  REGION 1..5: note = GGGGG linker
GGGGG

SEQ ID NO: 103 - protein, 29 aa, synthetic construct
  REGION 1..29: note = An artificially synthesized sequence
KLPKPPKPVSKMRMATPLLMQELPMGALP

SEQ ID NO: 104 - protein, 12 aa, synthetic construct
  REGION 1..12: note = SSADLVPRGGGG linker
SSADLVPRGGGG

SEQ ID NO: 105 - protein, 18 aa, synthetic construct
  REGION 1..18: note = gliadin peptide sequence
QQYPSGEGSFQPSQENPQ

SEQ ID NO: 106 - protein, 13 aa, synthetic construct
  REGION 1..13: note = SSADLVPRGGGGG linker
SSADLVPRGGGGG

SEQ ID NO: 107 - protein, 20 aa, synthetic construct
  REGION 1..20: note = An artificially synthesized sequence
ATGSNCPPHIENFSDIDMGE

SEQ ID NO: 108 - protein, 18 aa, synthetic construct
  REGION 1..18: note = CAMPATH-1H signal sequence
MGWSCIILFLVATATGVH

SEQ ID NO: 109 - protein, 18 aa, synthetic construct
  REGION 1..18: note = signal sequence, rat serum albumin
MKWVTFLLLLFISGSAFS

SEQ ID NO: 110 - DNA, 825 bp, Homo sapiens
  source 1..825: mol_type = unassigned DNA
gaattccaccatgggctggagctgcatcatcctgtttctggtggcaaccgcaacaggagtgcacagcaagaccacacagcccccttccatggactgcgcagagggaagggcagcaaacctgccctgtaatcacagcaccatctccggcaacgagtacgtgtattggtacagacagatccactctcagggccctcagtatatcatccacggcctgaagaacaatgagacaaatgagatggcctccctgatcatcaccgaggaccggaagagctccacactgatcctgccacacgccaccctgagagacacagccgtgtactattgcatcgtgctgggaggagcagatggactgacctttggcaagggcacacacctgatcatccagccctatatccagaacccagatcccgccgtgtaccagctgagggactccaagtctagcgataagagcgtgtgcctgttcaccgactttgattctcagacaaacgtgagccagagcaaggacagcgacgtgtacatcaccgacaagacagtgctggatatgagatccatggacttcaagtccaactctgccgtggcctggtctaataagagcgatttcgcctgcgccaacgcctttaacaattccatcatccctgaggataccttctttccttctccagagtcctcttgtgacgtgaagctggtggagaagagcttcgagaccgatacaaacctgaattttcagaacctgtccgtgatcggcttcaggatcctgctgctgaaggtggccggcttcaatctgctgatgaccctgcgcctgtggagctcctagtaagcggccgc

SEQ ID NO: 111 - DNA, 945 bp, Homo sapiens
  source 1..945: mol_type = unassigned DNA
gaattccaccatgggctggtcctgcatcatcctgttcctggtggcaaccgcaacaggagtgcactctggcgtgtcccagtctcccagcaacaaggtgaccgagaagggcaaggacgtggagctgcggtgtgatcctatcagcggccacacagccctgtactggtatagacagtccctgggccagggcctggagttcctgatctactttcagggcaatagcgcccctgacaagtctggcctgccaagcgatcggttctccgccgagagaaccggaggatccgtgtctaccctgacaatccagcggacacagcaggaggactccgccgtgtacctgtgcgccagctccttcaggtttaccgatacacagtattttggaccaggaaccaggctgacagtgctggaggacctgaagaacgtgttcccccctgaggtggccgtgtttgagccatctgaggccgagatcagccacacccagaaggccacactggtgtgcctggccaccggcttctaccccgatcacgtggagctgtcctggtgggtgaacggcaaggaggtgcactctggcgtgtgcacagacccacagcccctgaaggagcagcctgccctgaatgattctcggtacgccctgtctagccggctgagagtgagcgccaccttttggcagaacccaaggaatcacttccgctgccaggtgcagttttatggcctgagcgagaatgacgagtggacccaggatagggccaagcctgtgacacagatcgtgtccgccgaggcatggggaagggcagactgtggcttcaccagcgtgtcctatcagcagggcgtgctgtctgccaccatcctgtacgagatcctgctgggcaaggccacactgtatgccgtgctggtgtccgccctggtgctgatggccatggtgaagagaaaggatttttagtaagcggccgc

SEQ ID NO: 112 - protein, 254 aa, Homo sapiens
MILNKALMLGALALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 113 - protein, 254 aa, Homo sapiens
MILNKALMLGALALTTVMSPCGGEDIVADHVASYGVNLYQSYGPSGQFTHEFDGDEEFYVDLERKETVWKLPLFHRLRFDPQFALTNIAVLKHNLNILIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPAPMSELTETVVCALGLSVGLVGIVVGTVLIIRGLRSVGASRHQGPL

SEQ ID NO: 114 - protein, 255 aa, Homo sapiens [sequence omitted]

SEQ ID NO: 115 - protein, 255 aa, Homo sapiens
MILNKALLLGALALTTVMSPCGGEDIVADHVASCGVNLYQFYGPSGQYTHEFDGDEQFYVDLERKETAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHWEPEIPAPMSELTETVVCALGLSVGLMGIVVGTVFIIQGLRSVGASRHQGPL

SEQ ID NO: 116 - protein, 255 aa, Homo sapiens [sequence omitted]

SEQ ID NO: 117 - protein, 255 aa, Homo sapiens; identical to SEQ ID NO: 115

SEQ ID NO: 118 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSDDEIYDCKVEHWGLDEPLLKHWEPEIPTPMSELTETVVCALGLSVGLVGIVVGTVLIIRGLRSVGASRHQGPL

SEQ ID NO: 119 - protein, 254 aa, Homo sapiens
MILNKALMLGTLALTTVMSPCGGEDIVADHVASYGVNLYQSYGPSGQYTHEFDGDEQFYVDLGRKETVWCLPVLRQFRFDPQFALTNIAVLKHNLNSLIKRSNSTAATNEVPEVTVFSKSPVTLGQPNILICLVDNIFPPVVNITWLSNGHSVTEGVSETSFLSKSDHSFFKISYLTLLPSAEESYDCKVEHWGLDKPLLKHWEPEIPAPMSELTETVVCALGLSVGLVGIVVGTVFIIRGLRSVGASRHQGPL

SEQ ID NO: 120 - protein, 255 aa, Homo sapiens
MILNKALMLGALALTTVMSPCGGEDIVADHVASYGVNLYQSYGPSGQYSHEFDGDEEFYVDLERKETVWQLPLFRRFRRFDPQFALTNIAVLKHNLNIVIKRSNSTAATNEVPEVTVFSKSPVTLGQPNTLICLVDNIFPPVVNITWLSNGHSVTEGVSETSFLSKSDHSFFKISYLTFLPSADEIYDCKVEHWGLDEPLLKHWEPEIPTPMSELTETVVCALGLSVGLVGIVVGTVLIIRGLRSVGASRHQGPL

SEQ ID NO: 121 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNDQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 122 - protein, 261 aa, Homo sapiens
MSWKKALRIPGGLRAATVTLMLSMLSTPVAEGRDSPEDFVYQFKGMCYFTNGTERVRLVSRSIYNREEIVRFDSDVGEFRAVTLLGLPAAEYWNSQKDILERKRAAVDRVCRHNYQLELRTTLQRRVEPTVTISPSRTEALNHHNLLVCSVTDFYPAQIKVRWFRNGQEETAGVVSTPLIRNGDWTFQILVMLEMTPQRGDVYTCHVEHPSLQSPITVEWRAQSESAQSKMLSGIGGFVLGLIFLGLGLIIHHRSQKGLLH

SEQ ID NO: 123 - protein, 261 aa, Homo sapiens [sequence omitted]

SEQ ID NO: 124 - protein, 261 aa, Homo sapiens
MSWKKALRIPGDLRVATVTLMLAMLSSLLAEGRDSPEDFVFQFKGMCYFTNGTERVRLVTRYIYNREEYARFDSDVGVYRAVTPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGLLH

SEQ ID NO: 125 - protein, 261 aa, Homo sapiens [sequence omitted]

SEQ ID NO: 126 - protein, 261 aa, Homo sapiens [sequence omitted]

SEQ ID NO: 127 - protein, 261 aa, Homo sapiens
MSWKKALRIPGGLRAATVTLMLAMLSTPVAEGRDSPEDFVYQFKAMCYFTNGTERVRYVTRYIYNREEYARFDSDVEVYRAVTPLGPPDAEYWNSQKEVLERTRAELDTVCRHNYQLELRTTLQRRVEPTVTISPSRTEALNHHNLLVCSVTDFYPAQIKVRWFRNDQEETTGVVSTPLIRNGDWTFQILVMLEMTPQHGDVYTCHVEHPSLQNPITVEWRAQSESAQSKMLSGIGGFVLGLIFLGLGLIIHHRSQKGLLH

SEQ ID NO: 128 - protein, 261 aa, Homo sapiens; identical to SEQ ID NO: 127

SEQ ID NO: 129 - protein, 261 aa, Homo sapiens [sequence omitted]

SEQ ID NO: 130 - protein, 13 aa, synthetic construct
  REGION 1..13: note = alpha 1 gliadin peptide sequence
QPFPQPELPYPGS

SEQ ID NO: 131 - protein, 14 aa, synthetic construct
  REGION 1..14: note = alpha 1b gliadin peptide sequence
QLPYPQPELPYPGS

SEQ ID NO: 132 - protein, 13 aa, synthetic construct
  REGION 1..13: note = alpha 2 gliadin peptide sequence
APQPELPYPQPGS

SEQ ID NO: 133 - protein, 14 aa, synthetic construct
  REGION 1..14: note = omega 1 gliadin peptide sequence
QQPFPQPEQPFPGS

SEQ ID NO: 134 - protein, 14 aa, synthetic construct
  REGION 1..14: note = omega 2 gliadin peptide sequence
QFPQPEQPFPWQGS

SEQ ID NO: 135 - protein, 17 aa, synthetic construct
  REGION 1..17: note = secalin 1 peptide sequence
QPEQPFPQPEQPFPQGS

SEQ ID NO: 136 - protein, 17 aa, synthetic construct
  REGION 1..17: note = secalin 2 peptide sequence
QQPFPQPEQPFPQSQGS

SEQ ID NO: 137 - protein, 9 aa, synthetic construct
  REGION 1..9: note = salmonella peptide sequence
MMAWRMMRY

SEQ ID NO: 138 - protein, 14 aa, synthetic construct
  REGION 1..14: note = Mycobacterium bovis peptide sequence
KPLLIIAEDVEGEY

SEQ ID NO: 139 - protein, 15 aa, synthetic construct
  REGION 1..15: note = Hepatitis B virus peptide sequence
PDRVHFASPLHVAWR

SEQ ID NO: 140 - protein, 323 aa, Oryctolagus cuniculus
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 141 - protein, 104 aa, Oryctolagus cuniculus
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 142 - protein, 121 aa, Oryctolagus cuniculus
QSLEESGGGLVKPGGTLTLTCTASGFSFSSAYYMCWVRQAPGKGLEWIACIYVGSGSTYYASWAQGRCTISKASSTTVTLQMTSLTAADTATYFCARDPLIYYYYGELNLWGPGTLVTVSS

SEQ ID NO: 143 - protein, 122 aa, Oryctolagus cuniculus
QSLEESGGGLVKPGASLTLTCKASGFSFSSGYYMCWVRQAPGKGLEWIACIYAGSPDLTYYATWAKGRLTISTTSSTTVTLQLTSLTAADTATYFCARDPGNWGGYDDVRLWGPGTLVTVSS

SEQ ID NO: 144 - protein, 119 aa, Oryctolagus cuniculus
QSLEESGGDLVKPGASLALTCTASGFSLINNYYMCWVRQAPGKGLEWIACIYAGLGGWTYYASWAKGRFTISKTSSTTVTLQMSSLTVADAATYFCARAATTYYHFNLWGPGTLVTVSS

SEQ ID NO: 145 - protein, 121 aa, Oryctolagus cuniculus
QSLEESGGGLVKPGGPLTLTCTASGFSFSSAYYMCWVRQAPGKGLEWIACIYVGSGSTYYTSWAKGRFTISKVSSTTMTLQMTSLTAADTATYFCARDPLIYYYYGELNLWGPGTLVTVSS

SEQ ID NO: 146 - protein, 6 aa, synthetic construct
  REGION 1..6: note = DQN0225dd HCDR1
SAYYMC

SEQ ID NO: 147 - protein, 6 aa, synthetic construct
  REGION 1..6: note = DQN0271hh HCDR1
SGYYMC

SEQ ID NO: 148 - protein, 6 aa, synthetic construct
  REGION 1..6: note = DQN0324hh HCDR1
NNYYMC

SEQ ID NO: 149 - protein, 6 aa, synthetic construct
  REGION 1..6: note = DQN0370hh HCDR1
SAYYMC

SEQ ID NO: 150 - protein, 17 aa, synthetic construct
  REGION 1..17: note = DQN0225dd HCDR2
CIYVGSGSTYYASWAQG

SEQ ID NO: 151 - protein, 18 aa, synthetic construct
  REGION 1..18: note = DQN0271hh HCDR2
CIYAGSPDLTYYATWAKG

SEQ ID NO: 152 - protein, 18 aa, synthetic construct
  REGION 1..18: note = DQN0324hh HCDR2
CIYAGLGGWTYYASWAKG

SEQ ID NO: 153 - protein, 17 aa, synthetic construct
  REGION 1..17: note = DQN0370hh HCDR2
CIYVGSGSTYYTSWAKG

SEQ ID NO: 154 - protein, 13 aa, synthetic construct
  REGION 1..13: note = DQN0225dd HCDR3
DPLIYYYYGELNL

SEQ ID NO: 155 - protein, 13 aa, synthetic construct
  REGION 1..13: note = DQN0271hh HCDR3
DPGNWGGYDDVRL

SEQ ID NO: 156 - protein, 10 aa, synthetic construct
  REGION 1..10: note = DQN0324hh HCDR3
AATTYYHFNL

SEQ ID NO: 157 - protein, 13 aa, synthetic construct
  REGION 1..13: note = DQN0370hh HCDR3
DPLIYYYYGELNL

SEQ ID NO: 158 - protein, 108 aa, Oryctolagus cuniculus
AVVLTQTASPVSAPVGGTVTIKCQASEDISSNLAWYQQKPGQRPKVLIYSASTLASGVSSQFKGSGSGTQFTLTISDLECADAATYYCQSYYDISSVTFGGGTKVEIK

SEQ ID NO: 159 - protein, 109 aa, Oryctolagus cuniculus
AQVLTQTPSPVSAAVGGTVTISCQSSQSIYSNYLSWFQQKTGQPPKLLIYRASTLASGVPSRFKGSGSGTEFTLTISGVECDDAATYYCQGYYSGAIWTFGGGTKVEIK

SEQ ID NO: 160 - protein, 111 aa, Oryctolagus cuniculus
AVVLTQTESPVSAAVGDTVTIKCQASQSIGNALAWYQQKPGQPPKLLIYDASTLASGVPSRFKGSGSGTQFTLTISDLECADAATYYCQSYDYGSSADTYTFGGGTKVEIK

SEQ ID NO: 161 - protein, 108 aa, Oryctolagus cuniculus
AVVLTQTASPVSAPVGGTVTIKCQASEDIYSNLAWYQQKPGQRPKVLIYSASTLASGVSSRFKGSRSGTEYTLSITDLGCADAATYYCQAYYDIGGVTFGGGTKVEIK

SEQ ID NO: 162 - protein, 11 aa, synthetic construct
  REGION 1..11: note = DQN0225dd LCDR1
QASEDISSNLA

SEQ ID NO: 163 - protein, 12 aa, synthetic construct
  REGION 1..12: note = DQN0271hh LCDR1
QSSQSIYSNYLS

SEQ ID NO: 164 - protein, 11 aa, synthetic construct
  REGION 1..11: note = DQN0324hh LCDR1
QASQSIGNALA

SEQ ID NO: 165 - protein, 11 aa, synthetic construct
  REGION 1..11: note = DQN0370hh LCDR1
QASEDIYSNLA

SEQ ID NO: 166 - protein, 7 aa, synthetic construct
  REGION 1..7: note = DQN0225dd LCDR2
SASTLAS

SEQ ID NO: 167 - protein, 7 aa, synthetic construct
  REGION 1..7: note = DQN0271hh LCDR2
RASTLAS

SEQ ID NO: 168 - protein, 7 aa, synthetic construct
  REGION 1..7: note = DQN0324hh LCDR2
DASTLAS

SEQ ID NO: 169 - protein, 7 aa, synthetic construct
  REGION 1..7: note = DQN0370hh LCDR2
SASTLAS

SEQ ID NO: 170 - protein, 10 aa, synthetic construct
  REGION 1..10: note = DQN0225dd LCDR3
QSYYDISSVT

SEQ ID NO: 171 - protein, 10 aa, synthetic construct
  REGION 1..10: note = DQN0271hh LCDR3
QGYYSGAIWT

SEQ ID NO: 172 - protein, 13 aa, synthetic construct
  REGION 1..13: note = DQN0324hh LCDR3
QSYDYGSSADTYT

SEQ ID NO: 173 - protein, 10 aa, synthetic construct
  REGION 1..10: note = DQN0370hh LCDR3
QAYYDIGGVT

SEQ ID NO: 174 - protein, 114 aa, Oryctolagus cuniculus
QSLEESGGRLVTPGGSLTLTCTVSGIDLSNYAVGWVRQAPGKGLEYIGIIYASGSAYYASWAKGRFTISKTSSTTVDLKVTSLTTEDTATYFCARGYGRAFRIWGPGTLVTVSS

SEQ ID NO: 175 - protein, 5 aa, Oryctolagus cuniculus
NYAVG

SEQ ID NO: 176 - protein, 16 aa, Oryctolagus cuniculus
IIYASGSAYYASWAKG

SEQ ID NO: 177 - protein, 8 aa, Oryctolagus cuniculus
GYGRAFRI

SEQ ID NO: 178 - protein, 109 aa, Oryctolagus cuniculus
AVLTQTPSPVSAAVGGTVTISCQASQSVYNNNFLSWYQQKPGQRPKLLIYDASTLESGVPSRFSGSGSGTQFTLTISSVQCDDAATYYCLGGYDDHDNAFGGGTEVVVK

SEQ ID NO: 179 - protein, 13 aa, Oryctolagus cuniculus
QASQSVYNNNFLS

SEQ ID NO: 180 - protein, 7 aa, Oryctolagus cuniculus
DASTLES

SEQ ID NO: 181 - protein, 10 aa, Oryctolagus cuniculus
LGGYDDHDNA

(19) United States
(12) Patent Application Publication
(10) Pub. No.: US 2023/0118024 A1
(43) Pub. Date: Apr. 20, 2023

(54) ANTI-HLA-DQ2.5 ANTIBODY

(71) Applicant: Chugai Seiyaku Kabushiki Kaisha, Tokyo (JP)

(72) Inventors: Yuu Okura, Singapore (SG); Noriyuki Takahashi, Singapore (SG); Takashi Tsushima, Singapore (SG); Zulkarnain Harfuddin, Singapore (SG); Akihiko Mizoroki, Kanagawa (JP)

(73) Assignee: Chugai Seiyaku Kabushiki Kaisha, Tokyo (JP)

(21) Appl. No.: 17/974,949

(22) Filed: Oct. 27, 2022

Related U.S. Application Data
(63) Continuation of application No. 16/652,707, filed on Apr. 1, 2020.

(30) Foreign Application Priority Data
Oct. 3, 2017 (JP) 2017-193341

(51) Int. Cl. C07K 16/28

(52) U.S. Cl. CPC C07K 16/2833; C07K 2317/33; C07K 2317/76; C07K 2317/92

(57) ABSTRACT

The antibodies of the present invention have specific binding activity to HLA-DQ2.5 and may have binding activity to HLA-DQ2.2 and/or HLA-DQ7.5, but substantially no binding activity to HLA-DQ8, HLA-DQ5.1, HLA-DQ6.3, HLA-DQ7.3, HLA-DR, HLA-DP, or a complex of the invariant chain (CD74) and HLA-DQ2.5. The antibodies bind to HLA-DQ2.5 in the presence of a gluten peptide such as gliadin, i.e., bind to HLA-DQ2.5 forming a complex with the gluten peptide. The antibodies have neutralizing activity against the binding between HLA-DQ2.5 and TCR, and thus block the interaction between HLA-DQ2.5 and an HLA-DQ2.5-restricted CD4+ T cell. The antibodies do not undergo rapid internalization mediated by the invariant chain.

### CROSS-REFERENCE TO RELATED APPLICATIONS

This application is a continuation of U.S. application Ser. No. 16/652,707, filed on Apr. 1, 2020, which is the National Stage of International Application No. PCT/JP2018/037078, filed on Oct. 3, 2018, which claims the benefit of Japanese Application No. 2017-193341, filed on Oct. 3, 2017.

### SEQUENCE LISTING

This application contains a Sequence Listing that has been submitted electronically as an XML file named C1-A1714P-USD1sq.xml. The XML file, created on Oct. 24, 2022, is 214,071 bytes in size. The material in the XML file is hereby incorporated by reference in its entirety.

### TECHNICAL FIELD

The present invention relates to anti-HLA-DQ2.5 antibodies

### BACKGROUND ART

Celiac (coeliac) disease is an autoimmune disorder in which the ingestion of gluten causes damage to the small intestine in genetically-sensitive patients (NPL 1 to 5). About 1% of the Western population, i.e., 8 million people in the United States and the European Union are thought to suffer from celiac disease; however, no remarkable therapeutic advances have been achieved since the disease was recognized in 1940s.

Human leukemia antigens (HLAs) belonging to Major Histocompatibility Complex (MHC) class II include HLA-DR, HLA-DP and HLA-DQ molecules such as the HLA-DQ2.5 isoform (hereinafter referred to as “HLA-DQ2.5”), which form heterodimers composed of alpha and beta chains on the cell surface. A majority (>90%) of the celiac disease patients have an HLA-DQ2.5 haplotype allele (NPL 6). The isoform is thought to have stronger affinity towards a gluten peptide. As with other isoforms, HLA-DQ2.5 presents processed antigens derived from exogenous sources to a T cell receptor (TCR) on T cells. As a result of digestion of gluten-rich food such as bread in celiac disease patients, immunogenic gluten peptides such as gliadin peptides are formed (NPL 2). The peptides are transported through the small intestine epithelium into lamina propria and deamidated by tissue transglutaminase such as transglutaminase 2 (TG2). The deamidated gliadin peptides are processed by antigen-presenting cells (APCs) which load them on HLA-DQ2.5. The loaded peptides are presented to HLA-DQ2.5-restricted T cells, and activate innate and adaptive immune responses. This causes inflammatory injury of the small intestinal mucosa and symptoms including various types of gastrointestinal disturbance, nutritional deficiencies, and systemic symptoms. It is reported that an anti-HLA DQ neutralizing antibody inhibits activation of T cells from celiac patients. (NPL7)

The currently practicable treatment of celiac disease is lifelong adherence to a gluten-free diet (GFD). However, in reality, it is difficult to completely eliminate gluten exposure even with GFD. The tolerable gluten dose for these patients is only about 10 to 50 mg/day (NPL 11). Cross contamination can widely occur in GFD production, and a trace amount of gluten can cause celiac disease symptoms even in patients with good compliance to GFD. In the presence of such a risk of unintentional gluten exposure, there is a need for adjunctive therapy to GFD.

### CITATION LIST

### Non Patent Literature

[NPL 1] N Engl J Med 2007; 357:1731-1743
[NPL 2] J Biomed Sci. 2012; 19(1): 88
[NPL 3] N Engl J Med 2003; 348:2517-2524
[NPL 4] Gut 2003; 52:960-965
[NPL 5] Dig Dis Sci 2004; 49:1479-1484
[NPL 6] Gastroenterology 2011; 141:610-620
[NPL 7] Gut 2005; 54:1217-1223
[NPL 8] Gastroenterology 2014; 146:1649-58
[NPL 9] Nutrients 2013 Oct. 5(10): 3975-3992
[NPL 10] J Clin Invest. 2007; 117(1):41-49
[NPL 11] Am J Clin Nutr 2007; 85: 160-6

### SUMMARY OF INVENTION

### Technical Problem

Under the above-mentioned circumstances with the need for adjunctive therapy, the present invention provides anti-HLA-DQ2.5 antibodies.

### Solution to Problem

In certain embodiments, the anti-HLA-DQ2.5 antibody of the present invention (hereinafter also referred to as “the antibody of the present invention”) has binding activity to HLA-DQ2.5 and substantially no binding activity to HLA-DQ8.

In certain embodiments, the antibody of the present invention has binding activity to HLA-DQ2.5 in the form of a complex with a gluten peptide (an HLA-DQ2.5/gluten peptide complex).

In certain embodiments, the gluten peptide is at least one, two, three, four, five, six, seven or all of the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide and a secalin 2 peptide.

In certain embodiments, the gluten peptide is a 33mer gliadin peptide.

In certain embodiments, the antibody of the present invention blocks the interaction between the HLA-DQ2.5/gluten peptide complex and an HLA-DQ2.5/gluten peptide-restricted CD4+ T cell.

In certain embodiments, the antibody of the present invention has substantially no binding activity to HLA-DQ5.1, HLA-DQ6.3, or HLA-DQ7.3.

In certain embodiments, the antibody of the present invention has substantially no binding activity to HLA-DR or HLA-DP.

In certain embodiments, the antibody of the present invention has substantially no binding activity to HLA-DQ2.5 in the form of a complex with an invariant chain (an HLA-DQ2.5/invariant chain complex).

In certain embodiments, the antibody of the present invention has binding activity to HLA-DQ2.2 and substantially no binding activity to HLA-DQ7.5.

In certain embodiments, the antibody of the present invention has binding activity to HLA-DQ7.5 and substantially no binding activity to HLA-DQ2.2.

In certain embodiments, the antibody of the present invention has substantially no binding activity to HLA-DQ2.2 or HLA-DQ7.5.

In certain embodiments, the antibody of the present invention has enhanced binding activity to HLA-DQ2.5 in the form of a complex with a gluten peptide.

In certain embodiments, the antibody of the present invention has stronger binding activity to HLA-DQ2.5 in the form of a complex with at least one, two, three, four, five, six, seven or all of the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide and a secalin 2 peptide (an HLA-DQ2.5/33mer gliadin peptide complex, an HLA-DQ2.5/alpha 1 gliadin peptide complex, an HLA-DQ2.5/alpha 1b gliadin peptide complex, an HLA-DQ2.5/alpha 2 gliadin peptide complex, an HLA-DQ2.5/omega 1 gliadin peptide complex, an HLA-DQ2.5/omega 2 gliadin peptide complex, an HLA-DQ2.5/secalin 1 peptide complex and an HLA-DQ2.5/secalin 2 peptide complex) than to HLA-DQ2.5 in the form of a complex with at least one, two, three, four or all of the group consisting of a CLIP peptide, a salmonella peptide, a Mycobacterium bovis peptide, a Hepatitis B virus peptide and a HLA-DQ2.5 positive PBMC-B cell (an HLA-DQ2.5/CLIP peptide complex, an HLA-DQ2.5/salmonella peptide complex, an HLA-DQ2.5/Mycobacterium bovis peptide complex, an HLA-DQ2.5/Hepatitis B virus peptide complex and an HLA-DQ2.5 positive PBMC-B cell).

In certain embodiments, the antibody of the present invention has stronger binding activity to HLA-DQ2.5 in the form of a complex with a 33mer gliadin peptide (an HLA-DQ2.5/33mer gliadin peptide complex) than to HLA-DQ2.5 in the form of a complex with a CLIP peptide (an HLA-DQ2.5/CLIP peptide complex).

In certain embodiments, the antibody of the present invention has neutralizing activity against the binding between gliadin-bound HLA-DQ2.5 and D2 TCR or S2 TCR.

In certain embodiments, the antibody of the present invention does not undergo cell internalization with the invariant chain (i.e., invariant chain-mediated rapid cell internalization).

In certain embodiments, the antibody of the present invention is a humanized antibody.

In certain embodiments, the antibody of the present invention has specific heavy-chain complementarity determining regions (HCDRs).

In certain embodiments, the antibody of the present invention has specific light-chain complementarity determining regions (LCDRs).

In certain embodiments, the present invention provides an antibody that binds to the same HLA-DQ2.5 epitope bound by the antibody that has the specific HCDRs and LCDRs.

In certain embodiments, the present invention provides an antibody that competes for HLA-DQ2.5 binding with the antibody that has the specific HCDRs and LCDRs.

In certain embodiments, the present invention provides an anti-HLA-DQ2.5 antibody which has binding activity to the beta chain of HLA-DQ2.5 and blocks the interaction between an HLA-DQ2.5/gluten peptide complex and an HLA-DQ2.5/gluten peptide-restricted CD4+ T cell.

In certain embodiments, the present invention provides an anti-HLA-DQ2.5 antibody which has binding activity to the alpha chain of HLA-DQ2.5 and blocks the interaction between an HLA-DQ2.5/gluten peptide complex and an HLA-DQ2.5/gluten peptide-restricted CD4+ T cell.

In certain embodiments, the present invention provides an antibody which has binding activity to HLA-DQ2.5 in the form of a complex with at least one, two, three, four, five, six, seven or all of the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide and a secalin 2 peptide and substantially no binding activity to HLA-DQ2.5 in the form of a complex with at least one, two, three, four or all of the group consisting of a CLIP peptide, a salmonella peptide, a Mycobacterium bovis peptide, a Hepatitis B virus peptide and a HLA-DQ2.5 positive PBMC-B cell and blocks the interaction between an HLA-DQ2.5/gluten peptide complex and an HLA-DQ2.5/gluten peptide-restricted CD4+ T cell.

In certain embodiments, the present invention provides an antibody which has binding activity to HLA-DQ2.5 in the form of a complex with a 33mer gliadin peptide and substantially no binding activity to HLA-DQ2.5 in the form of a complex with a CLIP peptide and blocks the interaction between an HLA-DQ2.5/gluten peptide complex and an HLA-DQ2.5/gluten peptide-restricted CD4+ T cell.

In certain embodiments, the present invention provides a method of screening for an anti-HLA-DQ2.5 antibody, which comprises testing whether the antibody has binding activity to an antigen(s) of interest and selecting the antibody that has binding activity to the antigen(s) of interest; testing whether the antibody has specific binding activity to an antigen(s) of no interest and selecting the antibody that has no specific binding activity to the antigen(s) of no interest.

In certain embodiments, the above method further comprises: testing whether the antibody has neutralizing activity against the binding between HLA-DQ2.5 and TCR; and selecting the antibody that has the neutralizing activity.

In certain embodiments, the above method further comprises: testing whether the antibody binds to HLA-DQ2.5 in the presence of a gluten peptide such as gliadin; and selecting the antibody that binds to HLA-DQ2.5 in the presence of the gluten peptide.

More specifically, the present invention provides the following.

[1] An anti-HLA-DQ2.5 antibody which has binding activity to HLA-DQ2.5 and substantially no binding activity to HLA-DQ8.

[2] The antibody of [1], wherein the antibody has binding activity to HLA-DQ2.5 in the form of a complex with a gluten peptide (an HLA-DQ2.5/gluten peptide complex).

[3] The antibody of [2], wherein the gluten peptide is at least one, two, three, four, five, six, seven or all of the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide and a secalin 2 peptide.

[4] The antibody of [2], wherein the antibody blocks the interaction between the HLA-DQ2.5/gluten peptide complex and an HLA-DQ2.5/gluten peptide-restricted CD4+ T cell.

[5] The antibody of any one of [1] to [4], wherein the antibody has substantially no binding activity to HLA-DQ5.1, HLA-DQ6.3, or HLA-DQ7.3.

[6] The antibody of any one of [1] to [5], wherein the antibody has substantially no binding activity to HLA-DR or HLA-DP.

[7] The antibody of any one of [1] to [6], wherein the antibody has substantially no binding activity to HLA-DQ2.5 in the form of a complex with an invariant chain (an HLA-DQ2.5/invariant chain complex).

[8] The antibody of any one of [1] to [7], wherein the antibody has binding activity to HLA-DQ2.2 and substantially no binding activity to HLA-DQ7.5.

[9] The antibody of any one of [1] to [7], wherein the antibody has binding activity to HLA-DQ7.5 and substantially no binding activity to HLA-DQ2.2.

[10] The antibody of any one of [1] to [7], wherein the antibody has substantially no binding activity to HLA-DQ2.2 or HLA-DQ7.5.

[11] The antibody of [10], wherein the antibody has enhanced binding activity to HLA-DQ2.5 in the form of a complex with a gluten peptide.

[12] The antibody of [11], wherein the antibody has stronger binding activity to HLA-DQ2.5 in the form of a complex with at least one, two, three, four, five, six, seven or all of the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide and a secalin 2 peptide (an HLA-DQ2.5/33mer gliadin peptide complex, an HLA-DQ2.5/alpha 1 gliadin peptide complex, an HLA-DQ2.5/alpha 1b gliadin peptide complex, an HLA-DQ2.5/alpha 2 gliadin peptide complex, an HLA-DQ2.5/omega 1 gliadin peptide complex, an HLA-DQ2.5/omega 2 gliadin peptide complex, an HLA-DQ2.5/secalin 1 peptide complex and an HLA-DQ2.5/secalin 2 peptide complex) than to HLA-DQ2.5 in the form of a complex with at least one, two, three, four or all of the group consisting of a CLIP peptide, a salmonella peptide, a Mycobacterium bovis peptide, a Hepatitis B virus peptide and a HLA-DQ2.5 positive PBMC-B cell (an HLA-DQ2.5/CLIP peptide complex, an HLA-DQ2.5/salmonella peptide complex, an HLA-DQ2.5/Mycobacterium bovis peptide complex, an HLA-DQ2.5/Hepatitis B virus peptide complex and an HLA-DQ2.5 positive PBMC-B cell).

[13] The antibody of any one of [1] to [7], which is any one of (1) to (14) below:

(1) an antibody comprising the HCDR1 sequence of SEQ ID NO: 13, the HCDR2 sequence of SEQ ID NO: 25, the HCDR3 sequence of SEQ ID NO: 37, the LCDR1 sequence of SEQ ID NO: 61, the LCDR2 sequence of SEQ ID NO: 73, and the LCDR3 sequence of SEQ ID NO: 85;

(2) an antibody comprising the HCDR1 sequence of SEQ ID NO: 14, the HCDR2 sequence of SEQ ID NO: 26, the HCDR3 sequence of SEQ ID NO: 38, the LCDR1 sequence of SEQ ID NO: 62, the LCDR2 sequence of SEQ ID NO: 74, and the LCDR3 sequence of SEQ ID NO: 86;

(3) an antibody comprising the HCDR1 sequence of SEQ ID NO: 15, the HCDR2 sequence of SEQ ID NO: 27, the HCDR3 sequence of SEQ ID NO: 39, the LCDR1 sequence of SEQ ID NO: 63, the LCDR2 sequence of SEQ ID NO: 75, and the LCDR3 sequence of SEQ ID NO: 87;

(4) an antibody comprising the HCDR1 sequence of SEQ ID NO: 16, the HCDR2 sequence of SEQ ID NO: 28, the HCDR3 sequence of SEQ ID NO: 40, the LCDR1 sequence of SEQ ID NO: 64, the LCDR2 sequence of SEQ ID NO: 76, and the LCDR3 sequence of SEQ ID NO: 88;

(5) an antibody comprising the HCDR1 sequence of SEQ ID NO: 17, the HCDR2 sequence of SEQ ID NO: 29, the HCDR3 sequence of SEQ ID NO: 41, the LCDR1 sequence of SEQ ID NO: 65, the LCDR2 sequence of SEQ ID NO: 77, and the LCDR3 sequence of SEQ ID NO: 89;

(6) an antibody comprising the HCDR1 sequence of SEQ ID NO: 18, the HCDR2 sequence of SEQ ID NO: 30, the HCDR3 sequence of SEQ ID NO: 42, the LCDR1 sequence of SEQ ID NO: 66, the LCDR2 sequence of SEQ ID NO: 78, and the LCDR3 sequence of SEQ ID NO: 90;

(7) an antibody comprising the HCDR1 sequence of SEQ ID NO: 19, the HCDR2 sequence of SEQ ID NO: 31, the HCDR3 sequence of SEQ ID NO: 43, the LCDR1 sequence of SEQ ID NO: 67, the LCDR2 sequence of SEQ ID NO: 79, and the LCDR3 sequence of SEQ ID NO: 91;

(8) an antibody comprising the HCDR1 sequence of SEQ ID NO: 20, the HCDR2 sequence of SEQ ID NO: 32, the HCDR3 sequence of SEQ ID NO: 44, the LCDR1 sequence of SEQ ID NO: 68, the LCDR2 sequence of SEQ ID NO: 80, and the LCDR3 sequence of SEQ ID NO: 92;

(9) an antibody comprising the HCDR1 sequence of SEQ ID NO: 146, the HCDR2 sequence of SEQ ID NO: 150, the HCDR3 sequence of SEQ ID NO: 154, the LCDR1 sequence of SEQ ID NO: 162, the LCDR2 sequence of SEQ ID NO: 166, and the LCDR3 sequence of SEQ ID NO: 170;

(10) an antibody comprising the HCDR1 sequence of SEQ ID NO: 147, the HCDR2 sequence of SEQ ID NO: 151, the HCDR3 sequence of SEQ ID NO: 155, the LCDR1 sequence of SEQ ID NO: 163, the LCDR2 sequence of SEQ ID NO: 167, and the LCDR3 sequence of SEQ ID NO: 17192;

(11) an antibody comprising the HCDR1 sequence of SEQ ID NO: 148, the HCDR2 sequence of SEQ ID NO: 152, the HCDR3 sequence of SEQ ID NO: 156, the LCDR1 sequence of SEQ ID NO: 164, the LCDR2 sequence of SEQ ID NO: 168, and the LCDR3 sequence of SEQ ID NO: 172;

(12) an antibody comprising the HCDR1 sequence of SEQ ID NO: 149, the HCDR2 sequence of SEQ ID NO: 153, the HCDR3 sequence of SEQ ID NO: 157, the LCDR1 sequence of SEQ ID NO: 165, the LCDR2 sequence of SEQ ID NO: 169, and the LCDR3 sequence of SEQ ID NO: 173;

(13) an antibody that binds to the same HLA-DQ2.5 epitope bound by the antibody of any one of (1) to (12);

(14) an antibody that competes with the antibody of any one of (1) to (12) for binding to HLA-DQ2.5 or a complex of a gluten peptide and HLA-DQ2.5.

[14] An anti-HLA-DQ2.5 antibody which has binding activity to the beta chain of HLA-DQ2.5 and blocks the interaction between an HLA-DQ2.5/gluten peptide complex and an HLA-DQ2.5/gluten peptide-restricted CD4+ T cell.

[15] An anti-HLA-DQ2.5 antibody which has binding activity to the alpha chain of HLA-DQ2.5 and blocks the interaction between an HLA-DQ2.5/gluten peptide complex and an HLA-DQ2.5/gluten peptide-restricted CD4+ T cell.

[16] An antibody which has binding activity to HLA-DQ2.5 in the form of a complex with at least one, two, three, four, five, six, seven or all of the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide and a secalin 2 peptide and substantially no binding activity to HLA-DQ2.5 in the form of a complex with at least one, two, three, four or all of the group consisting of a CLIP peptide, a salmonella peptide, a Mycobacterium bovis peptide, a Hepatitis B virus peptide and a HLA-DQ2.5 positive PBMC-B cell and blocks the interaction between an HLA-DQ2.5/gluten peptide complex and an HLA-DQ2.5/gluten peptide-restricted CD4+ T cell.

[17] An anti-HLA-DQ2.5 antibody which has binding activity to HLA-DQ2.5 and substantially no binding activity to HLA-DQ8.

[18] The antibody of [17], wherein the antibody has binding activity to HLA-DQ2.5 in the form of a complex with a gluten peptide (an HLA-DQ2.5/gluten peptide complex).

[19] The antibody of [18], wherein the gluten peptide is a 33mer gliadin peptide.

[20] The antibody of [18], wherein the antibody blocks the interaction between the HLA-DQ2.5/gluten peptide complex and an HLA-DQ2.5/gluten peptide-restricted CD4+ T cell.

[21] The antibody of any one of [17] to [20], wherein the antibody has substantially no binding activity to HLA-DQ5.1, HLA-DQ6.3, or HLA-DQ7.3.

[22] The antibody of any one of [17] to [21], wherein the antibody has substantially no binding activity to HLA-DR or HLA-DP.

[23] The antibody of any one of [17] to [22], wherein the antibody has substantially no binding activity to HLA-DQ2.5 in the form of a complex with an invariant chain (an HLA-DQ2.5/invariant chain complex).

[24] The antibody of any one of [17] to [23], wherein the antibody has binding activity to HLA-DQ2.2 and substantially no binding activity to HLA-DQ7.5.

[25] The antibody of any one of [17] to [23], wherein the antibody has binding activity to HLA-DQ7.5 and substantially no binding activity to HLA-DQ2.2.

[26] The antibody of any one of [17] to [23], wherein the antibody has substantially no binding activity to HLA-DQ2.2 or HLA-DQ7.5.

[27] The antibody of [26], wherein the antibody has enhanced binding activity to HLA-DQ2.5 in the form of a complex with a gluten peptide.

[28] The antibody of [27], wherein the antibody has stronger binding activity to HLA-DQ2.5 in the form of a complex with a 33mer gliadin peptide (an HLA-DQ2.5/33mer gliadin peptide complex) than to HLA-DQ2.5 in the form of a complex with a CLIP peptide (an HLA-DQ2.5/CLIP peptide complex).

[29] The antibody of any one of [17] to [23], which is any one of (1) to (10) below:

(1) an antibody comprising the HCDR1 sequence of SEQ ID NO: 13, the HCDR2 sequence of SEQ ID NO: 25, the HCDR3 sequence of SEQ ID NO: 37, the LCDR1 sequence of SEQ ID NO: 61, the LCDR2 sequence of SEQ ID NO: 73, and the LCDR3 sequence of SEQ ID NO: 85;

(2) an antibody comprising the HCDR1 sequence of SEQ ID NO: 14, the HCDR2 sequence of SEQ ID NO: 26, the HCDR3 sequence of SEQ ID NO: 38, the LCDR1 sequence of SEQ ID NO: 62, the LCDR2 sequence of SEQ ID NO: 74, and the LCDR3 sequence of SEQ ID NO: 86;

(3) an antibody comprising the HCDR1 sequence of SEQ ID NO: 15, the HCDR2 sequence of SEQ ID NO: 27, the HCDR3 sequence of SEQ ID NO: 39, the LCDR1 sequence of SEQ ID NO: 63, the LCDR2 sequence of SEQ ID NO: 75, and the LCDR3 sequence of SEQ ID NO: 87;

(4) an antibody comprising the HCDR1 sequence of SEQ ID NO: 16, the HCDR2 sequence of SEQ ID NO: 28, the HCDR3 sequence of SEQ ID NO: 40, the LCDR1 sequence of SEQ ID NO: 64, the LCDR2 sequence of SEQ ID NO: 76, and the LCDR3 sequence of SEQ ID NO: 88;

(5) an antibody comprising the HCDR1 sequence of SEQ ID NO: 17, the HCDR2 sequence of SEQ ID NO: 29, the HCDR3 sequence of SEQ ID NO: 41, the LCDR1 sequence of SEQ ID NO: 65, the LCDR2 sequence of SEQ ID NO: 77, and the LCDR3 sequence of SEQ ID NO: 89;

(6) an antibody comprising the HCDR1 sequence of SEQ ID NO: 18, the HCDR2 sequence of SEQ ID NO: 30, the HCDR3 sequence of SEQ ID NO: 42, the LCDR1 sequence of SEQ ID NO: 66, the LCDR2 sequence of SEQ ID NO: 78, and the LCDR3 sequence of SEQ ID NO: 90;

(7) an antibody comprising the HCDR1 sequence of SEQ ID NO: 19, the HCDR2 sequence of SEQ ID NO: 31, the HCDR3 sequence of SEQ ID NO: 43, the LCDR1 sequence of SEQ ID NO: 67, the LCDR2 sequence of SEQ ID NO: 79, and the LCDR3 sequence of SEQ ID NO: 91;

(8) an antibody comprising the HCDR1 sequence of SEQ ID NO: 20, the HCDR2 sequence of SEQ ID NO: 32, the HCDR3 sequence of SEQ ID NO: 44, the LCDR1 sequence of SEQ ID NO: 68, the LCDR2 sequence of SEQ ID NO: 80, and the LCDR3 sequence of SEQ ID NO: 92;

(9) an antibody that binds to the same HLA-DQ2.5 epitope bound by the antibody of any one of (1) to (8);

(10) an antibody that competes with the antibody of any one of (1) to (8) for binding to HLA-DQ2.5 or a complex of a gluten peptide and HLA-DQ2.5.

[30] An anti-HLA-DQ2.5 antibody which has binding activity to the beta chain of HLA-DQ2.5 and blocks the interaction between an HLA-DQ2.5/gluten peptide complex and an HLA-DQ2.5/gluten peptide-restricted CD4+ T cell.

[31] An anti-HLA-DQ2.5 antibody which has binding activity to the alpha chain of HLA-DQ2.5 and blocks the interaction between an HLA-DQ2.5/gluten peptide complex and an HLA-DQ2.5/gluten peptide-restricted CD4+ T cell.

[32] An antibody which has binding activity to HLA-DQ2.5 in the form of a complex with a 33mer gliadin peptide and substantially no binding activity to HLA-DQ2.5 in the form of a complex with a CLIP peptide and blocks the interaction between an HLA-DQ2.5/gluten peptide complex and an HLA-DQ2.5/gluten peptide-restricted CD4+ T cell.

### BRIEF DESCRIPTION OF DRAWINGS

FIG. 1 shows FACS results of the binding of the antibodies to HLA-DQ2.5/33mer gliadin peptide (Example 4.1).

FIG. 2 shows FACS results of the binding of the antibodies to HLA-DQ2.5/CLIP peptide (Example 4.1).

FIG. 3 shows FACS results of the binding of the antibodies to HLA-DQ2.5 (Example 4.1).

FIG. 4 shows FACS results of the binding of the antibodies to HLA-DQ2.2 (Example 4.1).

FIG. 5 shows FACS results of the binding of the antibodies to HLA-DQ7.5 (Example 4.1).

FIG. 6 shows FACS results of the binding of the antibodies to HLA-DP (Example 4.2).

FIG. 7 shows FACS results of the binding of the antibodies to HLA-DR (Example 4.2).

FIG. 8 shows FACS results of the binding of the antibodies to HLA-DQ8 (Example 4.2).

FIG. 9 shows FACS results of the binding of the antibodies to HLA-DQ5.1 (Example 4.2).

FIG. 10 shows FACS results of the binding of the antibodies to HLA-DQ6.3 (Example 4.2).

FIG. 11 shows FACS results of the binding of the antibodies to HLA-DQ7.3 (Example 4.2).

FIG. 12 shows the AlphaLISA-based neutralizing activity of the antibodies against the binding between an HLA-DQ2.5/33mer gliadin peptide complex and D2 TCR (Example 4.4).

FIG. 13 shows the beads-based neutralizing activity of the antibodies against the binding between an HLA-DQ2.5/33mer gliadin peptide complex and S2 TCR (Example 4.4).

FIG. 14 shows the binding of the antibodies to the HLA-DQ2.5/invariant chain complex (Example 4.5).

FIG. 15 shows the cell-based neutralizing activity of the antibodies against the binding between an HLA-DQ2.5/33mer gliadin peptide complex and D2 TCR (Example 4.6).

FIG. 16 shows FACS results of the binding of the antibodies to HLA-DQ2.5 (Example 7).

FIG. 17 shows FACS results of the binding of the antibodies to HLA-DQ2.5/CLIP peptide (Example 7).

FIG. 18 shows FACS results of the binding of the antibodies to HLA-DQ2.5/33mer gliadin peptide (Example 7).

FIG. 19 shows FACS results of the binding of the antibodies to HLA-DQ2.5/alpha 1 gliadin peptide (Example 7).

FIG. 20 shows FACS results of the binding of the antibodies to HLA-DQ2.5/alpha 1b gliadin peptide (Example 7).

FIG. 21 shows FACS results of the binding of the antibodies to HLA-DQ2.5/alpha 2 gliadin peptide (Example 7).

FIG. 22 shows FACS results of the binding of the antibodies to HLA-DQ2.5/omega 1 gliadin peptide (Example 7).

FIG. 23 shows FACS results of the binding of the antibodies to HLA-DQ2.5/omega 2 gliadin peptide (Example 7).

FIG. 24 shows FACS results of the binding of the antibodies to HLA-DQ2.5/secalin 1 peptide (Example 7).

FIG. 25 shows FACS results of the binding of the antibodies to HLA-DQ2.5/secalin 2 peptide (Example 7).

FIG. 26 shows FACS results of the binding of the antibodies to HLA-DQ2.5/salmonella peptide (Example 7).

FIG. 27 shows FACS results of the binding of the antibodies to HLA-DQ2.5/Mycobacterium bovis peptide (Example 7).

FIG. 28 shows FACS results of the binding of the antibodies to HLA-DQ2.5/Hepatitis B virus peptide (Example 7).

FIG. 29 shows FACS results of the binding of the antibodies to HLA-DQ2.5+PBMC B cell (Example 8).

FIG. 30 shows summary of FACS results of the binding of the antibodies to HLA-DQ2.5/several peptides (Example 8).

FIG. 31 shows FACS results of the binding of the antibodies to HLA-DQ2.2 (Example 9).

FIG. 32 shows FACS results of the binding of the antibodies to HLA-DQ7.5 (Example 9).

FIG. 33 shows FACS results of the binding of the antibodies to HLA-DQ8 (Example 10).

FIG. 34 shows FACS results of the binding of the antibodies to HLA-DQ5.1 (Example 10).

FIG. 35 shows FACS results of the binding of the antibodies to HLA-DQ6.3 (Example 10).

FIG. 36 shows FACS results of the binding of the antibodies to HLA-DQ7.3 (Example 10).

FIG. 37 shows FACS results of the binding of the antibodies to HLA-DR (Example 10).

FIG. 38 shows FACS results of the binding of the antibodies to HLA-DP (Example 10).

FIG. 39 shows the cell-based neutralizing activity of the antibodies against the binding between an HLA-DQ2.5/33mer gliadin peptide complex and D2 TCR (Example 11).

FIG. 40 shows the AlphaLISA-based neutralizing activity of the antibodies against the binding between an HLA-DQ2.5/33mer gliadin peptide complex and D2 TCR (Example 12).

FIG. 41 shows the beads-based neutralizing activity of the antibodies against the binding between an HLA-DQ2.5/33mer gliadin peptide complex and S2 TCR (Example 13).

FIG. 42 shows the binding of the antibodies to the HLA-DQ2.5/invariant chain complex (Example 15).

FIG. 43 shows the ELISA result of the primary screening. The identified single hit (positive) B-cell clone could bind to IgG1 delta-GK and IgG4 delta-GK specifically but not to IgG1 delta-K and IgG4 delta-K. An anti-keyhole limpet hemocyanin (KLH) rabbit monoclonal antibody was used as an isotype control.

FIG. 44 shows the ELISA result of the secondary screening. The identified single hit (positive) B-cell clone could bind to IgG1 delta-GK and IgG4 delta-GK specifically but not to IgG1 delta-GK-amide and IgG4 delta-GK-amide. An anti-KLH rabbit monoclonal antibody was used as an isotype control.

FIG. 45 shows the ELISA result of the purified monoclonal antibody. YG55 could bind to IgG1 delta-GK and IgG4 delta-GK specifically but not to IgG1 delta-GK-amide and IgG4 delta-GK-amide. An anti-KLH rabbit monoclonal antibody was used as an isotype control.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-HLA-DQ2.5 antibody” and “an antibody which has binding activity to HLA-DQ2.5” refer to an antibody that is capable of binding HLA-DQ2.5 (herein referred to as “HLA-DQ2.5”) with sufficient affinity such that the antibody is useful as a diagnostic and/or therapeutic agent in targeting HLA-DQ2.5. In one embodiment, the extent of binding of an anti-HLA-DQ2.5 antibody to an unrelated, non-HLA-DQ2.5 protein is less than about 10% of the binding of the antibody to HLA-DQ2.5 as measured, e.g., by a radioimmunoassay (RIA). In certain embodiments, an antibody which has binding activity to HLA-DQ2.5 has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸M to 10⁻¹³M, e.g., from 10⁻⁹M to 10⁻¹³M). In certain embodiments, an anti-HLA-DQ2.5 antibody binds to an epitope of HLA-DQ2.5 that is conserved among HLA-DQ2.5 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Autoimmune disease” refers to a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, celiac disease, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobulinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

The term “celiac (coeliac) disease” refers to a hereditary autoimmune disease caused by damages in the small intestine upon ingestion of gluten contained in food. Symptoms of celiac disease include, but not limited to, gastrointestinal disturbance such as abdominal pain, diarrhea, and gastroesophageal reflux, vitamin deficiency, mineral deficiency, central nervous system (CNS) symptoms such as fatigue and anxiety depression, bone symptoms such as osteomalacia and osteoporosis, skin symptoms such as skin inflammation, blood symptoms such as anemia and lymphocytopenia, and other symptoms such as infertility, hypogonadism, and children's failure to thrive and short stature.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

Herein, the term “gluten” collectively refers to a composite of storage proteins called prolamins found in wheat and other related grains. In the gut lumen, gluten is degraded into so-called gluten peptides. Gluten peptides include, but are not limited to, gliadin from wheat, hordein from barley, and secalin from rye, and avenin from oat.

The phrase “having substantially no binding activity”, as used herein, refers to activity of an antibody to bind to an antigen of no interest at a level of binding that includes non-specific or background binding but does not include specific binding. In other words, such an antibody “has no specific/significant binding activity” towards the antigen of no interest. The specificity can be measured by any methods mentioned in this specification or known in the art. The above-mentioned level of non-specific or background binding may be zero, or may not be zero but near zero, or may be very low enough to be technically neglected by those skilled in the art. For example, when a skilled person cannot detect or observe any significant (or relatively strong) signal for binding between the antibody and the antigen of no interest in a suitable binding assay, it can be said that the antibody has “substantially no binding activity” or “no specific/significant binding activity” towards the antigen of no interest. Alternatively, “have substantially no binding activity” or “have no specific/significant binding activity” can be rephrased as “do/does not specifically/significantly/substantially bind” (to the antigen of no interest). Sometimes, the phrase “having no binding activity” has substantially the same meaning as the phrase “having substantially no binding activity” or “having no specific/significant binding activity” in the art.

Herein, “HLA-DR/DP” means “HLA-DR and HLA-DP” or “HLA-DR or HLA-DP”. These HLAs are MHC class II molecules encoded by the corresponding haplotype alleles on the WIC class II locus in human. “HLA-DQ” collectively refers to HLA-DQ isoforms including HLA-DQ2.5, HLA-DQ2.2, HLA-DQ7.5, HLA-DQ5.1, HLA-DQ6.3, HLA-DQ7.3, and HLA-DQ8. In the present invention, “HLA-DQ molecules other than HLA-DQ2.5, HLA-DQ2.2, or HLA-DQ7.5” include, but are not limited to, HLA-DQ molecules of known subtypes (isoforms) such as HLA-DQ2.3, HLA-DQ4.3, HLA-DQ4.4, HLA-DQ5.1, HLA-DQ5.2, HLA-DQ5.3, HLA-DQ5.4, HLA-DQ6.1, HLA-DQ6.2, HLA-DQ6.3, HLA-DQ6.4, HLA-DQ6.9, HLA-DQ7.2, HLA-DQ7.3, HLA-DQ7.4, HLA-DQ7.5, HLA-DQ7.6, HLA-DQ8, HLA-DQ9.2, and HLA-DQ9.3. Similarly, “HLA-DR (DP)” refers to HLA-DR (DP) isoforms.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

In one embodiment, HVR residues comprise those identified in the specification.]] Unless otherwise indicated, HVR residues and other residues in the variable domain (e.g., FR residues) are numbered herein according to Kabat et al., supra.

An “immunoconjugate” is an antibody conjugated to one or more heterologous molecule(s).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

Herein, “(the) invariant chain” refers to a protein encoded by a gene for human CD74 (GenBank accession No. NM_001025159). Thus, “invariant chain” is also called “CD74” or “CD74/invariant chain”. The invariant chain forms a complex with an MHC class II molecule such as HLA-DQ2.5, and this complex can be located on the membrane of the endoplasmic reticulum or the endosome, or the plasma membrane of an MHC class II-expressing cell. The term “invariant chain (76-295)” refers to the partial peptide consisting of the amino acid residues from positions 76 to 295 of the invariant chain according to GenBank accession No. NM_001025159. CLIP (Class II-associated invariant chain peptide) is a portion of the invariant chain (CD74). In the present invention, a CLIP peptide (for example, SEQ ID NO: 103) may be used together with a suitable HLA-DQ molecule such as HLA-DQ2.5, HLA-DQ2.2, and HLA-DQ7.5 when evaluating the binding of the anti-HLA-DQ2.5 antibodies to these HLA-DQ molecules. Meanwhile, for HLA-DQ5.1, a DBY peptide (for example, SEQ ID NO: 107) may be used for this purpose. This peptide is a portion of the DBY protein which is an HLA-DQ5-restricted histocompatibility antigen.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-HLA-DQ2.5 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa and lambda, based on the amino acid sequence of its constant domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared.

The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “HLA-DQ2.5,” as used herein, refers to any native HLA-DQ2.5 from any vertebrate source, including mammals such as primates (e.g. humans) and rodents (e.g., mice and rats), unless otherwise indicated. The term encompasses “full-length” unprocessed HLA-DQ2.5 as well as any form of HLA-DQ2.5 that results from processing in the cell. The term also encompasses naturally occurring variants of HLA-DQ2.5, e.g., splice variants or allelic variants. The amino acid sequence of exemplary HLA-DQ2.5 is publicly available in Research Collaboratory for Structural Bioinformatics (RCSB) Protein Data Bank (PDB) accession code 4OZG.

Herein, “TCR” means “T-cell receptor” which is a membrane protein located on the surface of T cells (such as HLA-DQ2.5-restricted CD4+ T cells), and recognizes an antigen fragment (such as a gluten peptide) presented on MHC molecules including HLA-DQ2.5.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions

In one aspect, the invention is based, in part, on the binding of an anti-HLA-DQ2.5 antibody to HLA-DQ2.5 that presents a gluten peptide to T cells. In certain embodiments, antibodies that bind to HLA-DQ2.5 are provided.

A. Exemplary Anti-HLA-DQ2.5 Antibodies

In one aspect, the invention provides isolated antibodies that has binding activity to HLA-DQ2.5. In certain embodiments, the anti-HLA-DQ2.5 antibody (“the antibody”) has the functions/characteristics below.

The antibody has binding activity to HLA-DQ2.5. In other words, the antibody binds to HLA-DQ2.5. More preferably, the antibody has specific binding activity to HLA-DQ2.5. That is, the antibody specifically binds to HLA-DQ2.5. Due to the similarity among HLA-DQ2.5, HLA-DQ2.2, and HLA-DQ7.5, the anti-HLA-DQ2.5 antibody may also specifically binds to (or have specific binding activity to) HLA-DQ2.2 and/or HLA-DQ7.5.

The antibody has substantially no binding activity to HLA-DR/DP, i.e., the antibody does not substantially bind to HLA-DR/DP. In other words, the antibody has no specific binding activity to HLA-DR/DP or no significant binding activity to HLA-DR/DP. That is, the antibody does not specifically bind to HLA-DR/DP or significantly bind to HLA-DR/DP. Similarly, the antibody has substantially no binding activity to an HLA-DQ molecule other than HLA-DQ2.5, HLA-DQ2.2, or HLA-DQ7.5, such as HLA-DQ8, HLA-DQ5.1, HLA-DQ6.3, and HLA-DQ7.3, i.e., the antibody does not substantially bind to an HLA-DQ molecule other than HLA-DQ2.5, HLA-DQ2.2, or HLA-DQ7.5, such as HLA-DQ8, HLA-DQ5.1, HLA-DQ6.3, and HLA-DQ7.3. In other words, the antibody has no specific/significant binding activity to an HLA-DQ molecule other than HLA-DQ2.5, HLA-DQ2.2, or HLA-DQ7.5, such as HLA-DQ8, HLA-DQ5.1, HLA-DQ6.3, and HLA-DQ7.3. That is, the antibody does not specifically/significantly bind to an HLA-DQ molecule other than HLA-DQ2.5, HLA-DQ2.2, or HLA-DQ7.5, such as HLA-DQ8, HLA-DQ5.1, HLA-DQ6.3, and HLA-DQ7.3. To prevent any substantial inhibitory effects on these non-target MHC class II molecules, and to improve antibody PK for the celiac disease patients who are HLA-DQ2.5 heterozygous patients, these characteristics are preferable.

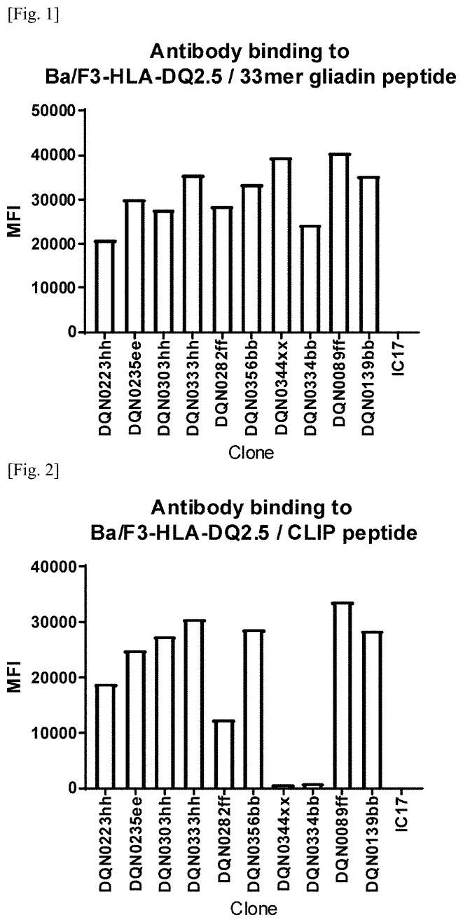

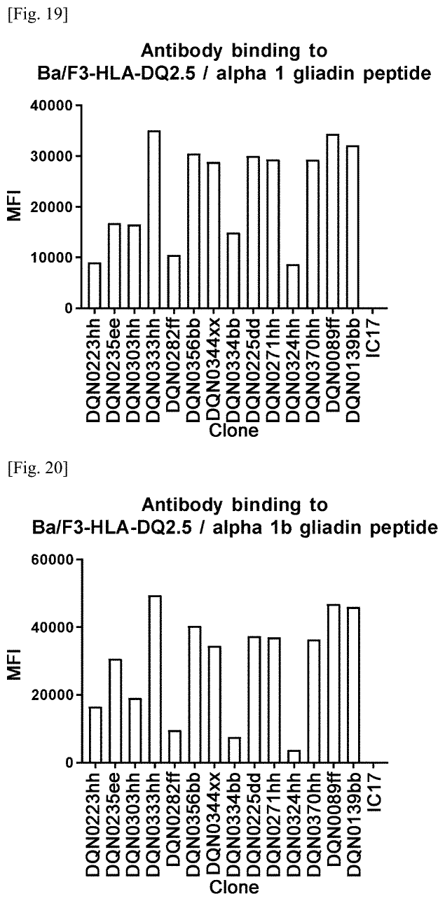

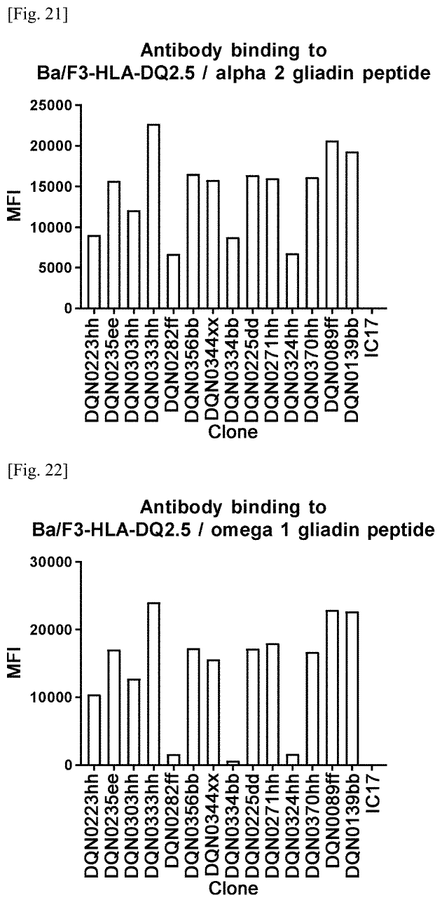

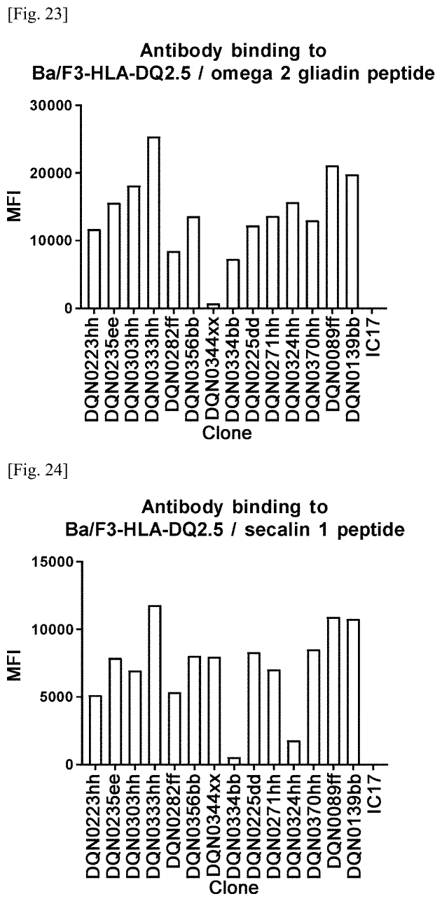

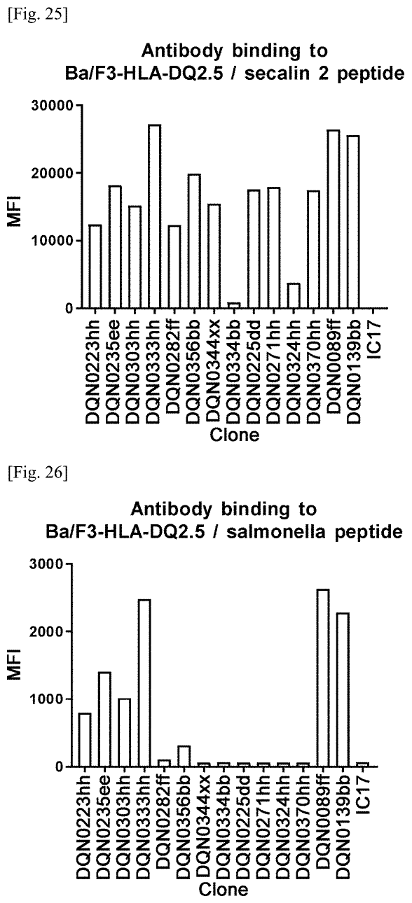

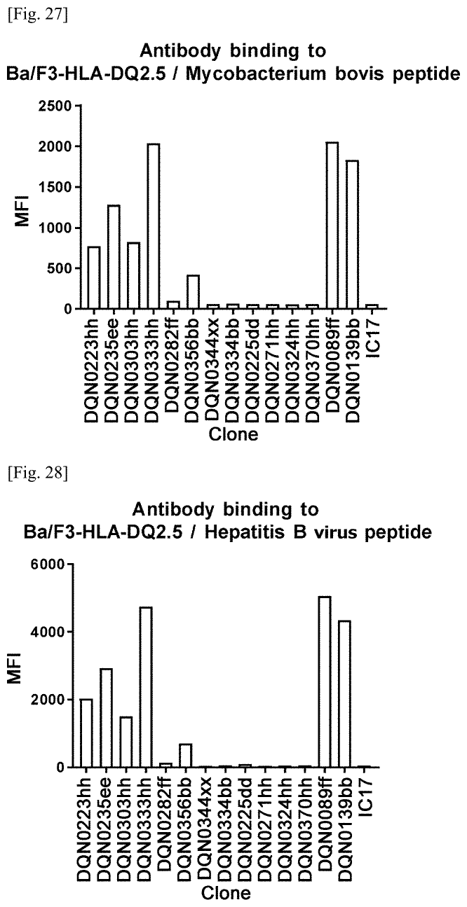

*The feature of the “substantially no binding activity” can be defined, for example, as described in the FACS results of FIGS. 2 to 11 and 16 to 38. The antibody having “substantially no binding activity” to a specific antigen has an MFI (Mean Fluorescence Intensity) value that is 300% or less, preferably 200% or less, more preferably 150% or less of the MFI value of the negative control under the measurement conditions of Example 4.1, 4.2 and 7 to 10.

In another aspect, the antibody having “substantially no binding activity” to a specific antigen has an MFI value that is 5% or less, preferably 4% or less, more preferably 3% or less when taking a MFI value of the IC17 as 0% and a MFI value of the DQN00139bb as 100% under the measurement conditions of Example 4.1 and 4.2.

The antibody has binding activity to HLA-DQ2.5 that is in complex with a gluten peptide. Herein, a complex formed between an HLA-DQ2.5 molecule and a gluten peptide is referred to as “an HLA-DQ2.5/gluten peptide complex” or “HLA-DQ2.5/gluten peptide”. It may also be rephrased as, for example, “HLA-DQ2.5 loaded with a gluten peptide”, “gluten peptide-loaded HLA-DQ2.5”, “HLA-DQ2.5 bound by a gluten peptide”, “HLA-DQ2.5 in the form of a complex with a gluten peptide”, and “a complex of HLA-DQ2.5 and a gluten peptide”. The same applies to “an HLA-DQ2.5/gliadin peptide complex”, “an HLA-DQ2.5/33mer gliadin peptide complex”, “an HLA-DQ2.5/invariant chain complex”, “an HLA-DQ2.5/CLIP peptide complex”, “an HLA-DQ2.5/alpha 1 gliadin peptide complex”, “an HLA-DQ2.5/alpha 1b gliadin peptide complex”, “an HLA-DQ2.5/alpha 2 gliadin peptide complex”, “an HLA-DQ2.5/omega 1 gliadin peptide complex”, “an HLA-DQ2.5/omega 2 gliadin peptide complex”, “an HLA-DQ2.5/secalin 1 peptide complex”, “an HLA-DQ2.5/secalin 2 peptide complex”, “an HLA-DQ2.5/salmonella peptide complex”, “an HLA-DQ2.5/Mycobacterium bovis peptide complex” and “an HLA-DQ2.5/Hepatitis B virus peptide complex” mentioned below.

The gluten peptide is preferably a gliadin peptide. The gliadin peptide is preferably a 33mer gliadin peptide, an alpha 1 gliadin peptide, an alpha 1b gliadin peptide, an alpha 2 gliadin peptide, an omega 1 gliadin peptide or an omega 2 gliadin peptide. More preferably, the gliadin peptide is a 33mer gliadin peptide, an alpha 1 gliadin peptide, an alpha 2 gliadin peptide, an omega 1 gliadin peptide or an omega 2 gliadin peptide.

In one aspect, the gluten peptide is preferably a secalin peptide. The secalin peptide is preferably a secalin 1 peptide or a secalin 2 peptide. To prevent any substantial inhibitory effects on these non-target WIC class II molecules and HLA-DQ2.5 in the form of a complex with irrelevant peptide, and to improve antibody PK for the celiac disease patients, these characteristics are preferable.

*Anti-HLA-DQ2.5 antibodies of the invention have a dissociation constant (Kd) of 5×10⁻⁷ M or less, preferably 5×10⁻⁸M or less, more preferably 1×10⁻⁸M or less, still more preferably 7×10⁻⁹M or less for binding to at least one, two, three, four, five, six, seven or all of the group consisting of an HLA-DQ2.5/33mer gliadin peptide complex, an HLA-DQ2.5/alpha 1 gliadin peptide complex, an HLA-DQ2.5/alpha 1b gliadin peptide complex, an HLA-DQ2.5/alpha 2 gliadin peptide complex, an HLA-DQ2.5/omega 1 gliadin peptide complex, an HLA-DQ2.5/omega 2 gliadin peptide complex, an HLA-DQ2.5/secalin 1 peptide complex and an HLA-DQ2.5/secalin 2 peptide complex.

In another aspect, Anti-HLA-DQ2.5 antibodies of the invention have a dissociation constant (Kd) of 5×10⁻⁷M or less, preferably 5×10⁻⁸M or less, more preferably 1×10⁻⁸ M or less, still more preferably 7×10⁻⁹M or less for binding to preferably an HLA-DQ2.5/33mer gliadin peptide complex.

The antibody has neutralizing activity against the binding between HLA-DQ2.5 and TCR. In other words, the antibody blocks the binding between HLA-DQ2.5 and TCR. More preferably, such binding occurs in the presence of a gluten peptide, i.e., when HLA-DQ2.5 is bound by a gluten peptide, or forms a complex with a gluten peptide. The gluten peptide is preferably a gliadin peptide, more preferably a 33mer gliadin peptide, an alpha 1 gliadin peptide, an alpha 1b gliadin peptide, an alpha 2 gliadin peptide, an omega 1 gliadin peptide or an omega 2 gliadin peptide, still more preferably a 33mer gliadin peptide, an alpha 1 gliadin peptide, an alpha 2 gliadin peptide, an omega 1 gliadin peptide or an omega 2 gliadin peptide. In one aspect, the gluten peptide is preferably a secalin peptide, more preferably a secalin 1 peptide or a secalin 2 peptide. The antibody blocks the interaction between an HLA-DQ2.5/gluten peptide complex and an HLA-DQ2.5/gluten peptide-restricted CD4+ T cell. Preferably, the antibody blocks the interaction between an HLA-DQ2.5/gliadin peptide complex and an HLA-DQ2.5/gliadin peptide-restricted CD4+ T cell and/or the interaction between an HLA-DQ2.5/secalin peptide complex and an HLA-DQ2.5/secalin peptide-restricted CD4+ T cell, more preferably, the interaction between an HLA-DQ2.5/gliadin peptide complex and an HLA-DQ2.5/gliadin peptide-restricted CD4+ T cell.

More preferably, the antibody blocks at least one, two, three, four, five, six, seven or all of the group consisting of the interaction between an HLA-DQ2.5/33mer gliadin peptide complex and an HLA-DQ2.5/33mer gliadin peptide-restricted CD4+ T cell, the interaction between an HLA-DQ2.5/alpha 1 gliadin peptide complex and an HLA-DQ2.5/alpha 1 gliadin peptide-restricted CD4+ T cell, the interaction between an HLA-DQ2.5/alpha 1b gliadin peptide complex and an HLA-DQ2.5/alpha 1b gliadin peptide-restricted CD4+ T cell, the interaction between an HLA-DQ2.5/alpha 2 gliadin peptide complex and an HLA-DQ2.5/alpha 2 gliadin peptide-restricted CD4+ T cell, the interaction between an HLA-DQ2.5/omega 1 gliadin peptide complex and an HLA-DQ2.5/omega 1 gliadin peptide-restricted CD4+ T cell, the interaction between an HLA-DQ2.5/omega 2 gliadin peptide complex and an HLA-DQ2.5/omega 2 gliadin peptide-restricted CD4+ T cell, the interaction between an HLA-DQ2.5/secalin 1 peptide complex and an HLA-DQ2.5/secalin 1 peptide-restricted CD4+ T cell and the interaction between an HLA-DQ2.5/secalin 2 peptide complex and an HLA-DQ2.5/secalin 2 peptide-restricted CD4+ T cell.

Still more preferably, the antibody blocks at least one, two, three, four or all of the group consisting of the interaction between an HLA-DQ2.5/33mer gliadin peptide complex and an HLA-DQ2.5/33mer gliadin peptide-restricted CD4+ T cell, the interaction between an HLA-DQ2.5/alpha 1 gliadin peptide complex and an HLA-DQ2.5/alpha 1 gliadin peptide-restricted CD4+ T cell, the interaction between an HLA-DQ2.5/alpha 2 gliadin peptide complex and an HLA-DQ2.5/alpha 2 gliadin peptide-restricted CD4+ T cell, the interaction between an HLA-DQ2.5/omega 1 gliadin peptide complex and an HLA-DQ2.5/omega 1 gliadin peptide-restricted CD4+ T cell and the interaction between an HLA-DQ2.5/omega 2 gliadin peptide complex and an HLA-DQ2.5/omega 2 gliadin peptide-restricted CD4+ T cell.

Still more preferably, the antibody blocks the interaction between an HLA-DQ2.5/33mer gliadin peptide complex and an HLA-DQ2.5/33mer gliadin peptide-restricted CD4+ T cell, the interaction between an HLA-DQ2.5/alpha 1 gliadin peptide complex and an HLA-DQ2.5/alpha 1 gliadin peptide-restricted CD4+ T cell, the interaction between an HLA-DQ2.5/alpha 2 gliadin peptide complex and an HLA-DQ2.5/alpha 2 gliadin peptide-restricted CD4+ T cell, the interaction between an HLA-DQ2.5/omega 1 gliadin peptide complex and an HLA-DQ2.5/omega 1 gliadin peptide-restricted CD4+ T cell and the interaction between an HLA-DQ2.5/omega 2 gliadin peptide complex and an HLA-DQ2.5/omega 2 gliadin peptide-restricted CD4+ T cell.

The blocking of the interaction can be achieved by blocking of the above-mentioned binding between HLA-DQ2.5 and TCR.

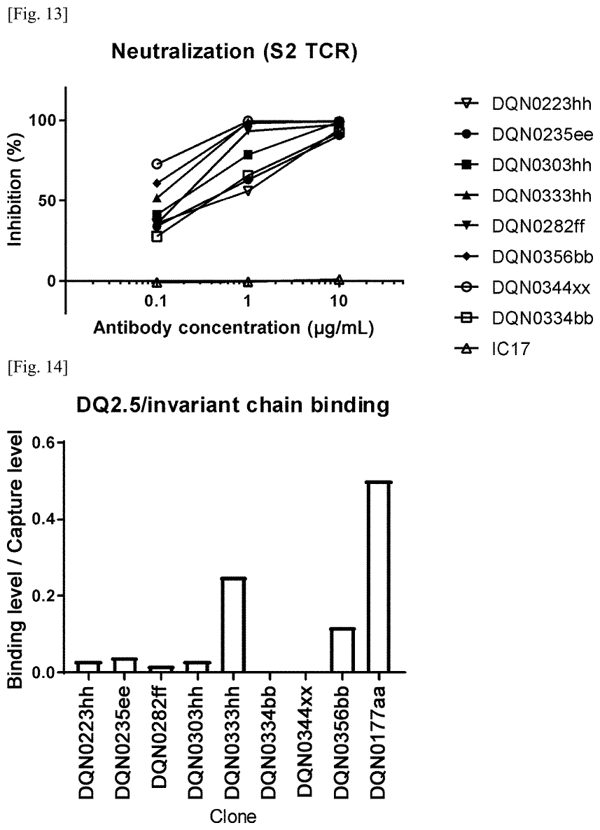

*The feature of the “neutralizing activity” can be defined, for example, as described in FIGS. 12 and 13. The antibody having the “neutralizing activity” can neutralize the binding between HLA-DQ2.5 and TCR for 95% or more, preferably 97% or more, more preferably 99% or more by antibody concentration of 1 micro g/mL under the measurement conditions described in Example 4.4.

The antibody has substantially no binding activity to (does not substantially bind to) the invariant chain (CD74). In other words, the antibody has no specific/significant binding activity to (does not specifically/significantly bind to) the invariant chain. The HLA-DQ molecules are localized on the cell surface with or without the invariant chain. When HLA-DQ forms a complex with the invariant chain, the complex on the cell surface is rapidly internalized into the endosome (rapid cell internalization called “rapid internalization”). In the endosome, the invariant chain is degraded by protease, and free HLA-DQ is loaded with a peptide such as a gluten peptide. The HLA-DQ/peptide complex is transferred to the cell surface, and then recognized by TCR on T cells. This can cause celiac disease. The complex without the invariant chain is slowly internalized into the endosome (slow cell internalization called “slow internalization”). The absence of binding to the invariant chain is preferable since the antibody is less susceptible to rapid internalization which can cause the antibody to be quickly transferred to the endosome with the invariant chain and degraded.

The antibody has substantially no binding activity to (does not substantially bind to) HLA-DQ2.5/an invariant chain. In other words, the antibody has no specific/significant binding activity to (does not specifically/significantly bond to) HLA-DQ2.5/an invariant chain. That is, the antibody does not undergo antibody internalization (“rapid internalization”) mediated by the invariant chain. These characteristics can be achieved by the above-mentioned absence of binding to the invariant chain.

The feature of the “substantially no binding activity” can be defined, for example, as described in FIG. 14. An anti-HLA-DQ2.5 antibody having “substantially no binding activity” to a specific antigen (i.e., HLA-DQ2.5-invariant chain) has a binding/capture value of 0.4 or less, i.e., level of binding of an anti-HLA-DQ2.5 antibody to HLA-DQ2.5-invariant chain/level of the anti-HLA-DQ2.5 antibody being captured, under the measurement conditions described in Example 4.5.

Additionally, some of the antibodies of the invention have binding activity to HLA-DQ2.2 and substantially no binding activity to HLA-DQ7.5. Based on the alignment information of Table 6 below, it is expected that these antibodies have binding activity to the beta chain of HLA-DQ2.5.

Other antibodies of the invention have binding activity to HLA-DQ7.5 and substantially no binding activity to HLA-DQ2.2. Based on the alignment information, it is expected that these antibodies have binding activity to the alpha chain of HLA-DQ2.5.

Other antibodies of the invention have substantially no binding activity to HLA-DQ2.2 or HLA-DQ7.5. Preferably, these antibodies have enhanced binding activity to HLA-DQ2.5 in the form of a complex with a gluten peptide. In other words, these antibodies have stronger binding activity to HLA-DQ2.5 in the form of a complex with a gluten peptide than to HLA-DQ2.5 in the form of a complex with a peptide other than a gluten peptide, or than to HLA-DQ2.5 which is not in the form of a complex with any peptide.

More preferably, these antibodies have stronger binding activity to at least one, two, three, four, five, six, seven or all of the group consisting of HLA-DQ2.5/33mer gliadin peptide, HLA-DQ2.5/alpha 1 gliadin peptide, HLA-DQ2.5/alpha 1b gliadin peptide, HLA-DQ2.5/alpha 2 gliadin peptide, HLA-DQ2.5/omega 1 gliadin peptide, HLA-DQ2.5/omega 2 gliadin peptide, HLA-DQ2.5/secalin 1 peptide and HLA-DQ2.5/secalin 2 peptide than to at least one, two, three, four or all of the group consisting of HLA-DQ2.5/CLIP peptide, HLA-DQ2.5/salmonella peptide, HLA-DQ2.5/Mycobacterium bovis peptide, HLA-DQ2.5/Hepatitis B virus peptide and HLA-DQ2.5 positive PBMC-B cell.

Still more preferably, these antibodies have stronger binding activity to at least one, two, three, four or all of the group consisting of HLA-DQ2.5/33mer gliadin peptide, HLA-DQ2.5/alpha 1 gliadin peptide, HLA-DQ2.5/alpha 2 gliadin peptide, HLA-DQ2.5/omega 1 gliadin peptide, HLA-DQ2.5/omega 2 gliadin peptide than to at least one, two, three, four of the group consisting of HLA-DQ2.5/CLIP peptide, HLA-DQ2.5/salmonella peptide, HLA-DQ2.5/Mycobacterium bovis peptide, HLA-DQ2.5/Hepatitis B virus peptide and HLA-DQ2.5 positive PBMC-B cell.

Still more preferably, these antibodies have stronger binding activity to HLA-DQ2.5/33mer gliadin peptide, HLA-DQ2.5/alpha 1 gliadin peptide, HLA-DQ2.5/alpha 2 gliadin peptide, HLA-DQ2.5/omega 1 gliadin peptide and HLA-DQ2.5/omega 2 gliadin peptide than to HLA-DQ2.5/CLIP peptide, HLA-DQ2.5/salmonella peptide, HLA-DQ2.5/Mycobacterium bovis peptide, HLA-DQ2.5/Hepatitis B virus peptide and HLA-DQ2.5 positive PBMC-B cell.

Additionally, these antibodies have binding activity to HLA-DQ2.5 in the form of a complex with a gluten peptide and substantially no binding activity to HLA-DQ2.5 in the form of a complex with a peptide other than a gluten peptide, or substantially no binding activity to HLA-DQ2.5 which is not in the form of a complex with any peptide.

More preferably, these antibodies have binding activity to at least one, two, three, four, five, six, seven or all of the group consisting of HLA-DQ2.5/33mer gliadin peptide, HLA-DQ2.5/alpha 1 gliadin peptide, HLA-DQ2.5/alpha 1b gliadin peptide, HLA-DQ2.5/alpha 2 gliadin peptide, HLA-DQ2.5/omega 1 gliadin peptide, HLA-DQ2.5/omega 2 gliadin peptide, HLA-DQ2.5/secalin 1 peptide and HLA-DQ2.5/secalin 2 peptide and substantially no binding activity to at least one, two, three, four or all of the group consisting of HLA-DQ2.5/CLIP peptide, HLA-DQ2.5/salmonella peptide, HLA-DQ2.5/Mycobacterium bovis peptide, HLA-DQ2.5/Hepatitis B virus peptide and HLA-DQ2.5 positive PBMC-B cell.

Still more preferably, these antibodies have binding activity to at least one, two, three, four or all of the group consisting of HLA-DQ2.5/33mer gliadin peptide, HLA-DQ2.5/alpha 1 gliadin peptide, HLA-DQ2.5/alpha 2 gliadin peptide, HLA-DQ2.5/omega 1 gliadin peptide, HLA-DQ2.5/omega 2 gliadin peptide and substantially no binding activity to at least one, two, three, four or all of the group consisting of HLA-DQ2.5/CLIP peptide, HLA-DQ2.5/salmonella peptide, HLA-DQ2.5/Mycobacterium bovis peptide, HLA-DQ2.5/Hepatitis B virus peptide and HLA-DQ2.5 positive PBMC-B cell.

Still more preferably, these antibodies have binding activity to HLA-DQ2.5/33mer gliadin peptide, HLA-DQ2.5/alpha 1 gliadin peptide, HLA-DQ2.5/alpha 2 gliadin peptide, HLA-DQ2.5/omega 1 gliadin peptide and HLA-DQ2.5/omega 2 gliadin peptide and substantially no binding activity to HLA-DQ2.5/CLIP peptide, HLA-DQ2.5/salmonella peptide, HLA-DQ2.5/Mycobacterium bovis peptide, HLA-DQ2.5/Hepatitis B virus peptide and HLA-DQ2.5 positive PBMC-B cell.

In one aspect, the invention provides an anti-HLA-DQ2.5 antibody comprising at least one, two, three, four, five, or six HVRs (CDRs) selected from (a) HVR-H1 (HCDR1) comprising the amino acid sequence of any one of SEQ ID NOs: 13 to 23 and 146 to 149; (b) HVR-H2 (HCDR2) comprising the amino acid sequence of any one of SEQ ID NOs: 25 to 35 and 150 to 153; (c) HVR-H3 (HCDR3) comprising the amino acid sequence of any one of SEQ ID NOs: 37 to 47 and 154 to 157; (d) HVR-L1 (LCDR1) comprising the amino acid sequence of any one of SEQ ID NOs: 61 to 71 and 162 to 165; (e) HVR-L2 (LCDR2) comprising the amino acid sequence of any one of SEQ ID NOs: 73 to 83 and 166 to 169; and (f) HVR-L3 (LCDR3) comprising the amino acid sequence of any one of SEQ ID NOs: 85 to 95 and 170 to 173.

In one aspect, the invention provides an antibody comprising at least one or two, or all three of the VH HVR (HCDR) sequences selected from (a) HVR-H1 (HCDR1) comprising the amino acid sequence of any one of SEQ ID NOs: 13 to 23 and 146 to 149; (b) HVR-H2 (HCDR2) comprising the amino acid sequence of any one of SEQ ID NOs: 25 to 35 and 150 to 153; and (c) HVR-H3 (HCDR3) comprising the amino acid sequence of any one of SEQ ID NOs: 37 to 47 and 154 to 157.

In another aspect, the invention provides an antibody comprising at least one or two, or all three of the VL HVR (LCDR) sequences selected from (a) HVR-L1 (LCDR1) comprising the amino acid sequence of any one of SEQ ID NOs: 61 to 71 and 162 to 165; (b) HVR-L2 (LCDR2) comprising the amino acid sequence of any one of SEQ ID NOs: 73 to 83 and 166 to 169; and (c) HVR-L3 (LCDR3) comprising the amino acid sequence of any one of SEQ ID NOs: 85 to 95 and 170 to 173.

In another aspect, an antibody of the invention comprises (a) a VH domain comprising at least one or two, or all three of the VH HVR (HCDR) sequences selected from (i) HVR-H1 (HCDR1) comprising the amino acid sequence of any one of SEQ ID NOs: 13 to 23 and 146 to 149, (ii) HVR-H2 (HCDR2) comprising the amino acid sequence of any one of SEQ ID NOs: 25 to 35 and 150 to 153, and (iii) HVR-H3 (HCDR3) comprising an amino acid sequence of any one of SEQ ID NOs: 37 to 47 and 154 to 157; and (b) a VL domain comprising at least one or two, or all three of the VL HVR (LCDR) sequences selected from (i) HVR-L1 (LCDR1) comprising the amino acid sequence of any one of SEQ ID NOs: 61 to 71 and 162 to 165, (ii) HVR-L2 (LCDR2) comprising the amino acid sequence of any one of SEQ ID NOs: 73 to 83 and 166 to 169, and (c) HVR-L3 (LCDR3) comprising the amino acid sequence of any one of SEQ ID NOs: 85 to 95 and 170 to 173.

In another aspect, the invention provides an antibody comprising (a) HVR-H1 (HCDR1) comprising the amino acid sequence of any one of SEQ ID NOs: 13 to 23 and 146 to 149; (b) HVR-H2 (HCDR2) comprising the amino acid sequence of any one of SEQ ID NOs: 25 to 35 and 150 to 153; (c) HVR-H3 (HCDR3) comprising the amino acid sequence of any one of SEQ ID NOs: 37 to 47 and 154 to 157; (d) HVR-L1 (LCDR1) comprising the amino acid sequence of any one of SEQ ID NOs: 61 to 71 and 162 to 165; (e) HVR-L2 (LCDR2) comprising the amino acid sequence of any one of SEQ ID NOs: 73 to 83 and 166 to 169; and (f) HVR-L3 (LCDR3) comprising an amino acid sequence selected from any one of SEQ ID NOs: 85 to 95 and 170 to 173.

In another aspect, the sequence ID numbers (SEQ ID NOs) of the VH, VL, HCDR1, HCDR2, HCDR3, LCDR1, LCDR2, and LCDR3 sequences for the antibodies of the present invention are as follows:

TABLE 1

| Antibody | SEQ ID NO: | | | | | | | |
|---|---|---|---|---|---|---|---|---|
| Name | VH | HCDR1 | HCDR2 | HCDR3 | VL | LCDR1 | LCDR2 | LCDR3 |
| DQN0223hh | 1 | 13 | 25 | 37 | 49 | 61 | 73 | 85 |
| DQN0235ee | 2 | 14 | 26 | 38 | 50 | 62 | 74 | 86 |
| DQN0303hh | 3 | 15 | 27 | 39 | 51 | 63 | 75 | 87 |
| DQN0333hh | 4 | 16 | 28 | 40 | 52 | 64 | 76 | 88 |
| DQN0282ff | 5 | 17 | 29 | 41 | 53 | 65 | 77 | 89 |
| DQN0356bb | 6 | 18 | 30 | 42 | 54 | 66 | 78 | 90 |
| DQN0344xx | 7 | 19 | 31 | 43 | 55 | 67 | 79 | 91 |
| DQN0334bb | 8 | 20 | 32 | 44 | 56 | 68 | 80 | 92 |
| DQN0139bb | 9 | 21 | 33 | 45 | 57 | 69 | 81 | 93 |
| DQN0225dd | 142 | 146 | 150 | 154 | 158 | 162 | 166 | 170 |
| DQN0271hh | 143 | 147 | 151 | 155 | 159 | 163 | 167 | 171 |
| DQN0324hh | 144 | 148 | 152 | 156 | 160 | 164 | 168 | 172 |
| DQN0370hh | 145 | 149 | 153 | 157 | 161 | 165 | 169 | 173 |
| DQN0177aa | 10 | 22 | 34 | 46 | 58 | 70 | 82 | 94 |
| DQN0089ff | 11 | 23 | 35 | 47 | 59 | 71 | 83 | 95 |

In certain embodiments, any one or more amino acids of an anti-HLA-DQ2.5 antibody as provided above are substituted in any of the HVR positions.

In certain embodiments, the substitutions are conservative substitutions, as provided herein.

In any of the above embodiments, an anti-HLA-DQ2.5 antibody is humanized. In one embodiment, an anti-HLA-DQ2.5 antibody comprises HVRs as in any of the above embodiments, and further comprises an acceptor human framework, e.g., a human immunoglobulin framework or a human consensus framework. In another embodiment, an anti-HLA-DQ2.5 antibody comprises HVRs as in any of the above embodiments, and further comprises the FR1, FR2, FR3, or FR4 sequence shown in Tables 2 and 3 below.

In another aspect, an anti-HLA-DQ2.5 antibody comprises a heavy-chain variable domain (VH) sequence having at least 90%, 91%, 92%, 93%, 94%, 95%, 96%, 97%, 98%, 99%, or 100% sequence identity to the amino acid sequence of any one of SEQ ID NOs: 1 to 11 and 142 to 145. In certain embodiments, a VH sequence having at least 90%, 91%, 92%, 93%, 94%, 95%, 96%, 97%, 98%, or 99% identity comprises substitutions (e.g., conservative substitutions), insertions, or deletions relative to the reference sequence, but an anti-HLA-DQ2.5 antibody comprising that sequence retains the ability to bind to HLA-DQ2.5. In certain embodiments, a total of 1 to 10 amino acids have been substituted, inserted and/or deleted in any one of SEQ ID NOs: 1 to 11 and 142 to 145. In certain embodiments, substitutions, insertions, or deletions occur in regions outside the HVRs (i.e., in the FRs). Optionally, the anti-HLA-DQ2.5 antibody comprises the VH sequence of any one of SEQ ID NOs: 1 to 11 and 142 to 145 or a sequence comprising a post-translational modification thereof. In a particular embodiment, the VH comprises one, two or three HVRs selected from: (a) HVR-H1 comprising the amino acid sequence of any one of SEQ ID NOs: 13 to 23 and 146 to 149, (b) HVR-H2 comprising the amino acid sequence of any one of SEQ ID NOs: 25 to 35 and 150 to 153, and (c) HVR-H3 comprising the amino acid sequence of any one of SEQ ID NOs: 37 to 47 and 154 to 157. Post-translational modifications include but are not limited to a modification of glutamine or glutamate at the N terminus of the heavy chain or light chain to pyroglutamic acid by pyroglutamylation.

In another aspect, an anti-HLA-DQ2.5 antibody is provided, wherein the antibody comprises a light-chain variable domain (VL) having at least 90%, 91%, 92%, 93%, 94%, 95%, 96%, 97%, 98%, 99%, or 100% sequence identity to the amino acid sequence of any one of SEQ ID NOs: 49 to 59 and 158 to 161. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-HLA-DQ2.5 antibody comprising that sequence retains the ability to bind to HLA-DQ2.5. In certain embodiments, a total of 1 to 10 amino acids have been substituted, inserted and/or deleted in any one of SEQ ID NOs: 49 to 59 and 158 to 161. In certain embodiments, the substitutions, insertions, or deletions occur in regions outside the HVRs (i.e., in the FRs). Optionally, the anti-HLA-DQ2.5 antibody comprises the VL sequence of any one of SEQ ID NOs: 49 to 59 and 158 to 161 or a sequence comprising a post-translational modification thereof. In a particular embodiment, the VL comprises one, two or three HVRs selected from (a) HVR-L1 comprising the amino acid sequence of any one of SEQ ID NOs: 61 to 71 and 162 to 165; (b) HVR-L2 comprising the amino acid sequence of any one of SEQ ID NOs: 73 to 83 and 166 to 169; and (c) HVR-L3 comprising the amino acid sequence of any one of SEQ ID NOs: 85 to 95 and 170 to 173. Post-translational modifications include but are not limited to a modification of glutamine or glutamate at the N terminus of the heavy chain or light chain to pyroglutamic acid by pyroglutamylation.

In another aspect, an anti-HLA-DQ2.5 antibody is provided, wherein the antibody comprises a VH as in any of the embodiments provided above, and a VL as in any of the embodiments provided above. In one embodiment, the antibody comprises the VH sequence of any one of SEQ ID NOs: 1 to 10 and 142 to 145 or a sequence comprising a post-translational modification thereof, and the VL sequence of any one of SEQ ID NOs: 49 to 59 and 158 to 161 or a sequence comprising a post-translational modification thereof. Post-translational modifications include but are not limited to a modification of glutamine or glutamate at the N terminus of the heavy chain or light chain to pyroglutamic acid by pyroglutamylation.

In a further aspect, the invention provides an antibody that binds to the same epitope as an anti-HLA-DQ2.5 antibody provided herein. For example, in certain embodiments, an antibody is provided that binds to the same epitope as any of the above-mentioned antibodies. In certain embodiments, an antibody is provided that binds to an epitope within a fragment of HLA-DQ2.5 consisting of about 8 to 17 amino acids.

In a further aspect of the invention, an anti-HLA-DQ2.5 antibody according to any of the above embodiments is a monoclonal antibody, including a chimeric, humanized or human antibody. In one embodiment, an anti-HLA-DQ2.5 antibody is an antibody fragment, e.g., a Fv, Fab, Fab′, scFv, diabody, or F(ab′)₂ fragment. In another embodiment, the antibody is a full-length antibody, e.g., an intact IgG₁ antibody or other antibody class or isotype as defined herein.

In a further aspect, an anti-HLA-DQ2.5 antibody according to any of the above embodiments may incorporate any of the features described in Sections 1-7 below, whether singly or in combination:

1. Antibody Affinity

In certain embodiments, an antibody provided herein has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (MA). In one embodiment, an MA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20 ‘; Packard) is added, and the plates are counted on a TOPCOUNT’ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE (registered trademark)-3000 (BIAcore, Inc., Piscataway, N.J.) is performed at 25 degrees C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NETS) according to the supplier's instructions. Antigen is diluted with 10 mM sodium acetate, pH 4.8, to 5 micro g/ml (˜0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE′ technology; U.S. Pat. No. 5,770,429 describing HUMAB (registered trademark) technology; U.S. Pat. No. 7,041,870 describing K-M MOUSE (registered trademark) technology, and U.S. Patent Application Publication No. US 2007/0061900, describing VELOCIMOUSE (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

a) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

b) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g. a substitution) at one or more amino acid positions.

Antibodies with increased half lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

c) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

d) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In one embodiment, isolated nucleic acid encoding an anti-HLA-DQ2.5 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an anti-HLA-DQ2.5 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-HLA-DQ2.5 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR″ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

C. Assays

Anti-HLA-DQ2.5 antibodies provided herein may be identified, screened for, or characterized for their physical/chemical properties and/or biological activities by various assays known in the art.

1. Binding Assays and Other Assays

In one aspect, an antibody of the invention is tested for its antigen binding activity, e.g., by known methods such as ELISA, Western blot, etc.

In another aspect, competition assays may be used to identify an antibody that competes with, for example, any of the above-mentioned antibodies for binding to HLA-DQ2.5. In certain embodiments, such a competing antibody binds to the same epitope (e.g., a linear or a conformational epitope) that is bound by the above-mentioned antibodies. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized HLA-DQ2.5 is incubated in a solution comprising a first labeled antibody that binds to HLA-DQ2.5 and a second unlabeled antibody that is being tested for its ability to compete with the first antibody for binding to HLA-DQ2.5. The second antibody may be present in a hybridoma supernatant. As a control, immobilized HLA-DQ2.5 is incubated in a solution comprising the first labeled antibody but not the second unlabeled antibody. After incubation under conditions permissive for binding of the first antibody to HLA-DQ2.5, excess unbound antibody is removed, and the amount of label associated with immobilized HLA-DQ2.5 is measured. If the amount of label associated with immobilized HLA-DQ2.5 is substantially reduced in the test sample relative to the control sample, then that indicates that the second antibody is competing with the first antibody for binding to HLA-DQ2.5. See Harlow and Lane (1988) Antibodies: A Laboratory Manual ch. 14 (Cold Spring Harbor Laboratory, Cold Spring Harbor, N.Y.).

2. Activity Assays/Screening Method

In one aspect, assays are provided for identifying anti-HLA-DQ2.5 antibodies having a binding/biological activity. Such assays can be used in screening methods of the present invention.

In some embodiments, the present invention provides a method of screening for an anti-HLA-DQ2.5 antibody, which comprises: (a) testing whether an antibody has binding activity to HLA-DQ2.5; and selecting an antibody that has binding activity to HLA-DQ2.5; (b) testing whether an antibody has a specific binding activity to HLA-DR or DP; and selecting an antibody that has no specific binding activity to HLA-DR or DP; (c) testing whether an antibody has a specific binding activity to a complex of the invariant chain and HLA-DQ2.5; and selecting an antibody that has no specific binding activity to the complex of the invariant chain and HLA-DQ2.5. The antibody selected in step (a) above may also have binding activity to HLA-DQ2.2 and/or HLA-DQ7.5 due to their similarity to HLA-DQ2.5. This binding activity may be specific binding activity.

In certain embodiments, the method of the present invention further comprises: testing whether an antibody binds to (or has binding activity to) HLA-DQ2.5 in the presence of a gluten peptide; and selecting an antibody that binds to (or has binding activity to) HLA-DQ2.5 in the presence of a gluten peptide. Preferably, the gliten peptide is gliadin.

In certain embodiments, the method of the present invention further comprises: testing whether an antibody has neutralizing activity against the binding between HLA-DQ2.5 and TCR; and selecting an antibody that has the neutralizing activity.

Before performing the steps below, candidate anti-HLA-DQ2.5 antibodies may be prepared by any methods, for example, as mentioned in Example 3.

Animals such as rabbits, mice, rats, and other animals suitable for immunization are immunized with an antigen (e.g., HLA-DQ2.5 optionally bound by a gliadin peptide). The antigen may be prepared as a recombinant protein using any methods, for example, as mentioned in Examples 1 and 2. Antibody-containing samples such as the blood and spleen are collected from the immunized animals. For B cell selection, for example, a biotinylated antigen is prepared, and antigen-binding B cells are bound by the biotinylated antigen, and the cells are subjected to cell sorting and culturing for selection. Specific binding of the cells to the antigen may be evaluated by any suitable method such as the ELISA method. This method may also be used for assessing the absence of cross-reactivity towards antigens of no interest. To isolate or determine the sequence of the selected antibody, for example, RNAs are purified from the cells, and DNAs encoding regions of the antibody are prepared by reverse transcription of the RNAs and PCR amplification. Furthermore, cloned antibody genes may be expressed in suitable cells, and the antibody may be purified from the culture supernatants for further analysis.

To test whether an anti-HLA-DQ2.5 antibody binds to an antigen of interest (e.g., HLA-DQ2.5 and HLA-DQ2.5 bound by a gluten peptide such as gliadin) or an antigen of no interest (e.g., HLA-DR, HLA-DP, the invariant chain, and a complex of the invariant chain and HLA-DQ2.5), any methods for assessing the binding can be used. For example, when an FACS-based cell sorting method is used, cells expressing the antigen (e.g., HLA-DQ2.5 HLA-DR, or HLA-DP) are incubated with the tested antibody, and then a suitable secondary antibody against the tested (i.e., primary) antibody is added and incubated. The binding between the antigen and the tested antibody is detected by FACS analysis using, for example, a chromogenic/fluorescent label attached to the secondary antibody (for example, as mentioned in Example 4.1). Alternatively, any of the measurement methods mentioned in “1. Antibody Affinity” in this specification can be utilized. For example, the measurement of Kd by a BIACORE surface plasmon resonance assay can be used for assessing the binding between the tested antibody and HLA-DQ in the presence of a gluten peptide (e.g., gliadin) or the invariant chain (for example, as mentioned in Examples 4.3 and 4.5).

In certain embodiments, the method of the present invention further comprises: testing whether the antibody has neutralizing activity against the binding between HLA-DQ2.5 and TCR (or the interaction between HLA-DQ2.5 and HLA-DQ2.5-restricted CD4+ T cells); and selecting the antibody that has the neutralizing activity. These steps can be performed in the presence of a gluten peptide such as a gliadin peptide, i.e., using HLA-DQ2.5 bound by the peptide. The neutralizing activity can be assessed, for example, as mentioned in Example 4.4. Briefly, beads such as streptavidin-coated yellow particles are appropriately prepared, and soluble HLA-DQ bound by a gliadin peptide is added to the beads for immobilization on a plate. The plate is washed and blocked, and the antibody is added thereto and incubated. When the binding between HLA-DQ2.5 and TCR is assessed, for example, D2 TCR tetramer-PE may be added and incubated. Binding between the two may be evaluated based on the chromogenic/fluorescent label of TCR bound by HLA-DQ2.5.

In certain embodiments, the method of the present invention further comprises: testing whether the antibody is internalized into the cell with the invariant chain; and selecting the antibody that is not (substantially) internalized into the cell with the invariant chain. The cell internalization (“rapid internalization” mentioned above) can be assessed by FACS analysis. Briefly, a chromogenic/fluorescent label (e.g., AlexaFluor 555) is attached to the tested antibody, and this is incubated with suitable cells in the presence or absence of Cytochalasin D which blocks the delivery of class II/invariant chain complexes to lysosomes. Then, an appropriate secondary antibody against the tested antibody (i.e., primary antibody), for example, an anti-human IgG Fc antibody with FITC, is added and incubated. This is subjected to FACS measurement, and the rate of invariant chain-dependent cell internalization of the antibody is calculated from values obtained in the absence and presence of Cytochalasin D. If these values are equal or comparable to each other, it can be said that the antibody is not internalized with the invariant chain.

### EXAMPLES

The following are examples of compositions of the invention. It is understood that various other embodiments may be practiced, given the general description provided above.

### Example 1

Expression and Purification of Recombinant Proteins

1.1. Expression and purification of recombinant HLA-DQ2.5/33mer gliadin peptide complex, HLA-DQ8/gliadin peptide complex, HLA-DQ5.1/DBY peptide complex, HLA-DQ2.2/CLIP peptide complex, and HLA-DQ7.5/CLIP peptide complex

Expression and Purification of Recombinant HLA-DQ2.5/33Mer Gliadin Peptide Complex

The sequences used for expression and purification are: HLA-DQA1*0501 (Protein Data Bank accession code 4OZG) and HLA-DQB1*0201 (Protein Data Bank accession code 4OZG), both of which have a CAMPATH-1H signal sequence: MGWSCIILFLVATATGVHS (SEQ ID NO: 99). HLA-DQA1*0501 has C47S mutation, GGGG linker (SEQ ID NO: 100) and c-fos leucine zipper sequence (PNAS, 1998 Sep. 29; 95(20): 11828-33) and a Flag-tag on the C-terminus of HLA-DQA1*0501. HLA-DQB1*0201 has 33-mer gliadin peptide sequence: LQLQPFPQPELPYPQPELPYPQPELPYPQPQPF (SEQ ID NO: 101), and factor X cleavage linker (Acta Crystallogr Sect F Struct Biol Cryst Commun. 2007 Dec. 1; 63 (Pt 12): 1021-1025.) on the N-terminus of HLA-DQB1*0201, GGGGG linker (SEQ ID NO: 102) and c-jun leucine zipper sequence (PNAS, 1998 Sep. 29; 95(20): 11828-33), GGGGG linker (SEQ ID NO: 102), and BAP sequence (BMC Biotechnol. 2008; 8: 41), 8× His-tag on the C-terminus of HLA-DQB1*0201. A recombinant HLA-DQ2.5/33mer gliadin peptide complex was expressed transiently using FreeStyle293-F cell line (Thermo Fisher). Conditioned media expressing the HLA-DQ2.5/33mer gliadin peptide complex was incubated with an immobilized metal affinity chromatography (IMAC) resin, followed by elution with imidazole. Fractions containing the HLA-DQ2.5/33mer gliadin peptide complex were collected and subsequently subjected to a Superdex 200 gel filtration column (GE healthcare) equilibrated with 1×PBS. Fractions containing the HLA-DQ2.5/33mer gliadin peptide complex were then pooled and stored at −80 degrees C. The purified HLA-DQ2.5/33mer gliadin peptide complex was biotinylated using BirA (Avidity).

Expression and Purification of Recombinant HLA-DQ8/Gliadin Peptide Complex

The sequences used for expression and purification are: HLA-DQA1*0301 (Protein Data Bank accession code 4GG6) and HLA-DQB1*0302 (Protein Data Bank accession code 4GG6), both of which have a CAMPATH-1H signal sequence: MGWSCIILFLVATATGVHS (SEQ ID NO: 99). HLA-DQA1*0301 has SSADLVPRGGGG linker (SEQ ID NO: 104) and c-fos leucine zipper sequence (PNAS, 1998 Sep. 29; 95(20): 11828-33) and a Flag-tag on the C-terminus of HLA-DQA1*0301. HLA-DQB1*0302 has gliadin peptide sequence: QQYPSGEGSFQPSQENPQ (SEQ ID NO: 105), and factor X cleavage linker (Acta Crystallogr Sect F Struct Biol Cryst Commun. 2007 Dec. 1; 63 (Pt 12): 1021-1025.) on the N-terminus of HLA-DQB1*0302, SSADLVPRGGGGG linker (SEQ ID NO: 106) and c-jun leucine zipper sequence (PNAS, 1998 Sep. 29; 95(20): 11828-33), GGGGG linker (SEQ ID NO: 102), and BAP sequence (BMC Biotechnol. 2008; 8: 41), 8× His-tag on the C-terminus of HLA-DQB1*0302. A recombinant HLA-DQ8/gliadin peptide was expressed transiently using FreeStyle293-F cell line. Conditioned media expressing the HLA-DQ8/gliadin peptide complex was incubated with an IMAC resin, followed by elution with imidazole. Fractions containing the HLA-DQ8/gliadin peptide complex were collected and subsequently subjected to a Superdex 200 gel filtration column equilibrated with 1×PBS. Fractions containing the HLA-DQ8/gliadin peptide complex were then pooled and stored at −80 degrees C.

Expression and purification of recombinant HLA-DQ5.1/DBY peptide complex The sequences used for expression and purification are: HLA-DQA1*0101 (IMGT/HLA accession No. HLA00601) and HLA-DQB1*0501 (IMGT/HLA accession No. HLA00638), both of which have a CAMPATH-1H signal sequence: MGWSCIILFLVATATGVHS (SEQ ID NO: 99). HLA-DQA1*0101 has C30Y mutation. HLA-DQA1*0101 has SSADLVPRGGGG linker (SEQ ID NO: 104) and c-fos leucine zipper sequence (PNAS, 1998 Sep. 29; 95(20): 11828-33) and a Flag-tag on the C-terminus of HLA-DQA1*0101. HLA-DQB1*0501 has DBY peptide sequence: ATGSNCPPHIENFSDIDMGE (SEQ ID NO: 107), and factor X cleavage linker (Acta Crystallogr Sect F Struct Biol Cryst Commun. 2007 Dec. 1; 63 (Pt 12): 1021-1025.) on the N-terminus of HLA-DQB1*0501, SSADLVPRGGGGG linker (SEQ ID NO: 104) and c-jun leucine zipper sequence (PNAS, 1998 Sep. 29; 95(20): 11828-33), GGGGG linker (SEQ ID NO: 102), and BAP sequence (BMC Biotechnol. 2008; 8: 41), 8× His-tag on the C-terminus of HLA-DQB1*0501. A recombinant HLA-DQ5.1/DBY peptide complex was expressed transiently using FreeStyle293-F cell line. Conditioned media expressing the HLA-DQ5.1/DBY peptide complex was incubated with an IMAC resin, followed by elution with imidazole. Fractions containing the HLA-DQ5.1/DBY peptide complex were collected and subsequently subjected to a Superdex 200 gel filtration column equilibrated with 1×PBS. Fractions containing the HLA-DQ5.1/DBY peptide complex were then pooled and stored at −80 degrees C. The purified HLA-DQ5.1/DBY peptide was biotinylated using BirA.

Expression and Purification of Recombinant HLA-DQ2.2/CLIP Peptide Complex

The sequences used for expression and purification are: HLA-DQA1*0201 (IMGT/HLA accession No. HLA00607) and HLA-DQB1*0202 (IMGT/HLA accession No. HLA00623), both of which have a CAMPATH-1H signal sequence: MGWSCIILFLVATATGVHS (SEQ ID NO: 99). HLA-DQA1*0201 has SSADLVPRGGGG linker (SEQ ID NO: 104) and c-fos leucine zipper sequence (PNAS, 1998 Sep. 29; 95(20): 11828-33) and a Flag-tag on the C-terminus of HLA-DQA1*0201. HLA-DQB1*0202 has CLIP peptide sequence: KLPKPPKPVSKMRMATPLLMQALPMGALP (SEQ ID NO: 103), and factor X cleavage linker (Acta Crystallogr Sect F Struct Biol Cryst Commun. 2007 Dec. 1; 63 (Pt 12): 1021-1025.) on the N-terminus of HLA-DQB1*0202, SSADLVPRGGGGG linker (SEQ ID NO: 104) and c-jun leucine zipper sequence (PNAS, 1998 Sep. 29; 95(20): 11828-33), GGGGG linker (SEQ ID NO: 102), and BAP sequence (BMC Biotechnol. 2008; 8: 41), 8× His-tag on the C-terminus of HLA-DQB1*0202. A recombinant HLA-DQ2.2/CLIP peptide complex was expressed transiently using FreeStyle293-F cell line. Conditioned media expressing the HLA-DQ2.2/CLIP peptide complex was incubated with an IMAC resin, followed by elution with imidazole. Fractions containing the HLA-DQ2.2/CLIP peptide complex were collected and subsequently subjected to a Superdex 200 gel filtration column equilibrated with 1×PBS. Fractions containing the HLA-DQ2.2/CLIP peptide complex were then pooled and stored at −80 degrees C.

Expression and Purification of Recombinant HLA-DQ7.5/CLIP Peptide Complex

The sequences used for expression and purification are: HLA-DQA1*0505 (IMGT/HLA accession No. HLA00619) and HLA-DQB1*0301 (IMGT/HLA accession No. HLA00625), both of which have a CAMPATH-1H signal sequence: MGWSCIILFLVATATGVHS (SEQ ID NO: 99). HLA-DQA1*0505 has C66S mutation. HLA-DQA1*0505 has SSADLVPRGGGG linker (SEQ ID NO: 104) and c-fos leucine zipper sequence (PNAS, 1998 Sep. 29; 95(20): 11828-33) and a Flag-tag on the C-terminus of HLA-DQA1*0505. HLA-DQB1*0301 has CLIP peptide sequence: KLPKPPKPVSKMRMATPLLMQALPMGALP (SEQ ID NO: 103), and factor X cleavage linker (Acta Crystallogr Sect F Struct Biol Cryst Commun. 2007 Dec. 1; 63 (Pt 12): 1021-1025.) on the N-terminus of HLA-DQB1*0301, SSADLVPRGGGGG linker (SEQ ID NO: 104) and c-jun leucine zipper sequence (PNAS, 1998 Sep. 29; 95(20): 11828-33), GGGGG linker (SEQ ID NO: 102), and BAP sequence (BMC Biotechnol. 2008; 8: 41), 8× His-tag on the C-terminus of HLA-DQB1*0301. A recombinant HLA-DQ7.5/CLIP peptide complex was expressed transiently using FreeStyle293-F cell line. Conditioned media expressing the HLA-DQ7.5/CLIP peptide complex was incubated with an IMAC resin, followed by elution with imidazole. Fractions containing the HLA-DQ7.5/CLIP peptide complex were collected and subsequently subjected to a Superdex 200 gel filtration column equilibrated with 1×PBS. Fractions containing the HLA-DQ7.5/CLIP peptide complex were then pooled and stored at −80 degrees C.

1.2. Expression and Purification of Recombinant HLA-DQ2.5/Invariant Chain Complex

The sequences used for expression and purification are: HLA-DQA1*0501 (Protein Data Bank accession code 4OZG), HLA-DQB1*0201 (Protein Data Bank accession code 4OZG), invariant chain (76-295) (GenBank accession No. NM_001025159), all of which have a CAMPATH-1H signal sequence: MGWSCIILFLVATATGVHS (SEQ ID NO: 99). HLA-DQA1*0501 has C47S mutation, GGGG linker (SEQ ID NO: 100) and c-fos leucine zipper sequence (PNAS, 1998 Sep. 29; 95(20): 11828-33) on the C-terminus of HLA-DQA1*0501. HLA-DQB1*0201 has GGGGG linker (SEQ ID NO: 102) and c-jun leucine zipper sequence (PNAS, 1998 Sep. 29; 95(20): 11828-33), and 8× His-tag on the C-terminus of HLA-DQB1*0201. Invariant chain (76-295) has a Flag-tag, GCN4 variant amino acid sequence (Science. 1993 Nov. 26; 262(5138):1401-7), and GGGGS linker (SEQ ID NO: 102) on the N-terminus of invariant chain (76-295). A recombinant HLA-DQ2.5/invariant chain complex was expressed transiently using FreeStyle293-F cell line. Conditioned media expressing the recombinant HLA-DQ2.5/invariant chain complex was incubated with an IMAC resin, followed by elution with imidazole. Fractions containing the recombinant HLA-DQ2.5/invariant chain complex were collected and subsequently subjected to a Superose 6 gel filtration column (GE healthcare) equilibrated with 1×PBS. Fractions containing the recombinant HLA-DQ2.5/invariant chain complex were then pooled and stored at −80 degrees C.

1.3. Expression and Purification of Recombinant TCRs

The sequences used for expression and purification are: S2 TCR alpha chain (Protein Data Bank accession code 4OZI), S2 TCR beta chain (Protein Data Bank accession code 4OZI), D2 TCR alpha chain (Protein Data Bank accession code 4OZG), D2 TCR beta chain (Protein Data Bank accession code 4OZG). S2 TCR alpha chain has a CAMPATH-1H signal sequence: MGWSCIILFLVATATGVHS (SEQ ID NO: 99), and BAP sequence (BMC Biotechnol. 2008; 8: 41), 8× His-tag on the C-terminus of S2 TCR alpha chain. S2 TCR beta chain has a CAMPATH-1H signal sequence: MGWSCIILFLVATATGVH (SEQ ID NO: 108), and Flag-tag on the C-terminus of S2 TCR beta chain. D2 TCR alpha chain has a signal sequence derived from rat serum albumin: MKWVTFLLLLFISGSAFS (SEQ ID NO: 109), and BAP sequence (BMC Biotechnol. 2008; 8: 41), 8× His-tag on the C-terminus of D2 TCR alpha chain. D2 TCR beta chain has a signal sequence derived from rat serum albumin: MKWVTFLLLLFISGSAFS (SEQ ID NO: 109), and Flag-tag on the C-terminus of D2 TCR beta chain.

Recombinant soluble TCR protein was expressed transiently using FreeStyle293-F cell line. Conditioned media expressing TCR protein was applied to a column packed with anti-Flag M2 affinity resin (Sigma) and eluted with Flag peptide (Sigma). Fractions containing TCR protein were collected and subsequently applied to a column packed with an IMAC resin, followed by elution with imidazole. Fractions containing TCR protein were collected and subsequently subjected to a Superdex 200 gel filtration column equilibrated with 1×PBS. Fractions containing TCR protein were then pooled and stored at −80 degrees C. The purified TCR protein was biotinylated using BirA, then combined with PE-labeled streptavidin (BioLegend) to form tetrameric TCR protein.

### Example 2

2.1 Establishment of D2 TCR-Expressing J.RT3-T3.5 Cell Lines

D2 TCR alpha chain cDNA (SEQ ID NO: 110) was inserted into the expression vector pCXND3 (WO2008/156083). D2 TCR beta chain cDNA (SEQ ID NO: 111) was inserted into the expression vector pCXZD1 (US2009/0324589). The linearized D2 TCR alpha chain—pCXND3 and D2 TCR beta chain—pCXZD1 (1500 ng each) were simultaneously introduced into J.RT-T3.5 cell line by electroporation (LONZA, 4D-Nucleofector X). Transfected cells were then cultured in media containing Geneticin and Zeocin, after which sorting was performed to obtain a high-expressing cell population using AriaIII (Becton Dickinson). Single cell cloning was then performed to obtain cells that highly expressed the desired D2 TCR molecule.

2.2 Establishment of Ba/F3 Cell Lines Expressing HLA-DQ2.5, HLA-DQ2.5/gliadin peptide, HLA-DQ2.5/CLIP peptide, HLA-DQ2.2, HLA-DQ7.5, HLA-DQ8, HLA-DQ5.1, HLA-DQ6.3, HLA-DQ7.3, HLA-DR, and HLA-DP HLA-DQA1*0501 cDNA (IMGT/HLA accession No. HLA00613), HLA-DQA1*0201 cDNA (IMGT/HLA accession No. HLA00607), HLA-DQA1*0505 cDNA (IMGT/HLA accession No. HLA00619), HLA-DQA1*0301 cDNA (IMGT/HLA accession No. HLA00608), HLA-DQA1*0101 cDNA (IMGT/HLA accession No. HLA00601), HLA-DQA1*0103 cDNA (IMGT/HLA accession No. HLA00604), HLA-DQA1*0303 cDNA (IMGT/HLA accession No. HLA00611), HLA-DRA1*0101 cDNA (GenBank accession No. NM_019111.4), or HLA-DPA1*0103 cDNA (IMGT/HLA accession No. HLA00499), was inserted into the expression vector pCXND3 (WO2008/156083).

HLA-DQB1*0201 cDNA (IMGT/HLA accession No. HLA00622), HLA-DQB1*0202 cDNA (IMGT/HLA accession No. HLA00623), HLA-DQB1*0301 cDNA (IMGT/HLA accession No. HLA00625), HLA-DQB1*0302 cDNA (IMGT/HLA accession No. HLA00627), HLA-DQB1*0501 cDNA (IMGT/HLA accession No. HLA00638), HLA-DQB1*0603 cDNA (IMGT/HLA accession No. HLA00647), HLA-DRB1*0301 cDNA (IMGT/HLA accession No. HLA00671), or HLA-DPB1*0401 cDNA (IMGT/HLA accession No. HLA00521) was inserted into the expression vector pCXZD1 (US/20090324589). HLA-DQB1*0201 for the HLA-DQ2.5/33mer gliadin peptide complex has 33mer gliadin peptide sequence: LQLQPFPQPELPYPQPELPYPQPELPYPQPQPF (SEQ ID NO: 101), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/CLIP peptide complex has CLIP peptide sequence: KLPKPPKPVSKMRMATPLLMQALPMGALP (SEQ ID NO: 103), and factor X cleavage linker: (Acta Crystallogr Sect F Struct Biol Cryst Commun. 2007 Dec. 1; 63 (Pt 12): 1021-1025.) on the N-terminus of HLA-DQB1*0201.

1000 ng each of the linearized HLA-DQA1*0501-pCXND3 and HLA-DQB1*0201-pCXZD1, and 500 ng each of the linearized HLA-DQA1*0201-pCXND3 and HLA-DQB1*0202-pCXZD1, HLA-DQA1*0505-pCXND3 and HLA-DQB1*0301-pCXZD1, HLA-DQA1*0301-pCXND3 and HLA-DQB1*0302-pCXZD1, HLA-DQA1*0101-pCXND3 and HLA-DQB1*0501-pCXZD1, HLA-DQA1*0103-pCXND3 and HLA-DQB1*0603-pCXZD1, HLA-DQA1*0303-pCXND3 and HLA-DQB1*0301-pCXZD1, HLA-DRA1*0101-pCXND3 and HLA-DRB1*0301-pCXZD1, HLA-DPA1*0103-pCXND3 and HLA-DPB1*0401-pCXZD1, HLA-DQA1*0501-pCXND3 and HLA-DQB1*0201 for HLA-DQ2.5/33mer gliadin peptide -pCXZD1, HLA-DQA1*0501-pCXND3 and HLA-DQB1*0201 for HLA-DQ2.5/CLIP peptide -pCXZD1 were simultaneously introduced into mouse IL-3-dependent pro-B cell-derived cell line Ba/F3 by electroporation (LONZA, 4D-Nucleofector X). Transfected cells were then cultured in media containing Geneticin and Zeocin, after which sorting was performed to obtain a high-expressing cell population using AriaIII (Becton Dickinson). Single cell cloning was then performed to obtain cells that highly expressed the desired HLA molecules. Established each cell lines were named Ba/F3-HLA-DQ2.5 (HLA-DQA1*0501, HLA-DQB1*0201), HLA-DQ2.2 (HLA-DQA1*0201, HLA-DQB1*0202), HLA-DQ7.5 (HLA-DQA1*0505, HLA-DQB1*0301), Ba/F3-HLA-DQ8 (HLA-DQA1*0301, HLA-DQB1*0302), HLA-DQ5.1 (HLA-DQA1*0101, HLA-DQB1*0501), HLA-DQ6.3 (HLA-DQA1*0103, HLA-DQB1*0603), HLA-DQ7.3 (HLA-DQA1*0303, HLA-DQB1*0301), Ba/F3-HLA-DR (HLA-DRA1*0101, HLA-DRB1*0301), Ba/F3-HLA-DP (HLA-DPA1*0103, HLA-DPB1*0401), HLA-DQ2.5/33mer gliadin peptide (HLA-DQA1*0501, HLA-DQB1*0201 for HLA-DQ2.5/33mer gliadin peptide), HLA-DQ2.5/CLIP peptide (HLA-DQA1*0501, HLA-DQB1*0201 for HLA-DQ2.5/CLIP peptide)

### Example 3

Generation of Anti-DQ2.5 Antibodies

Anti-DQ2.5 antibodies were prepared, selected and assayed as follows:

NZW rabbits were immunized intradermally with the HLA-DQ2.5/33mer gliadin peptide complex. Four repeated doses were given over a 2-month period followed by blood and spleen collection. For B-cell selection, a biotinylated HLA-DQ5.1/DBY peptide complex, biotinylated HLA-DQ8/gliadin peptide complex, and Alexa Fluor 488-labeled HLA-DQ2.5/33mer gliadin peptide complex were prepared. B-cells that can bind to HLA-DQ2.5 but not HLA-DQ5.1 or HLA-DQ8 were stained with the labeled proteins described above, sorted using a cell sorter and then plated and cultured according to the procedure described in WO2016098356A1. After cultivation, the B-cell culture supernatants were collected for further analysis and the B-cell pellets were cryopreserved.

Specific binding to the HLA-DQ2.5/33mer gliadin peptide complex was evaluated and non cross-reactivity to the HLA-DQ5.1/DBY peptide complex and the HLA-DQ8/gliadin peptide complex was confirmed by ELISA using the B cell culture supernatants. The results showed that 880 B cell lines exhibited specific binding to the HLA-DQ2.5/33mer gliadin peptide complex.

In order to evaluate the cross-reactivity to the HLA-DQ2.2/CLIP peptide complex and the HLA-DQ7.5/CLIP peptide complex, ELISA was conducted using the selected 880 B cell supernatants described above. In addition, neutralizing activity was checked by neutralizing assay using the selected 880 B cell supernatants.

The procedure of neutralizing assay was in accordance with the AlphaLISA neutralizing assay (HLA-DQ2.5/33mer gliadin peptide—D2 TCR) described below. B cells with high neutralizing activities were preferred and selected for cloning.

The RNAs of 188 B cell lines with desired binding specificities were purified from the cryopreserved cell pellets using the ZR-96 Quick-RNA kits (ZYMO RESEARCH, Cat No. R1053). These were named DQN0189-0376. DNAs encoding antibody heavy-chain variable regions in the selected cell lines were amplified by reverse transcription PCR and recombined with a DNA encoding the F1332m heavy-chain constant region (SEQ ID NO: 97). DNAs encoding antibody light-chain variable regions were also amplified by reverse transcription PCR and recombined with a DNA encoding the hk0MC light-chain constant region (SEQ ID NO: 98). Cloned antibodies were expressed in Freestyle™ 293-F Cells (Invitrogen) and purified from culture supernatants. Through further evaluation described below, twelve clones (DQN0223hh, DQN0235ee, DQN0303hh, DQN0333hh, DQN0282ff, DQN0356bb, DQN0344xx, DQN0334bb, DQN0225dd, DQN0271hh, DQN0324hh and DQN0370hh) were selected based on binding ability, specificity and functionality. Two clones (DQN0089ff and DQN0139bb) were used as assay controls. The VH and VL sequences of these antibodies are shown in Tables 2 and 3. The sequence ID numbers of VH, VL, HCDRs and LCDRs of these antibodies are listed in Table 4. The sequences of these CDRs are shown in Tables 2 and 3.

For example, “DQN0223Hh” in Table 2 and “DQN0223Lh” in Table 3 respectively show the H-chain and L-chain region sequences of the DQN0223hh antibody. The same applies to the other antibodies such as DQN0235ee, DQN0303hh, DQN0333hh, DQN0282ff, DQN0356bb, DQN0344xx, DQN0334bb, DQN0225dd, DQN0271hh, DQN0324hh, DQN0370hh, DQN0089ff, DQN0177aa and DQN0139bb.

The H-chain sequences of the antibodies are shown in Table 2. The L-chain sequences of the antibodies are shown in Table 3. The sequence ID numbers of the regions of the antibodies are shown in Table 4.

TABLE 2

| | F R 1 | C D R 1 | F R 2 | C D R 2 |
|---|---|---|---|---|
| | 0 1 2 3 | 3 | 3 4 | 5 6 |
| Name | 1 2 3 4 5 6 7 8 9 0 1 2 3 4 5 6 7 8 9 0 1 2 3 4 5 8 7 8 9 0 | 1 2 3 4 5 a b | 6 7 8 9 0 1 2 3 4 5 6 7 8 9 | 0 1 2 a b c 3 4 5 6 7 8 9 0 1 2 3 4 5 |
| DQN0223Hh | Q Q Q L E E S G G G L V K P G G T L T L T C K A S R I D F S | T Y Y Y I C - | W V R Q S P W K G L E W I A | C I Y S - - G D G Y T Y Y P S W A K G |
| DQN0235He | Q Q Q L E E S G G G L V K P G G T L T L T C K A G I D F S T | Y Y Y M C - - | W V R Q A P G K R P E W I A | C I F D - - G D G S T Y Y A S W V N G |
| DQN0303Hh | Q Q H L E E S G G G L V K P G G T L T L T C T A S G I D F S | S Y Y Y M C - | W V R Q A P G K G L E W I A | C I Y T - - G D G S T Y Y A S W V N G |
| DQN0333Hh | Q - S V E E S G G R L V M P G G S L T L T C T A S G F S L S | S Y Y M N - - | W V R Q A P G K G L E W I G | V I Y - - - S T D I T D Y A T W A T G |
| DQN0282Hf | Q - S V E E S G G R L V T P G T P L T L S C K A S G F D F S | I Y Y M S - - | W V R Q A P G K G L E W I G | V I N S - - G G G V T W Y A S W V K G |
| DQN0356Hb | Q E Q L V E S G G G L V K P E G S L T L T C T A S G F S F S | S D Y S V C - | W V R Q A P G K G L E W I G | C I Y - - T G S D I T Y Y A S W A K G |
| DQN0344Hx | Q - S L E E S G G G L V K P G G T L T L T C T A S G F S F S | S S Y W M C - | W V R Q A P G K G L E W V A | C V Y G - - G S D T T Y Y A S W T K G |
| DQN0334Hb | Q Q Q L E E S G G D L V Q P E G S L T L T C K A S G F D F S | F N A M C - - | W V R Q A P G R G P E W I A | C I Y N - - G N G S T Y Y A S W A K G |
| DQN0139Hb | Q - S L E E S G G R L V T P G T A L T L T C T V S G F S L S | S Y A M G - - | W V R Q A P G K G L E Y I G | W T S - - - P G D S A Y Y A S W T K G |
| DQN0177Ha | Q - S V E E S G G R L V T P G T P L T L T C T V S G F S L S | S Y A M S - - | W V R Q A P G K G L E W I G | V I S - - - Y S G R T Y D A S W A K G |
| DQN0089Hf | Q E Q V V E Y G G D L V Q P E G S L T L T C K A S G L D F S | S T Y Y M C - | W V R Q A P G K G L E W I G | C I Y G G - S S D S T Y Y A S W A K G |
| IC17H | Q V Q L Q Q S G P Q L V R P G A S V N I S C K A S G Y S F T | S Y W M H - - | W V N Q R P G Q G L E W I G | M I D P - - S Y S E T R L N Q K F K D |
| DQN0225Hd | Q - S L E E S G G G L V K P G G T L T L T C T A S G F S F S | S A Y Y M C - | W V R Q A P G K G L E W I A | C I Y V - - G S G S T Y Y A S W A Q G |
| DQN0271Hh | Q - S L E E S G G G L V K P G A S L T L T C K A S G F S F S | S G Y Y M C - | W V R Q A P G K G L E W I A | C I Y A G - S P D L T Y Y A T W A K G |
| DQN0324Hh | Q - S L E E S G G D L V K P G A S L A L T C T A S G F S L I | N N Y Y M C - | W V R Q A P G K G L E W I A | C I Y A G - L G G W T Y Y A S W A K G |
| DQN0370Hh | Q - S L E E S G G G L V K P G G P L T L T C T A S G F S F S | S A Y Y M C - | W V R Q A P G K G L E W I A | C I Y V G - - S G S T Y Y T S W A K G |

| | F R 3 | C D R 3 | F R 4 |
|---|---|---|---|
| | 6 7 8 9 | 9 1 0 | 1 1 |
| Name | 6 7 8 9 0 1 2 3 4 5 6 7 8 9 0 1 2 a b c 3 4 5 6 7 8 9 0 1 2 3 4 | 5 6 7 8 9 0 a b c d e f g h i j k l 1 2 | 3 4 5 6 7 8 9 0 1 2 3 |
| DQN0223Hh | R F S I S K T S S T - T V T L Q V T S L T A A D T A T Y F C A R | S Y G G S S A S A Y F - - - - - - - T L | W G P G T Q V T V S S |
| DQN0235He | R F T I S K A S S T - T V T L R M T S L T A A D T A T Y F C A R | S A G G S S S F G Y F - - - - - - - N L | W G P G T L V T V S S |
| DQN0303Hh | R F T I S R S T S L N T V T L Q L N G L A A A D T A T Y F C A R | S Y G A S S A I G Y F - - - - - - - N L | W G P G T L V T V S S |
| DQN0333Hh | R F T I S K T S T - - T V D L K M T S L T A A D T A T Y F C A R | E L S P T T S G Y I A - - - - - - - Y L | W G P G T L V T V S S |
| DQN0282Hf | R F T I S K T S T - - T V D L K I T S P T T E D T A T Y F C A R | L G D S N H Y - - - - - - - - - - - D L | W G P G T L V T V S S |
| DQN0356Hb | R F T I S K T S S T - T V T L Q M T S L T A A D T A T W F C A R | S W N G Y G G Y A - - - - - - - - - D L | W G P G T L V T V S S |
| DQN0344Hx | R F T I S K G S S T - T V A L Q V T S L T A A D T A T Y F C A R | D P L N Y Y Y Y G E L - - - - - - - N L | W G P G T L V T V S S |
| DQN0334Hb | R F T I S K T S S T - S V T L Q M T S L T A A D T A T Y F C A R | G G Y G L G Y A G Y G D V N Y F - - N L | W G P G T L V T V S S |
| DQN0139Hb | R F T I S R T S T - - T V E L K I T S P T T E D T A T Y F C A R | D A A Y I G H W A F - - - - - - - - N L | W G P G T L V T V S S |
| DQN0177Ha | R F T I S K T S T - - T V D L K I T S P T T E D T A T Y F C A R | V G D S Y G Y A Y A T V I F T Y H F N L | W G P G T L V T V S S |
| DQN0089Hf | R F T I S K T S P T - T V T L Q M T S L T A A D T A T H F C A T | Y D Y G A V G Y - - - - - - - - - - D L | W G P G T L V T V S S |
| IC17H | K A T L T V D K S S S T A Y M Q L S S P T S E D S A V Y Y C A L | Y G N Y F - - - - - - - - - - - - - D Y | W G Q G T T L T V S S |
| DQN0225Hd | R C T I S K A S S T - T V T L Q M T S L T A A D T A T Y F C A R | D P L I Y Y Y Y G E L - - - - - - - N L | W G P G T L V T V S S |
| DQN0271Hh | R L T I S T T S S - T T V T L Q L T S L T A A D T A T Y F C A R | D P G N W G G Y D D V - - - - - - - R L | W G P G T L V T V S S |
| DQN0324Hh | R F T I S K T S S - T T V T L Q M S S L T V A D A A T Y F C A R | A A T T Y Y H F - - - - - - - - - - N L | W G P G T L V T V S S |
| DQN0370Hh | R F T I S K V S S - T T M T L Q M T S L T A A D T A T Y F C A R | D P L I Y Y Y Y G E L - - - - - - - N L | W G P G T L V T V S S |

TABLE 3

| | F R 1 | C D R 1 |
|---|---|---|
| | 0 1 2 | 2 3 |
| Name | 1 2 3 4 5 6 7 8 9 0 1 2 3 4 5 6 7 8 9 0 1 2 3 | 4 5 6 7 a b c d e f 8 9 0 1 2 3 4 |
| DQN0223Lh | A V V L T Q T A S P V S A P V G G T V T I K C | Q A S Q - - - - - - S I G S N L G |
| DQN0235Le | A Y D M T Q T P S S V S A A V G G T V T I K C | Q A S Q S - - - - - I S S S Y L A |
| DQN0303Lh | D V V M T Q T P S S K S A A V G G T V T I K C | Q A S Q - - - - - - N I Y S N L A |
| DQN0333Lh | A Y D M T Q T P A S V E V A V G G T V T I K C | Q A S Q - - - - - - S I S S Y L A |
| DQN0282Lf | A Y D M T Q T P S S V S A A V G G T V T I K C | Q A S E - - - - - - T I S S Y L A |
| DQN0356Lb | A Y D M A Q T P A S V E V A V G G T V T I K C | Q A S Q - - - - - - S I D N Y L A |
| DQN0344Lx | A V V L T Q T A S P V S A A V G G T V T I R C | Q A T E - - - - - - N I Y S G L A |
| DQN0334Lb | A V V L T Q T A S P V S A P V G G T V T I K C | Q A S E - - - - - - D I Y F L L A |
| DQN0139Lb | D V V M T Q T P A S V S A P V G G T V T I N C | Q A S E - - - - - - S I Y S N L A |
| DQN0177La | D P V L T Q T P S S A S E P V G G T V T I K C | Q A S E - - - - - - S I S S S L A |
| DQN0089Lf | E V V V T Q T P A S V E V A V G G T V T I K C | Q A S Q - - - - - - N I S P Y L S |
| IC17L | D I Q M T Q S S S S F S V S L G D R V T I T C | K A S E - - - - - - D I Y N R L A |
| DQN0225Ld | A V V L T Q T A S P V S A P V G G T V T I K C | Q A S E - - - - - - D I S S N L A |
| DQN0271Lh | A Q V L T Q T P S P V S A A V G G T V T I S C | Q S S Q S - - - - - I Y S N Y L S |
| DQN0324Lh | A V V L T Q T E S P V S A A V G D T V T I K C | Q A S Q S I G - - - - - - N A L A |
| DQN0370Lh | A V V L T Q T A S P V S A P V G G T V T I K C | Q A S E D I Y S - - - - - - N L A |

| | F R 2 | C D R 2 |
|---|---|---|
| | 3 4 | 5 |
| Name | 5 6 7 8 9 0 1 2 3 4 5 6 7 8 9 | 0 1 2 3 4 5 6 |
| DQN0223Lh | W Y Q Q K P G Q P P K L L I Y | G A S A L A S |
| DQN0235Le | W Y H Q K P G Q S P K L L I Y | S A S T L A S |
| DQN0303Lh | W Y Q Q K P G Q R P K L L I Y | E A S T L A S |
| DQN0333Lh | W Y Q Q K P G Q P P K L L I Y | S A S T L A S |
| DQN0282Lf | W Y Q Q K P G Q R P Q L L I Y | D A S N L A S |
| DQN0356Lb | W Y Q Q K P G Q P P K L L I Y | W A S T L A S |
| DQN0344Lx | W Y Q Q K P G Q P P K V L I Y | Y V S T L A S |
| DQN0334Lb | W Y R Q K P G Q P P K L L I Y | G A S T L A S |
| DQN0139Lb | W Y Q Q K P G Q P P K L L I Y | G A S T L E S |
| DQN0177La | W Y Q Q K P G Q R P K L L I Y | D A S K L A S |
| DQN0089Lf | W Y Q Q K P G Q P P K L L I Y | K A S T L A S |
| IC17L | W Y Q Q K P G N A P R L L I S | G A T S L E T |
| DQN0225Ld | W Y Q Q K P G Q R P K V L I Y | S A S T L A S |
| DQN0271Lh | W F Q Q K T G Q P P K L L I Y | R A S T L A S |
| DQN0324Lh | W Y Q Q K P G Q P P K L L I Y | D A S T L A S |
| DQN0370Lh | W Y Q Q K P G Q R P K V L I Y | S A S T L A S |

| | F R 3 |
|---|---|
| | 5 6 7 8 |
| Name | 7 8 9 0 1 2 3 4 5 6 7 8 9 0 1 2 3 4 5 6 7 8 9 0 1 2 3 4 5 6 7 8 |
| DQN0223Lh | G V P S R F K G S G S G T Q F T L T I N D L E C A D A A T Y Y C |
| DQN0235Le | G V P S R F K G S G S G T E F T L T I S D L A C A D A A T Y Y C |
| DQN0303Lh | G V P S R F R G S G S G T E F T L T I S D L E C D D A A T Y Y C |
| DQN0333Lh | G V P S R F K G S G S G T Q F T L T V I D V Q C D D A A T Y Y C |
| DQN0282Lf | G V P S R F K G S G S G T E F T L T I S D L E C A D A A T Y Y C |
| DQN0356Lb | G V S S R F K G S G S G T Q F S L T I S D L E C A D A A T Y Y C |
| DQN0344Lx | G V P S R F K G S G S G T E Y T L T I S G V Q C D D A A T Y Y C |
| DQN0334Lb | G V P S R F K G S G S G T E Y T L T I S D L E C A D A A T Y Y C |
| DQN0139Lb | G V P S R V K G S G Y G T Q F T L T I S D L E C A D A A T Y Y C |
| DQN0177La | G V P S R F K G S G S G T E F T L T I S G V Q C D D A A T Y Y C |
| DQN0089Lf | G V S S R F K G S G S G T Q F T L T I S D L E C A D A A T Y Y C |
| IC17L | G V P S R F S G S G S G K D Y T L S I T S L Q T E D V A T Y Y C |
| DQN0225Ld | G V S S Q F K G S G S G T Q F T L T I S D L E C A D A A T Y Y C |
| DQN0271Lh | G V P S R F K G S G S G T E F T L T I S G V E C D D A A T Y Y C |
| DQN0324Lh | G V P S R F K G S G S G T Q F T L T I S D L E C A D A A T Y Y C |
| DQN0370Lh | G V S S R F K G S R S G T E Y T L S I T D L G C A D A A T Y Y C |

| | C D R 3 | F R 4 |
|---|---|---|
| | 9 | 1 0 |
| Name | 9 0 1 2 3 4 5 a b c d e f 6 7 | 8 9 0 1 2 3 4 5 6 7 |
| DQN0223Lh | Q G Y Y Y S S S A E W - - H S | F G G G T K V E I K |
| DQN0235Le | Q S Y Y Y F S S N A W - - H T | F G G G T K V E I K |
| DQN0303Lh | Q N Y Y Y T S S N D F - - Y T | F G G G T K V E I K |
| DQN0333Lh | Q Q T Y S G K N V I - - - N T | F G G G T K V E I K |
| DQN0282Lf | Q T H Y Y I T S - - - - - T T | F G G G T K V E I K |
| DQN0356Lb | Q Q Y Y S N S N V V - - - N T | F G G G T K V E I K |
| DQN0344Lx | Q T Y H D I S N - - - - - V T | F G G G T K V E I K |
| DQN0334Lb | Q S Y W Y G D S G P - - - N T | F G G G T K V E I K |
| DQN0139Lb | Q Q Y Y G S S S T A - - - F T | F G G G T K V E I K |
| DQN0177La | Q H G L S S G S T N R - - C A | F G G G T K V E I K |
| DQN0089Lf | Q N N Y G V S I N Y G - - H T | F G G G T K V E I K |
| IC17L | Q Q Y W S T P - - - - - - Y T | F G G G T K L E V K |
| DQN0225Ld | Q S Y Y D I S S - - - - - V T | F G G G T K V E I K |
| DQN0271Lh | Q G Y Y S G A I - - - - - W T | F G G G T K V E I K |
| DQN0324Lh | Q S Y D Y G S S A D T - - Y T | F G G G T K V E I K |
| DQN0370Lh | Q A Y Y D I G G - - - - - V T | F G G G T K V E I K |

TABLE 4

| Antibody | SEQ ID NO: | | | | | | | |
|---|---|---|---|---|---|---|---|---|
| Name | VH | HCDR1 | HCDR2 | HCDR3 | VL | LCDR1 | LCDR2 | LCDR3 |
| DQN0223hh | 1 | 13 | 25 | 37 | 49 | 61 | 73 | 85 |
| DQN0235ee | 2 | 14 | 26 | 38 | 50 | 62 | 74 | 86 |
| DQN0303hh | 3 | 15 | 27 | 39 | 51 | 63 | 75 | 87 |
| DQN0333hh | 4 | 16 | 28 | 40 | 52 | 64 | 76 | 88 |
| DQN0282ff | 5 | 17 | 29 | 41 | 53 | 65 | 77 | 89 |
| DQN0356bb | 6 | 18 | 30 | 42 | 54 | 66 | 78 | 90 |
| DQN0344xx | 7 | 19 | 31 | 43 | 55 | 67 | 79 | 91 |
| DQN0334bb | 8 | 20 | 32 | 44 | 56 | 68 | 80 | 92 |
| DQN0139bb | 9 | 21 | 33 | 45 | 57 | 69 | 81 | 93 |
| DQN0225dd | 142 | 146 | 150 | 154 | 158 | 162 | 166 | 170 |
| DQN0271hh | 143 | 147 | 151 | 155 | 159 | 163 | 167 | 171 |
| DQN0324hh | 144 | 148 | 152 | 156 | 160 | 164 | 168 | 172 |
| DQN0370hh | 145 | 149 | 153 | 157 | 161 | 165 | 169 | 173 |
| DQN0177aa | 10 | 22 | 34 | 46 | 58 | 70 | 82 | 94 |
| DQN0089ff | 11 | 23 | 35 | 47 | 59 | 71 | 83 | 95 |
| IC17 | 12 | 24 | 36 | 48 | 60 | 72 | 84 | 96 |

### Example 4

Characterization of Anti-HLA-DQ2.5 Antibodies

4.1. Binding analysis of the antibodies to HLA-DQ2.5, HLA-DQ2.2, and HLA-DQ7.5 FIGS. 1 to 5 show the binding of the anti-HLA-DQ antibodies to a panel of multiple WIC class II-expressing Ba/F3 cell lines as determined by FACS. The binding of anti-HLA-DQ antibodies to Ba/F3-HLA-DQ2.5 (expressing HLA-DQ2.5), Ba/F3-HLA-DQ2.5/33mer gliadin peptide (expressing HLA-DQ2.5/33mer gliadin peptide), Ba/F3-HLA-DQ2.5/CLIP peptide (expressing HLA-DQ2.5/CLIP peptide), Ba/F3-HLA-DQ2.2 (expressing HLA-DQ2.2), and Ba/F3-HLA-DQ7.5 (expressing HLA-DQ7.5) was tested. 10 microgram/mL of anti-HLA-DQ antibodies were incubated with each cell line for 30 minutes at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 20 minutes at 4 degrees C., and this was washed with FACS buffer. Data acquisition was performed on LSRFortessa X-20 (Becton Dickinson), followed by analysis using the FlowJo software (Tree Star) and GraphPad Prism software (GraphPad).

FIG. 1 shows that all of the anti-HLA-DQ2.5 antibodies produced in Example 3, i.e., DQN0223hh, DQN0235ee, DQN0303hh, DQN0333hh, DQN0282ff, DQN0356bb, DQN0344xx, DQN0334bb, DQN0089ff, and DQN0139bb have binding activity to the HLA-DQ2.5/gliadin peptide complex. IC17 shows the level of a negative control (background) in which the anti-HLA-DQ2.5 antibody was not added in the experiments. The same applies to the other Figures.

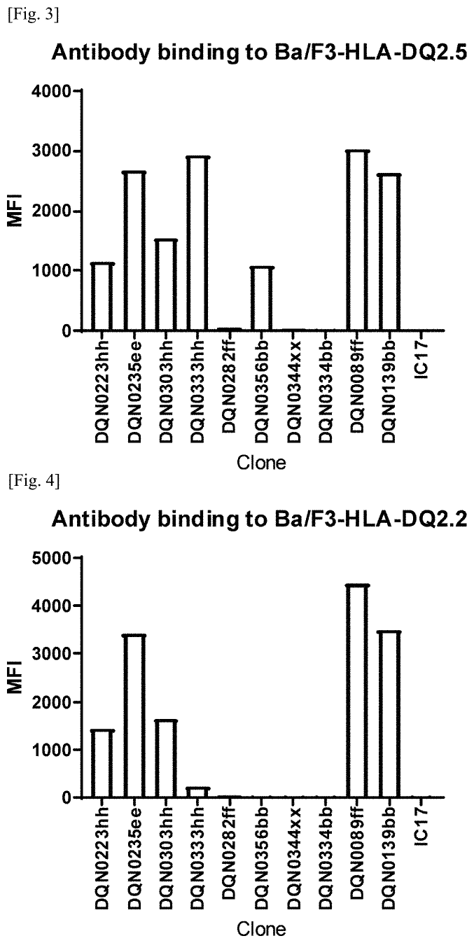

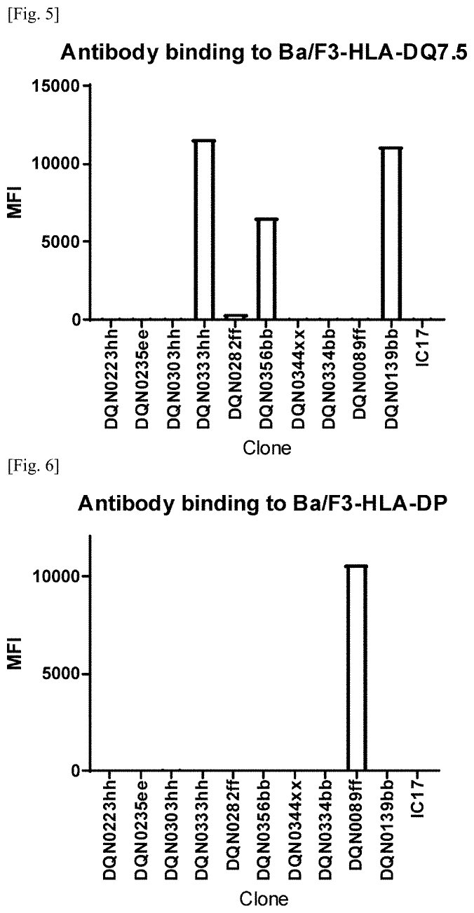

FIG. 2 shows that DQN0223hh, DQN0235ee, DQN0303hh, DQN0333hh, DQN0282ff, DQN0356bb, DQN0089ff, and DQN0139bb have binding activity to the HLA-DQ2.5/CLIP peptide complex, whereas DQN0344xx and DQN0334bb have substantially no binding activity to the same. FIG. 3 shows that DQN0223hh, DQN0235ee, DQN0303hh, DQN0333hh, DQN0356bb, DQN0089ff, and DQN0139bb have binding activity to HLA-DQ2.5 which is not in the form of a complex with gliadin peptide or CLIP peptide, whereas DQN0282ff, DQN0344xx, and DQN0334bb have substantially no binding activity to the same. FIG. 4 shows that DQN0223hh, DQN0235ee, DQN0303hh, DQN0089ff, and DQN0139bb have binding activity to HLA-DQ2.2, whereas DQN0333hh, DQN0282ff, DQN0356bb, DQN0344xx, and DQN0334bb have substantially no binding activity to the same. FIG. 5 shows that DQN0333hh, DQN0356bb, and DQN0139bb have binding activity to HLA-DQ7.5, whereas DQN0223hh, DQN0235ee, DQN0303hh, DQN0282ff, DQN0344xx, DQN0334bb, and DQN0089ff have substantially no binding activity to the same.

4.2. Binding analysis of the antibodies to HLA-DQ8/5.1/6.3/7.3 and HLA-DR/DP HLA-DQ5.1/6.1/6.3/6.4/7.3/8 are known to be major HLA-DQ alleles among European americans (Tissue Antigens. 2003 October; 62(4):296-307). In view of high sequence similarity among HLA-DQ6.1/6.3/6.4, the HLA-DQ6.3 allele was selected as a representative.

FIGS. 6 to 11 show the binding of the anti-HLA-DQ antibodies to multiple MHC class II-expressing Ba/F3 cell lines as determined by FACS. The binding of the anti-HLA-DQ antibodies to Ba/F3-HLA-DQ8, BaF3-HLA-DQ5.1, BaF3-HLA-DQ6.3, Ba/F3-HLA-DQ7.3, and Ba/F3-HLA-DR, Ba/F3-HLA-DP was tested. 20 microgram/mL of anti-HLA-DQ antibodies were incubated with each cell line for 30 minutes at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 20 minutes at 4 degrees C. and washed with FACS buffer. Data acquisition was performed on LSRFortessa X-20 (Becton Dickinson), followed by analysis using the FlowJo software (Tree Star) and GraphPad Prism software (GraphPad).

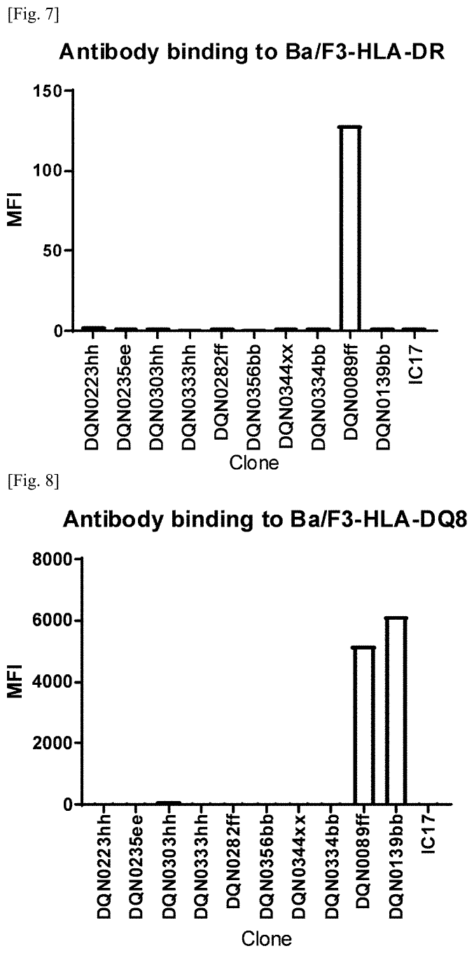

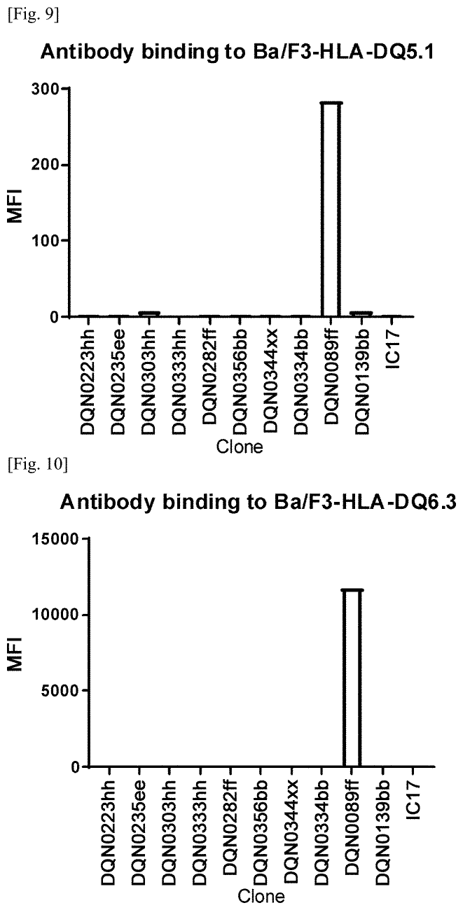

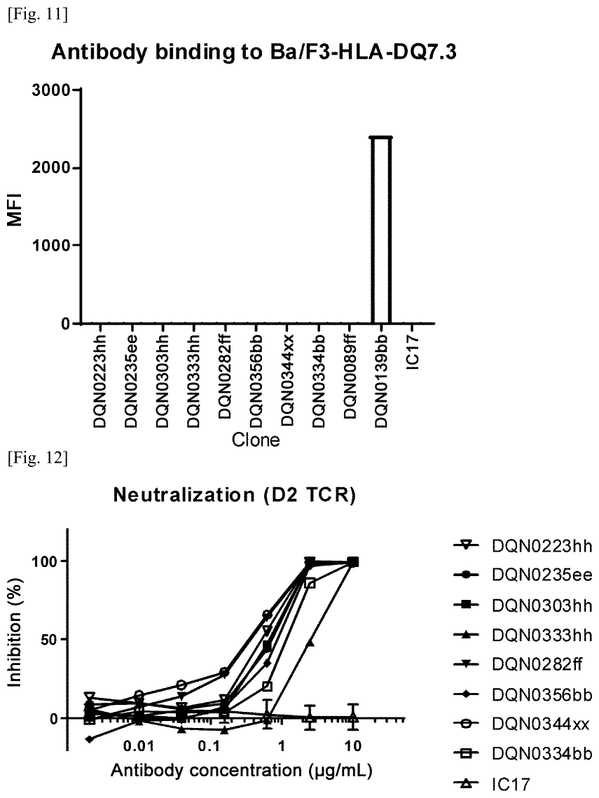

FIGS. 6 and 7 show that DQN0089ff has binding activity to HLA-DP/DR, whereas the other antibodies have substantially no binding activity to the same. FIG. 8 shows that DQN0089ff and DQN0139bb have binding activity to HLA-DQ8, whereas the other antibodies have substantially no binding activity to the same. FIGS. 9 and 10 show that DQN0089ff has binding activity to HLA-DQ5.1/6.3, whereas the other antibodies have substantially no binding activity to the same. FIG. 11 shows that DQN0139bb has binding activity to HLA-DQ7.3, whereas the other antibodies have substantially no binding activity to the same.

4.3. Binding Analysis of the Antibodies to HLA-DQ2.5/33Mer Gliadin Peptide Complex

The affinity of anti-HLA-DQ antibodies to the HLA-DQ2.5/33mer gliadin peptide complex at pH 7.4 were determined at 37 degrees C. using Biacore 8K instrument (GE Healthcare). Anti-human Fc (GE Healthcare) was immobilized onto all flow cells of a CM4 sensor chip using amine coupling kit (GE Healthcare). All antibodies and analytes were prepared in ACES pH 7.4 containing 20 mM ACES, 150 mM NaCl, 0.05% Tween 20, and 0.005% NaN3. Each antibody was captured onto the sensor surface by anti-human Fc. Antibody capture levels were aimed at 200 resonance unit (RU). The recombinant HLA-DQ2.5/33mer gliadin peptide complex diluted by two-fold serial dilution to 50 to 800 nM was injected, and then subjected to dissociation. Sensor surface was regenerated after each cycle with 3M MgCl2. Binding affinity was determined by processing and fitting the data to 1:1 binding model using Biacore 8K Evaluation software (GE Healthcare).

The affinity of the anti-HLA-DQ2.5 antibodies for binding to the HLA-DQ2.5/33mer gliadin peptide complex is shown in Table 5.

These results demonstrate that the anti-HLA-DQ2.5 antibodies of the present invention bind to the HLA-DQ2.5 in the presence of the 33mer gliadin peptide, i.e., bind to the HLA-DQ bound by the 33mer gliadin peptide.

TABLE 5

| Ab name | ka (M⁻¹s⁻¹) | kd (s⁻¹) | KD (M) |
|---|---|---|---|
| DQN0223hh | 1.29E+05 | 2.64E−02 | 2.05E−07 |
| DQN0235ee | 7.44E+04 | 1.68E−03 | 2.25E−08 |
| DQN0282ff | 6.22E+04 | 8.50E−04 | 1.37E−08 |
| DQN0303hh | 6.79E+04 | 3.96E−04 | 5.83E−09 |
| DQN0333hh | 1.39E+05 | 1.87E−03 | 1.34E−08 |
| DQN0334bb | 8.93E+04 | 5.61E−04 | 6.29E−09 |
| DQN0344xx | 1.75E+05 | 1.68E−03 | 9.57E−09 |
| DQN0356bb | 6.04E+04 | 2.07E−02 | 3.42E−07 |

4.4. Neutralizing assay of the antibodies AlphaLISA neutralizing assay (HLA-DQ2.5/33mer gliadin peptide—D2 TCR) The neutralizing activity of anti-HLA-DQ antibodies towards the binding of the HLA-DQ2.5/33mer gliadin peptide complex and D2 TCR was assessed using the AlphaLISA beads assay platform. 40 micro g/mL of Streptavidin-AlphaLISA acceptor beads (Perkin Elmer, AL125M) were immobilized with 10 nM of biotinylated HLA-DQ2.5/33mer gliadin peptide in alphascreen buffer at pH 7.4 (40 mM HEPES/NaOH (pH 7.4), 100 mM NaCl, 1 mM CaCl2, 0.1% BSA, 0.05% Tween-20) for 60 minutes at room temperature. Simultaneously, 80 micro g/mL of Streptavidin-Alphascreen donor beads (Perkin Elmer, 6760002) were immobilized with 2.5 nM of biotinylated D2 TCR in alphascreen buffer for 60 minutes at room temperature. Then, using a 384-well plate, 10 micro L of serially diluted anti-HLA-DQ antibodies were incubated with 5 micro L of the HLA-DQ2.5/33mer gliadin peptide-coated acceptor beads and 5 micro L of the D2 TCR-coated donor beads for 60 minutes at room temperature. Alphascreen signal (counts per second, CPS) was measured by SpectraMax Paradigm (Molecular Devices), followed by analysis using GraphPad Prism software (GraphPad).

As shown in FIG. 12, the antibodies have neutralizing activity against the binding between gliadin-bound HLA-DQ2.5 and D2 TCR.

Beads neutralizing assay (HLA-DQ2.5/33mer gliadin peptide—S2 TCR) The neutralizing activity of the anti-HLA-DQ antibodies towards the binding of the HLA-DQ2.5/33mer gliadin peptide complex and S2 TCR was assessed using the Beads assay platform. Streptavidin-coated yellow particles (Spherotech, SVFB-2552-6K) were incubated in blocking buffer (2% BSA in PBS) for 30 minutes with shaking at room temperature. After centrifuging and aspirating supernatant, the soluble HLA-DQ2.5/33mer gliadin peptide complex was then added at 1.2×10⁴ beads/micro L of solution and immobilized for 60 minutes with shaking at room temperature on 96-well plates (Sigma Aldrich, Cat No. M2686). Final concentration of the HLA-DQ2.5/33mer gliadin peptide complex was 0.375 micro g/mL. The plates were washed with blocking buffer, and serially diluted anti-HLA-DQ antibodies were added and incubated for 60 minutes with shaking at room temperature. S2 TCR tetramer-PE was then added to HLA-DQ2.5/33mer gliadin peptide-coated beads and incubated for 60 minutes with shaking at 4 degrees C. and washed with blocking buffer. Final concentration of S2 TCR tetramer-PE was 2.0 micro g/mL. Data acquisition was performed on a LSR Fortessa (Becton Dickinson), followed by analysis using FlowJo software (Tree Star) and GraphPad Prism software (GraphPad).

As shown in FIG. 13, the antibodies have neutralizing activity against the binding between gliadin-bound HLA-DQ2.5 and S2 TCR. Thus, it was indicated that the antibodies of the present invention can block the interaction between HLA-DQ2.5 and an HLA-DQ2.5-restricted CD4+ T cell.

4.5. Binding Analysis of the Antibodies to HLA-DQ2.5/Invariant Chain

The binding response of anti-HLA-DQ antibodies to the HLA-DQ2.5/invariant chain complex at pH 7.4 was determined at 25 degrees C. using Biacore 8K instrument (GE Healthcare). Anti-human Fc (GE Healthcare) was immobilized onto all flow cells of a CM4 sensor chip using amine coupling kit (GE Healthcare). All antibodies and analytes were prepared in ACES pH 7.4 containing 20 mM ACES, 150 mM NaCl, 0.05% Tween 20, and 0.005% NaN3. Each antibody was captured onto the sensor surface by anti-human Fc. Antibody capture levels were aimed at 200 resonance unit (RU). The recombinant HLA-DQ2.5/invariant chain complex was injected at 100 nM, followed by dissociation. Sensor surface was regenerated after each cycle with 3M MgCl2.

The binding level of the anti-HLA-DQ2.5 antibodies towards HLA-DQ2.5/invariant chain was monitored from the binding response. The binding level was normalized to the capture level of the corresponding anti-HLA-DQ2.5 antibody.

Since the amount of the antibody captured on the tip varied, the ratio of the binding level to the capture level was used for evaluation of the binding activity. As shown in FIG. 14, no significant binding towards HLA-DQ2.5/invariant chain was observed for the anti-HLA-DQ2.5 antibodies. Thus, it is thought that the antibodies of the present invention do not specifically bind to the complex of the invariant chain and HLA-DQ.

As mentioned above, when HLA-DQ forms a complex with the invariant chain, the complex on the cell surface is rapidly internalized into the endosome (“rapid internalization”, where T_{1/2} is around 3.2 min). After degradation of the invariant chain in the endosome, the HLA-DQ/peptide complex is transferred to the cell surface, and then recognized by TCR on T cells. The complex without the invariant chain is slowly internalized into the endosome (“slow internalization”, where T_{1/2} is 789-1500 min).

The fact that the antibodies of the present invention do not significantly bind to HLA-DQ in the presence of the invariant chain indicates that the antibodies are less susceptible to rapid internalization which can cause the antibodies to be quickly transferred to the endosome and degraded. The absence of rapid cell internalization (i.e., rapid endosomal degradation) of the antibodies of the present invention is thought to be useful.

4.6. Cell-Based Neutralizing Assay

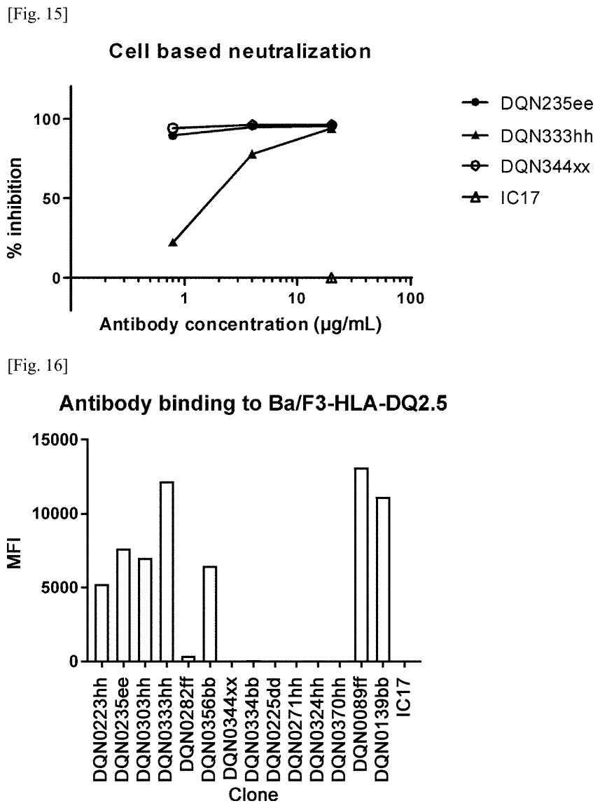

Cell-based neutralizing activity was confirmed Ba/F3 cells expressing the HLA-DQ2.5/33mer gliadin peptide complex (Ba/F3-HLA-DQ2.5/33mer gliadin peptide) were distributed in 96 well plates (Corning, 3799). Serially-diluted anti-HLA-DQ antibodies and D2 TCR-expressing J.RT-T3.5 cells were then added and cultured at 37 degrees C., 5% CO2 for overnight. Final concentration of Ba/F3-HLA-DQ2.5/33mer gliadin peptide was 3.0×10⁴ cells/well, D2 TCR-expressing J.RT-T3.5 cells was 1.0×10⁵ cells/well, and final assay volume was 100 micro L/well. After overnight culture, cells were harvested and washed by FACS buffer (2% FBS, 2 mM EDTA in PBS). 30 times diluted APC anti-mouse CD45 antibody (Biolegend, 103112), and 40 times diluted Brilliant Violet 421™ anti-human CD69 Antibody (Biolegend, 410930) were then added and incubated for 30 minutes at 4 degrees C., and washed and re-suspended with FACS buffer. Data acquisition was performed on a LSR Fortessa (Becton Dickinson), followed by analysis using FlowJo software (Tree Star) and GraphPad Prism software (GraphPad) to determine neutralizing activity of anti-HLA-DQ antibodies on the activation of J.RT-T3.5 cells. CD69 expression on J.RT-T3.5 cells was used for an activation marker. As shown in FIG. 15, the antibodies inhibit the activation of D2 TCR-expressing T cells induced by Ba/F3 cells expressing the DQ2.5/33mer gliadin peptide complex.

### Example 5

Characteristics of the Anti-HLA-DQ2.5 Antibodies

Table 6 shows alignments of the alpha and beta chains of major HLA-DQ isoforms. The alpha 1 domain and beta 1 domain, which together form a loading site for a peptide (e.g. gluten peptide), are indicated in the table. The alpha 1 domain is located at positions 24-109 of the alpha chain, and the beta 1 domain is located at positions 33-127 of the beta chain (information from Mucosal Immunol. 2011 January; 4(1): 112-120). It is thought that the TCR-binding site overlaps or is located around the above positions. These positions are also expected to contain at least part of the epitope of anti-HLA-DQ2.5 antibodies which block the binding between HLA-DQ2.5 and TCR.

The sequence ID numbers for the HLA-DQ chain sequences shown in Table 6 are as follows. The sequences of the alpha (“A” in Table 6) chains of HLA-DQ2.5, 2.2, 5.1, 6.1, 6.3, 6.4, 7.3, 7.5, and 8 are respectively shown in SEQ ID NOs: 112 to 120. The sequences of the beta (“B” in Table 6) chains of HLA-DQ2.5, 2.2, 5.1, 6.1, 6.3, 6.4, 7.3, 7.5, and 8 are respectively shown in SEQ ID NOs: 121 to 129.

TABLE 6
Alignment of HLA-DQ alpha chain α1 domain

| | | | |
|---|---|---|---|
| DQ2.5(A) DQ2.2(A) DQ5.1(A) DQ6.1(A) DQ6.3(A) DQ6.4(A) DQ7.3(A) DQ7.5(A) DQ8(A) | 1 1 1 1 1 1 1 1 1 | 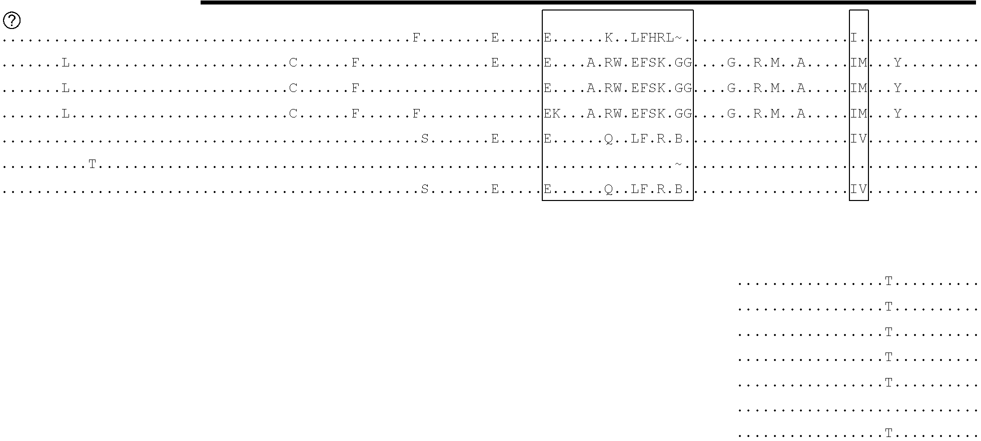 | 139 139 140 140 140 140 140 139 140 |
| DQ2.5(A) | 140 | [symbol] | 254 |
| DQ2.2(A) | 140 | ......................................F....D.I...........E.......................................I................. | 254 |
| DQ5.1(A) | 141 | ...........Q..........................F....D.I...........Q..........................................Q.............. | 255 |
| DQ6.1(A) | 141 | ...........Q..........................F....D.I...........Q...............................M..........Q.............. | 255 |
| DQ6.3(A) | 141 | ............A.........................F....D.I...........Q..........................................Q.............. | 255 |
| DQ6.4(A) | 141 | ...........Q..........................F....D.I...........Q...............................M..........Q.............. | 255 |
| DQ7.3(A) | 141 | ......................................F...DD.I...........E...........T............................L................ | 255 |
| DQ7.5(A) | 140 | ................................................................................................................... | 254 |
| DQ8(A) | 141 | ......................................F....D.I...........E...........T............................L................ | 255 |

Alignment of HLA-DQ beta chain β1 domain

| | | | |
|---|---|---|---|
| DQ2.5(B) DQ2.2(B) DQ5.1(B) DQ6.1(B) DQ6.3(B) DQ6.4(B) DQ7.3(B) DQ7.5(B) DQ8(B) | 1 1 1 1 1 1 1 1 1 | 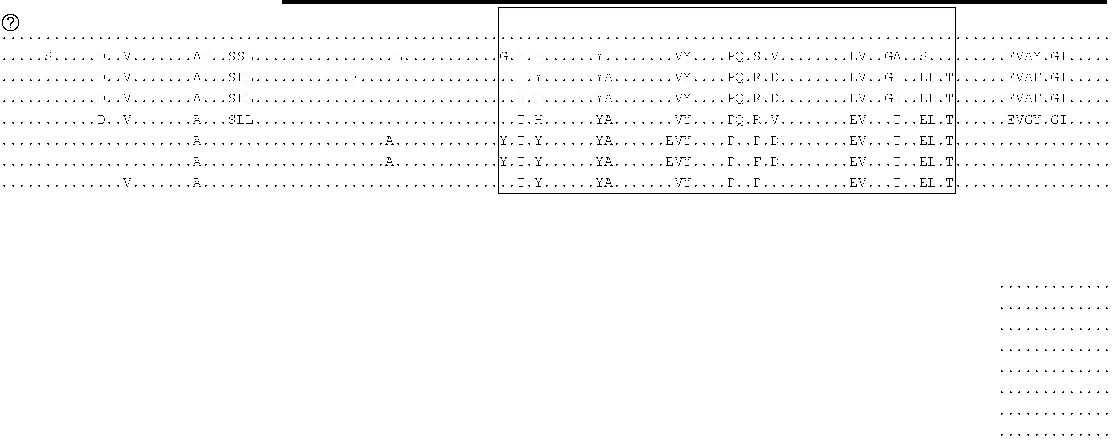 | 140 140 140 140 140 140 140 140 140 |
| DQ2.5(B) | 141 | [symbol] | 261 |
| DQ2.2(B) | 141 | ..........................G..............................................................................................~~~~~~~~ | 261 |
| DQ5.1(B) | 141 | .......I........S.............................................................................V................RQ..R.....~~~~~~~~ | 261 |
| DQ6.1(B) | 141 | ................G.............................................................................V................RQ........~~~~~~~~ | 261 |
| DQ6.3(B) | 141 | ................G.............................................................................V................RQ........~~~~~~~~ | 261 |
| DQ6.4(B) | 141 | ................G....Q........................................................................V................RQ........~~~~~~~~ | 261 |
| DQ7.3(B) | 141 | ...............................T..........................H..............N...............................................~~~~~~~~ | 261 |
| DQ7.5(B) | 141 | ...............................T..........................H..............N...............................................~~~~~~~~ | 261 |
| DQ8(B) | 141 | ...............................T.........................................N..T............................................~~~~~~~~ | 261 |

[symbol]

indicates data missing or illegible when filed

The results of Example 4 show that DQN0223hh, DQN0235ee, and DQN0303hh have binding activity to both HLA-DQ2.5 and HLA-DQ2.2, and substantially no binding activity to the other HLA-DQ isoforms. The similarity of the beta chain between HLA-DQ2.5 and HLA-DQ2.2 suggests that these antibodies have binding activity to the beta chain of HLA-DQ2.5. The epitope of these antibodies possibly comprises at least a part of amino acids 58 to 109 of the beta chain.

DQN0333hh and DQN0356bb have binding activity to both HLA-DQ2.5 and HLA-DQ7.5 and substantially no binding activity to the other HLA-DQ isoforms. The similarity of the alpha chain between HLA-DQ2.5 and HLA-DQ7.5 suggests that these antibodies have binding activity to the alpha chain of HLA-DQ2.5. The epitope of these antibodies possibly comprises at least a part of amino acids 63 to 78 and/or 97 to 98 of the alpha chain.

DQN0344xx and DQN0334bb have binding activity to HLA-DQ2.5 only and substantially no binding activity to the other HLA-DQ isoforms. It is expected that these antibodies have binding activity to both alpha chain and beta chain of HLA-DQ2.5. The epitope of these antibodies possibly comprises at least a part of both amino acids 58 to 109 of the beta chain and amino acids 63 to 78 and/or 97 to 98 of the alpha chain.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

### Example 6

Establishment of Ba/F3 Cell Lines Expressing HLA-DQ2.5/alpha 1 gliadin peptide, HLA-DQ2.5/alpha 1b gliadin peptide, HLA-DQ2.5/alpha 2 gliadin peptide, HLA-DQ2.5/omega 1 gliadin peptide, HLA-DQ2.5/omega 2 gliadin peptide, HLA-DQ2.5/secalin 1 peptide, HLA-DQ2.5/secalin 2 peptide, HLA-DQ2.5/salmonella peptide, HLA-DQ2.5/Mycobacterium bovis peptide, HLA-DQ2.5/Hepatitis B virus peptide.

HLA-DQA1*0501 cDNA (IMGT/HLA accession No. HLA00613) was inserted into the expression vector pCXND3 (WO2008/156083).

HLA-DQB1*0201 cDNA (IMGT/HLA accession No. HLA00622) was inserted into the expression vector pCXZD1 (US/20090324589). HLA-DQB1*0201 for the HLA-DQ2.5/alpha 1 gliadin peptide complex has alpha 1 gliadin peptide sequence: QPFPQPELPYPGS (SEQ ID NO: 130), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/alpha 1b gliadin peptide complex has alpha 1b gliadin peptide sequence: QLPYPQPELPYPGS (SEQ ID NO: 131), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/alpha 2 gliadin peptide complex has alpha 2 gliadin peptide sequence: APQPELPYPQPGS (SEQ ID NO: 132), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/omega 1 gliadin peptide complex has omega 1 gliadin peptide sequence: QQPFPQPEQPFPGS (SEQ ID NO: 133), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/omega 2 gliadin peptide complex has omega 2 gliadin peptide sequence: QFPQPEQPFPWQGS (SEQ ID NO: 134), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/secalin 1 peptide complex has secalin 1 peptide sequence: QPEQPFPQPEQPFPQGS (SEQ ID NO: 135), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/secalin 2 peptide complex has secalin 2 peptide sequence: QQPFPQPEQPFPQSQGS (SEQ ID NO: 136), and factor X cleavage linker: (Acta Crystallogr Sect F Struct Biol Cryst Commun. 2007 Dec. 1; 63 (Pt 12): 1021-1025.) on the N-terminus of HLA-DQB1*0201. HLA-DQB1*0201 for the HLA-DQ2.5/salmonella peptide complex has salmonella peptide sequence: MMAWRMMRY (SEQ ID NO: 137), and GSGGGS linker on the N-terminus of HLA-DQB1*0201. HLA-DQB1*0201 for the HLA-DQ2.5/Mycobacterium bovis peptide complex has Mycobacterium bovis peptide sequence: KPLLIIAEDVEGEY (SEQ ID NO: 138), and GSGGGS linker on the N-terminus of HLA-DQB1*0201. HLA-DQB1*0201 for the HLA-DQ2.5/Hepatitis B virus peptide complex has Hepatitis B virus peptide sequence: PDRVHFASPLHVAWR (SEQ ID NO: 139), and GSGGGS linker on the N-terminus of HLA-DQB1*0201.

Each of the linearized HLA-DQA1*0501-pCXND3 and HLA-DQB1*0201 for HLA-DQ2.5/alpha 1 gliadin peptide -pCXZD1, HLA-DQA1*0501-pCXND3 and HLA-DQB1*0201 for HLA-DQ2.5/alpha 1b gliadin peptide -pCXZD1, HLA-DQA1*0501-pCXND3 and HLA-DQB1*0201 for HLA-DQ2.5/alpha 2 gliadin peptide -pCXZD1, HLA-DQA1*0501-pCXND3 and HLA-DQB1*0201 for HLA-DQ2.5/omega 1 gliadin peptide -pCXZD1, HLA-DQA1*0501-pCXND3 and HLA-DQB1*0201 for HLA-DQ2.5/omega 2 gliadin peptide -pCXZD1, HLA-DQA1*0501-pCXND3 and HLA-DQB1*0201 for HLA-DQ2.5/secalin 1 peptide -pCXZD1, HLA-DQA1*0501-pCXND3 and HLA-DQB1*0201 for HLA-DQ2.5/secalin 2 peptide -pCXZD1, HLA-DQA1*0501-pCXND3 and HLA-DQB1*0201 for HLA-DQ2.5/salmonella peptide -pCXZD1, HLA-DQA1*0501-pCXND3 and HLA-DQB1*0201 for HLA-DQ2.5/Mycobacterium bovis peptide -pCXZD1, HLA-DQA1*0501-pCXND3 and HLA-DQB1*0201 for HLA-DQ2.5/Hepatitis B virus peptide -pCXZD1 were simultaneously introduced into mouse IL-3-dependent pro-B cell-derived cell line Ba/F3 by electroporation (LONZA, 4D-Nucleofector X). Transfected cells were then cultured in media containing Geneticin and Zeocin. Cultured and expanded cell was then checked the expression of HLA-DQ2.5 molecule and confirmed high expression of HLA-DQ2.5. Established each cell lines were named HLA-DQ2.5/33mer gliadin peptide (HLA-DQA1*0501, HLA-DQB1*0201 for HLA-DQ2.5/33mer gliadin peptide), HLA-DQ2.5/alpha 1 gliadin peptide (HLA-DQA1*0501, HLA-DQB1*0201 for HLA-DQ2.5/alpha 1 gliadin peptide), HLA-DQ2.5/alpha 1b gliadin peptide (HLA-DQA1*0501, HLA-DQB1*0201 for HLA-DQ2.5/alpha 1b gliadin peptide), HLA-DQ2.5/alpha 2 gliadin peptide (HLA-DQA1*0501, HLA-DQB1*0201 for HLA-DQ2.5/alpha 2 gliadin peptide), HLA-DQ2.5/omega 1 gliadin peptide (HLA-DQA1*0501, HLA-DQB1*0201 for HLA-DQ2.5/omega 1 gliadin peptide), HLA-DQ2.5/omega 2 gliadin peptide (HLA-DQA1*0501, HLA-DQB1*0201 for HLA-DQ2.5/omega 2 gliadin gliadin peptide), HLA-DQ2.5/secalin 1 peptide (HLA-DQA1*0501, HLA-DQB1*0201 for HLA-DQ2.5/secalin 1 peptide), HLA-DQ2.5/secalin 2 peptide (HLA-DQA1*0501, HLA-DQB1*0201 for HLA-DQ2.5/secalin 2 peptide), HLA-DQ2.5/salmonella peptide (HLA-DQA1*0501, HLA-DQB1*0201 for HLA-DQ2.5/salmonella peptide), HLA-DQ2.5/Mycobacterium bovis peptide (HLA-DQA1*0501, HLA-DQB1*0201 for HLA-DQ2.5/Mycobacterium bovis peptide), HLA-DQ2.5/Hepatitis B virus peptide (HLA-DQA1*0501, HLA-DQB1*0201 for HLA-DQ2.5/Hepatitis B virus peptide).

### Example 7

Binding Analysis of the Antibodies to HLA-DQ2.5

FIGS. 16 to 28 show the binding of the anti-HLA-DQ antibodies to a panel of HLA-DQ in the form of a complex with several peptides-expressing Ba/F3 cell lines as determined by FACS. The binding of anti-HLA-DQ antibodies to Ba/F3-HLA-DQ2.5 (expressing HLA-DQ2.5), Ba/F3-HLA-DQ2.5/33mer gliadin peptide (expressing HLA-DQ2.5/33mer gliadin peptide), Ba/F3-HLA-DQ2.5/CLIP peptide (expressing HLA-DQ2.5/CLIP peptide), Ba/F3-HLA-DQ2.5/alpha 1 gliadin peptide (expressing HLA-DQ2.5/alpha 1 gliadin peptide), Ba/F3-HLA-DQ2.5/alpha 1b gliadin peptide (expressing HLA-DQ2.5/alpha 1b gliadin peptide), Ba/F3-HLA-DQ2.5/alpha 2 gliadin peptide (expressing HLA-DQ2.5/alpha 2 gliadin peptide), Ba/F3-HLA-DQ2.5/omega 1 gliadin peptide (expressing HLA-DQ2.5/omega 1 gliadin peptide), Ba/F3-HLA-DQ2.5/omega 2 gliadin peptide (expressing HLA-DQ2.5/omega 2 gliadin peptide), Ba/F3-HLA-DQ2.5/secalin 1 peptide (expressing HLA-DQ2.5/secalin 1 peptide), Ba/F3-HLA-DQ2.5/secalin 2 peptide (expressing HLA-DQ2.5/secalin 2 peptide), Ba/F3-HLA-DQ2.5/salmonella peptide (expressing HLA-DQ2.5/salmonella peptide), Ba/F3-HLA-DQ2.5/Mycobacterium bovis peptide (expressing HLA-DQ2.5/Mycobacterium bovis peptide), Ba/F3-HLA-DQ2.5/Hepatitis B virus peptide (expressing HLA-DQ2.5/Hepatitis B virus peptide) was tested. 10 microgram/mL of anti-HLA-DQ antibodies were incubated with each cell line for 30 minutes at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 20 minutes at 4 degrees C., and this was washed with FACS buffer. Data acquisition was performed on LSRFortessa X-20 (Becton Dickinson), followed by analysis using the FlowJo software (Tree Star) and GraphPad Prism software (GraphPad).

FIG. 16 shows that DQN0223hh, DQN0235ee, DQN0303hh, DQN0333hh, DQN0356bb, DQN0089ff, and DQN0139bb have binding activity to HLA-DQ2.5 which is not in the form of a complex with gliadin peptide or secalin peptide, CLIP peptide, salmonella peptide, Mycobacterium bovis peptide, Hepatitis B virus peptide whereas DQN0282ff, DQN0344xx, and DQN0334bb, DQN0225dd, DQN0271hh, DQN0324hh, DQN0370hh have substantially no binding activity to the same.

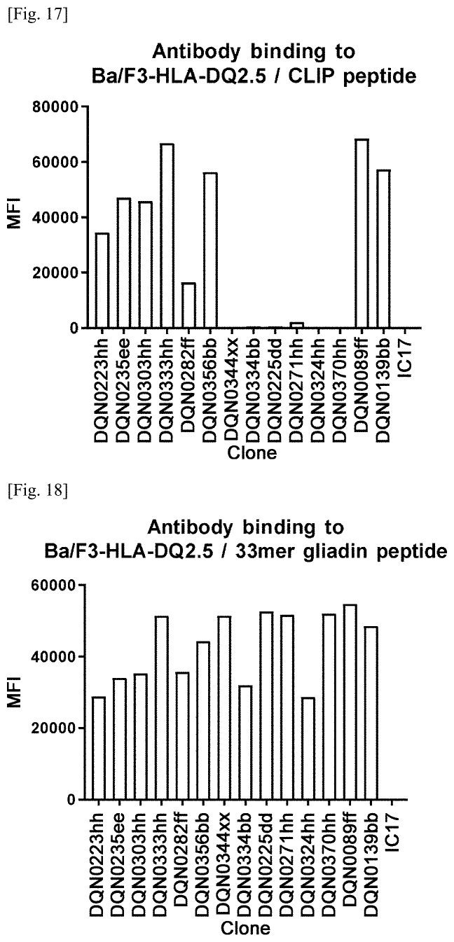

FIG. 17 shows that DQN0223hh, DQN0235ee, DQN0303hh, DQN0333hh, DQN0282ff, DQN0356bb, DQN0089ff, and DQN0139bb have binding activity to the HLA-DQ2.5/CLIP peptide complex, whereas DQN0344xx, DQN0334bb, DQN0225dd, DQN0271hh, DQN0324hh, DQN0370hh have substantially no binding activity to the same. IC17 shows the level of a negative control (background) in which the anti-HLA-DQ2.5 antibody was not added in the experiments. The same applies to the other Figures.

FIGS. 18-25 show that DQN0223hh, DQN0235ee, DQN0303hh, DQN0333hh, DQN0282ff, DQN0356bb, DQN0225dd, DQN0271hh, DQN0324hh, DQN0370hh, DQN0089ff, and DQN0139bb have binding activity to the HLA-DQ2.5 in the form of a complex with 33mer gliadin peptide, alpha 1 gliadin peptide, alpha 1b gliadin peptide, alpha 2 gliadin peptide, omega 1 gliadin peptide, omega 2 gliadin peptide, secalin 1 peptide and secalin 2 peptide. DQN0344xx has binding activity to the HLA-DQ2.5 in the form of a complex with 33mer gliadin peptide, alpha 1 gliadin peptide, alpha 1b gliadin peptide, alpha 2 gliadin peptide, omega 1 gliadin peptide, secalin 1 peptide and secalin 2 peptide. DQN0334bb has binding activity to the HLA-DQ2.5 in the form of a complex with 33mer gliadin peptide, alpha 1 gliadin peptide, alpha 1b gliadin peptide, alpha 2 gliadin peptide, omega 2 gliadin peptide and secalin 1 peptide.

FIGS. 26-28 show that DQN0223hh, DQN0235ee, DQN0303hh, DQN0333hh, DQN0356bb, DQN0089ff, and DQN0139bb have binding activity to HLA-DQ2.5 in the form of a complex with salmonella peptide, Mycobacterium bovis peptide, and Hepatitis B virus peptide which is not gluten derived peptides, whereas DQN0282ff, DQN0344xx, and DQN0334bb, DQN0225dd, DQN0271hh, DQN0324hh, DQN0370hh have substantially no binding activity to the same.

### Example 8

Binding Analysis of the Antibodies to HLA-DQ2.5 Positive PBMC-B Cell

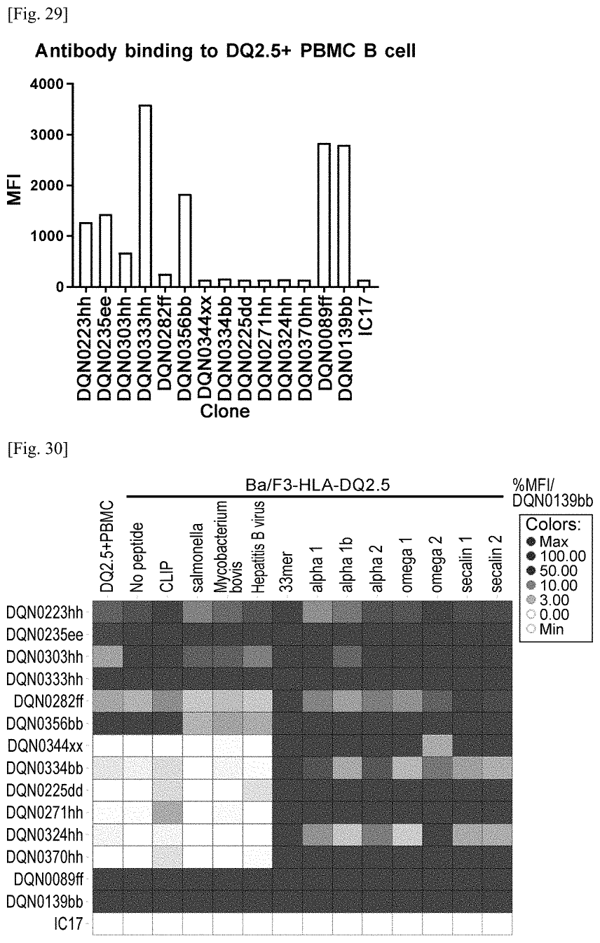

FIG. 29 shows the binding of the anti-HLA-DQ antibodies to HLA-DQ2.5 positive PBMC-B cell as determined by FACS. 10 microgram/mL of anti-HLA-DQ antibodies were incubated with PBMC in the presence of human FcR blocking reagent (Miltenyi Biotech, Cat. 130-059-901) for 30 minutes at room temperature and washed with FACS buffer (2% FBS, 2 mM EDTA in PBS). Pacific Blue™ anti-human CD19 Antibody mouse IgG1k (Biolegend, Cat. 2043-09) and Alexa Fluor 555 labeled anti-human IgG Fc antibody (reference example 1 to 3) was then added and incubated for 30 minutes at 4 degrees C., and this was washed with FACS buffer. Data acquisition was performed on LSRFortessa X-20 (Becton Dickinson), followed by analysis using the FlowJo software (Tree Star) and GraphPad Prism software (GraphPad).

FIG. 29 shows that DQN0223hh, DQN0235ee, DQN0303hh, DQN0333hh, DQN0356bb, DQN0089ff, and DQN0139bb have binding activity to HLA-DQ2.5 positive PBMC-B cell whereas DQN0282ff, DQN0344xx, and DQN0334bb, DQN0225dd, DQN0271hh, DQN0324hh, DQN0370hh have substantially no binding activity to the same. FIG. 30 is the summary of FIGS. 16-29. DQN0223hh, DQN0235ee, DQN0303hh, DQN0333hh, DQN0356bb, DQN0089ff, and DQN0139bb have binding activity to HLA-DQ2.5 in the form of a complex with or without any peptide, whereas DQN0344xx, DQN0334bb, DQN0225dd, DQN0271hh, DQN0324hh, DQN0370hh have binding activity to HLA-DQ2.5 only when in the complex with gluten derived peptides, in particular gluten 33mer gliadin peptide, alpha 1 gliadin peptide, alpha 1b gliadin peptide, alpha 2 gliadin peptide, omega 1 gliadin peptide, omega 2 gliadin peptide, secalin 1 peptide and secalin 2 peptide with a few exceptions. On the other hand, DQN0344xx, DQN0334bb, DQN0225dd, DQN0271hh, DQN0324hh, DQN0370hh have substantially no binding activity to HLA-DQ2.5 without peptide, and HLA-DQ2.5 in the form of a complex with peptides which are irrelevant to gluten derived peptide. Numeral data for FIG. 30 are shown in Table 7.

TABLE 7

| | DQ2.5 + | No | | | Mycobacterium | Hepatitis | |
|---|---|---|---|---|---|---|---|
| | PBMC | peptide | CLIP | salmonella | bovis | B virus | 33mer |
| DQN0223hh | 42.8 | 46.9 | 60.2 | 32.9 | 40.2 | 46.1 | 59.5 |
| DQN0235ee | 48.6 | 68.5 | 82.0 | 60.4 | 68.9 | 66.9 | 70.1 |
| DQN0303hh | 20.0 | 62.7 | 80.0 | 42.9 | 43.0 | 33.8 | 72.8 |
| DQN0333hh | 129.8 | 109.2 | 116.5 | 109.0 | 111.7 | 109.2 | 105.9 |
| DQN0282ff | 4.3 | 3.0 | 28.7 | 2.0 | 2.4 | 1.9 | 73.5 |
| DQN0356bb | 63.6 | 58.0 | 98.3 | 11.5 | 20.3 | 15.2 | 91.1 |
| DQN0344xx | −0.1 | 0.0 | 0.0 | −0.1 | 0.0 | −0.1 | 105.9 |
| DQN0334bb | 0.8 | 0.4 | 1.2 | 0.0 | 0.3 | 0.1 | 65.9 |
| DQN0225dd | −0.1 | −0.1 | 1.2 | −0.2 | 0.0 | 1.0 | 108.3 |
| DQN0271hh | 0.0 | 0.0 | 3.8 | −0.1 | 0.1 | −0.1 | 106.3 |
| DQN0324hh | 0.3 | 0.0 | 0.3 | −0.1 | −0.1 | −0.1 | 59.2 |
| DQN0370hh | −0.1 | 0.0 | 1.0 | −0.1 | 0.0 | 0.0 | 107.1 |
| DQN0089ff | 101.4 | 117.6 | 119.4 | 115.7 | 112.5 | 116.5 | 112.7 |
| DQN0139bb | 100.0 | 100.0 | 100.0 | 100.0 | 100.0 | 100.0 | 100.0 |
| IC17 | 0.0 | 0.0 | 0.0 | 0.0 | 0.0 | 0.0 | 0.0 |

| | alpha | alpha | alpha | omega | omega | secalin | secalin |
|---|---|---|---|---|---|---|---|
| | 1 | 1b | 2 | 1 | 2 | 1 | 2 |
| DQN0223hh | 27.9 | 36.0 | 46.8 | 46.0 | 59.0 | 47.6 | 48.4 |
| DQN0235ee | 52.3 | 66.9 | 81.3 | 75.2 | 78.8 | 73.4 | 71.1 |
| DQN0303hh | 51.4 | 41.5 | 62.6 | 56.2 | 91.7 | 64.5 | 59.4 |
| DQN0333hh | 109.2 | 107.7 | 117.6 | 106.0 | 128.3 | 109.5 | 106.4 |
| DQN0282ff | 32.6 | 20.9 | 34.5 | 7.0 | 42.6 | 49.6 | 48.0 |
| DQN0356bb | 94.9 | 88.0 | 85.6 | 76.0 | 68.8 | 74.5 | 77.8 |
| DQN0344xx | 89.7 | 75.3 | 81.9 | 68.8 | 3.7 | 73.9 | 60.6 |
| DQN0334bb | 46.4 | 16.5 | 45.3 | 2.8 | 36.6 | 5.0 | 3.3 |
| DQN0225dd | 93.5 | 81.3 | 85.1 | 75.6 | 62.0 | 77.2 | 68.7 |
| DQN0271hh | 91.2 | 80.5 | 83.0 | 79.3 | 68.9 | 65.3 | 70.2 |
| DQN0324hh | 26.9 | 8.2 | 34.9 | 7.3 | 79.4 | 16.3 | 14.5 |
| DQN0370hh | 91.2 | 79.2 | 83.7 | 73.7 | 65.8 | 79.2 | 68.1 |
| DQN0089ff | 107.0 | 102.0 | 106.8 | 101.0 | 106.7 | 101.3 | 103.3 |
| DQN0139bb | 100.0 | 100.0 | 100.0 | 100.0 | 100.0 | 100.0 | 100.0 |
| IC17 | 0.0 | 0.0 | 0.0 | 0.0 | 0.0 | 0.0 | 0.0 |

### Example 9

Binding Analysis of the Antibodies to HLA-DQ2.2 and HLA-DQ7.5

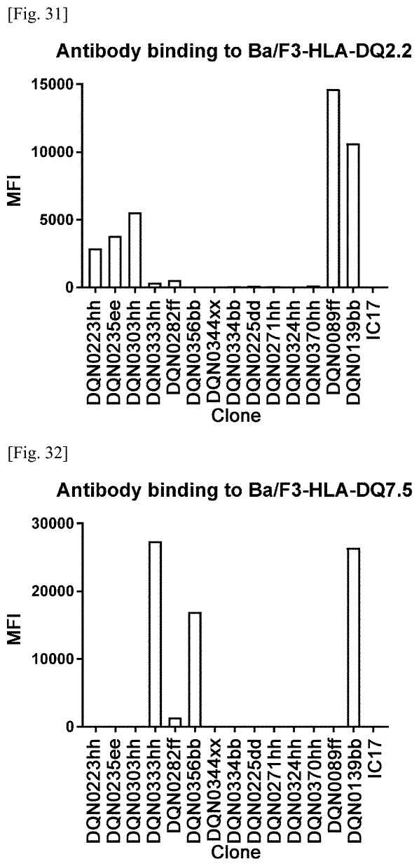

FIGS. 31 and 32 show the binding of the anti-HLA-DQ antibodies to a panel of multiple MHC class II-expressing Ba/F3 cell lines as determined by FACS. The binding of anti-HLA-DQ antibodies to Ba/F3-HLA-DQ2.2 (expressing HLA-DQ2.2), Ba/F3-HLA-DQ7.5 (expressing HLA-DQ7.5) was tested. 10 microgram/mL of anti-HLA-DQ antibodies were incubated with each cell line for 30 minutes at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 20 minutes at 4 degrees C., and this was washed with FACS buffer. Data acquisition was performed on LSRFortessa X-20 (Becton Dickinson), followed by analysis using the FlowJo software (Tree Star) and GraphPad Prism software (GraphPad).

FIG. 31 shows that DQN0223hh, DQN0235ee, DQN0303hh, DQN0089ff, and DQN0139bb have binding activity to HLA-DQ2.2, whereas DQN0333hh, DQN0282ff, DQN0356bb, DQN0344xx, and DQN0334bb, DQN0225dd, DQN0271hh, DQN0324hh, DQN0370hh have substantially no binding activity to the same.

FIG. 32 shows that DQN0333hh, DQN0356bb, and DQN0139bb have binding activity to HLA-DQ7.5, whereas DQN0223hh, DQN0235ee, DQN0303hh, DQN0282ff, DQN0344xx, DQN0334bb, DQN0225dd, DQN0271hh, DQN0324hh, DQN0370hh and DQN0089ff have substantially no binding activity to the same.

### Example 10

Binding Analysis of the Antibodies to HLA-DQ8/5.1/6.3/7.3 and HLA-DR/DP

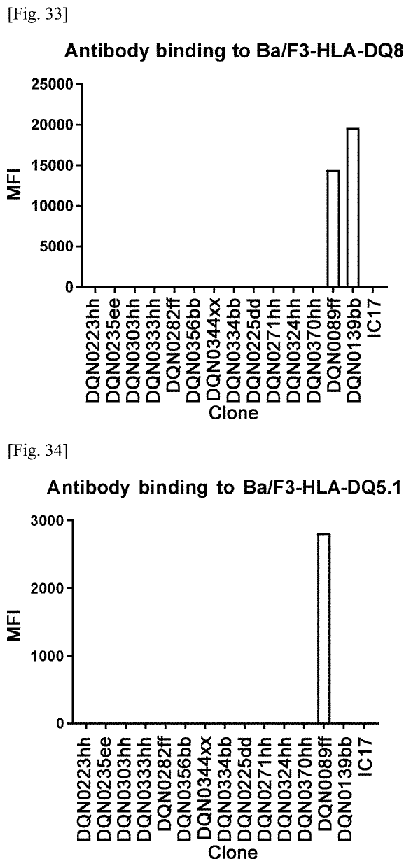

FIGS. 33 to 38 show the binding of the anti-HLA-DQ antibodies to multiple MHC class II-expressing Ba/F3 cell lines as determined by FACS. The binding of the anti-HLA-DQ antibodies to Ba/F3-HLA-DQ8, BaF3-HLA-DQ5.1, BaF3-HLA-DQ6.3, Ba/F3-HLA-DQ7.3, and Ba/F3-HLA-DR, Ba/F3-HLA-DP was tested. 20 microgram/mL of anti-HLA-DQ antibodies were incubated with each cell line for 30 minutes at room temperature and washed with FACS buffer (2% FBS, 2 mM EDTA in PBS). Goat F(ab′)2 anti-Human IgG, Mouse ads-PE (Southern Biotech, Cat. 2043-09) was then added and incubated for 20 minutes at 4 degrees C. and washed with FACS buffer. Data acquisition was performed on LSRFortessa X-20 (Becton Dickinson), followed by analysis using the FlowJo software (Tree Star) and GraphPad Prism software (GraphPad).

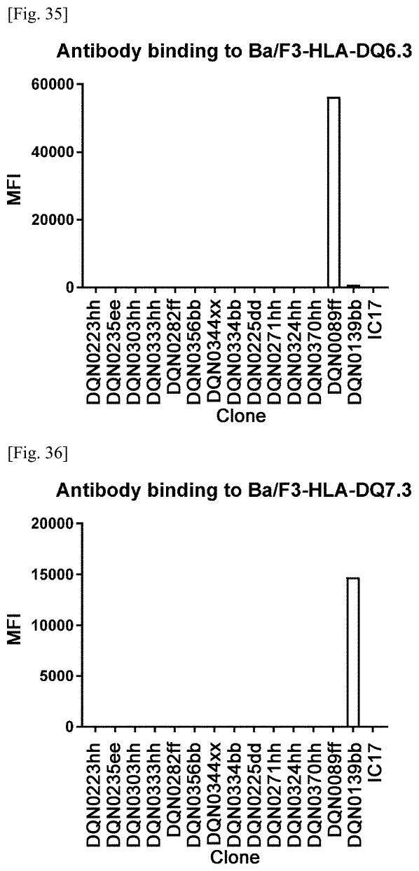

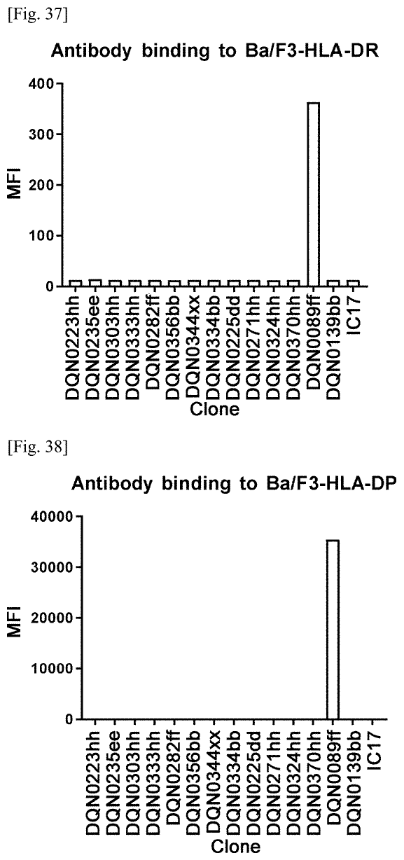

FIGS. 37 and 38 show that DQN0089ff has binding activity to HLA-DP/DR, whereas the other antibodies have substantially no binding activity to the same. FIG. 33 shows that DQN0089ff and DQN0139bb have binding activity to HLA-DQ8, whereas the other antibodies have substantially no binding activity to the same. FIGS. 34 and 35 show that DQN0089ff has binding activity to HLA-DQ5.1/6.3, whereas the other antibodies have substantially no binding activity to the same. FIG. 36 shows that DQN0139bb has binding activity to HLA-DQ7.3, whereas the other antibodies have substantially no binding activity to the same.

### Example 11

Cell-Based Neutralizing Assay

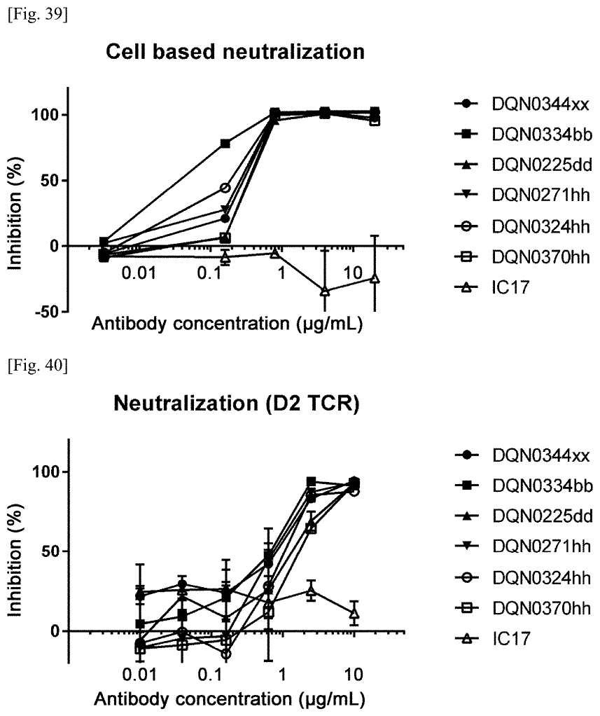

Cell-based neutralizing activity was confirmed Ba/F3 cells expressing the HLA-DQ2.5/33mer gliadin peptide complex (Ba/F3-HLA-DQ2.5/33mer gliadin peptide) were distributed in 96 well plates (Corning, 3799). Serially-diluted anti-HLA-DQ antibodies and D2 TCR-expressing J.RT-T3.5 cells were then added and cultured at 37 degrees C., 5% CO2 for overnight. Final concentration of Ba/F3-HLA-DQ2.5/33mer gliadin peptide was 3.0×10⁴ cells/well, D2 TCR-expressing J.RT-T3.5 cells was 1.0×10⁵ cells/well, and final assay volume was 100 micro L/well. After overnight culture, cells were harvested and washed by FACS buffer (2% FBS, 2 mM EDTA in PBS). 30 times diluted APC anti-mouse CD45 antibody (Biolegend, 103112), and 40 times diluted Brilliant Violet 421™ anti-human CD69 Antibody (Biolegend, 410930) were then added and incubated for 30 minutes at 4 degrees C., and washed and re-suspended with FACS buffer. Data acquisition was performed on a LSR Fortessa (Becton Dickinson), followed by analysis using FlowJo software (Tree Star) and GraphPad Prism software (GraphPad) to determine neutralizing activity of anti-HLA-DQ antibodies on the activation of J.RT-T3.5 cells. CD69 expression on J.RT-T3.5 cells was used for an activation marker. As shown in FIG. 39, the antibodies inhibit the activation of D2 TCR-expressing T cells induced by Ba/F3 cells expressing the DQ2.5/33mer gliadin peptide complex.

### Example 12

AlphaLISA Neutralizing Assay (HLA-DQ2.5/33Mer Gliadin Peptide—D2 TCR)

The neutralizing activity of anti-HLA-DQ antibodies towards the binding of the HLA-DQ2.5/33mer gliadin peptide complex and D2 TCR was assessed using the AlphaLISA beads assay platform. 40 micro g/mL of Streptavidin-AlphaLISA acceptor beads (Perkin Elmer, AL125M) were immobilized with 10 nM of biotinylated HLA-DQ2.5/33mer gliadin peptide in alphascreen buffer at pH 7.4 (40 mM HEPES/NaOH (pH 7.4), 100 mM NaCl, 1 mM CaCl2, 0.1% BSA, 0.05% Tween-20) for 60 minutes at room temperature. Simultaneously, 80 micro g/mL of Streptavidin-Alphascreen donor beads (Perkin Elmer, 6760002) were immobilized with 2.5 nM of biotinylated D2 TCR in alphascreen buffer for 60 minutes at room temperature. Then, using a 384-well plate, 10 micro L of serially diluted anti-HLA-DQ antibodies were incubated with 5 micro L of the HLA-DQ2.5/33mer gliadin peptide-coated acceptor beads and 5 micro L of the D2 TCR-coated donor beads for 60 minutes at room temperature. Alphascreen signal (counts per second, CPS) was measured by SpectraMax Paradigm (Molecular Devices), followed by analysis using GraphPad Prism software (GraphPad).

As shown in FIG. 40, the antibodies have neutralizing activity against the binding between gliadin-bound HLA-DQ2.5 and D2 TCR.

### Example 13

Beads Neutralizing Assay (HLA-DQ2.5/33Mer Gliadin Peptide—S2 TCR)

The neutralizing activity of the anti-HLA-DQ antibodies towards the binding of the HLA-DQ2.5/33mer gliadin peptide complex and S2 TCR was assessed using the Beads assay platform. Streptavidin-coated yellow particles (Spherotech, SVFB-2552-6K) were incubated in blocking buffer (2% BSA in PBS) for 30 minutes with shaking at room temperature. After centrifuging and aspirating supernatant, the soluble HLA-DQ2.5/33mer gliadin peptide complex was then added at 1.2×10⁴ beads/micro L of solution and immobilized for 60 minutes with shaking at room temperature on 96-well plates (Sigma Aldrich, Cat No. M2686). Final concentration of the HLA-DQ2.5/33mer gliadin peptide complex was 0.375 micro g/mL. The plates were washed with blocking buffer, and serially diluted anti-HLA-DQ antibodies were added and incubated for 60 minutes with shaking at room temperature. S2 TCR tetramer-PE was then added to HLA-DQ2.5/33mer gliadin peptide-coated beads and incubated for 60 minutes with shaking at 4 degrees C. and washed with blocking buffer. Final concentration of S2 TCR tetramer-PE was 2.0 micro g/mL. Data acquisition was performed on a LSR Fortessa (Becton Dickinson), followed by analysis using FlowJo software (Tree Star) and GraphPad Prism software (GraphPad).

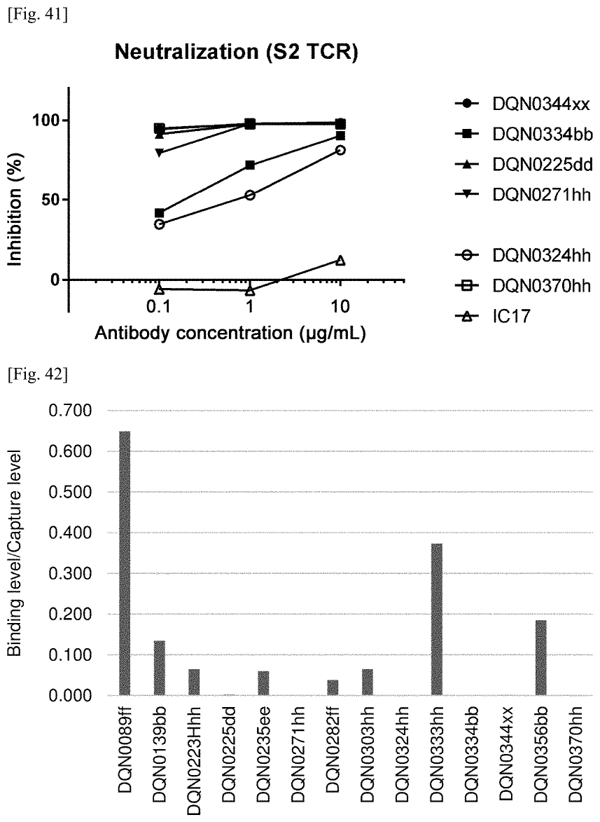

As shown in FIG. 41, the antibodies have neutralizing activity against the binding between gliadin-bound HLA-DQ2.5 and S2 TCR. Thus, it was indicated that the antibodies of the present invention can block the interaction between HLA-DQ2.5 and an HLA-DQ2.5-restricted CD4+ T cell.

### Example 14

Biacore Analysis for Binding Affinity Evaluation of Anti-HLA-DQ2.5 Antibodies.

The affinity of anti-HLA-DQ2.5 antibodies binding to human HLA-DQ2.5/33mer gliadin peptide complex at pH 7.4 were determined at 37 degrees C. using Biacore 8K instrument (GE Healthcare). Anti-human Fc (GE Healthcare) was immobilized onto all flow cells of a CM4 sensor chip using amine coupling kit (GE Healthcare). All antibodies and analytes were prepared in ACES pH 7.4 containing 20 mM ACES, 150 mM NaCl, 0.05% Tween 20, 0.005% NaN3. Each antibody was captured onto the sensor surface by anti-human Fc. Antibody capture levels were aimed at 200 resonance unit (RU). Recombinant human HLA-DQ2.5/33mer gliadin peptide complex was injected at 50 to 800 nM prepared by two-fold serial dilution, followed by dissociation. Sensor surface was regenerated each cycle with 3M MgCl2. Binding affinity were determined by processing and fitting the data to 1:1 binding model using Biacore 8K Evaluation software (GE Healthcare).

The affinity of anti-HLA-DQ2.5 antibodies binding to HLA-DQ2.5/33mer gliadin peptide complex are shown in Table 8.

TABLE 8

| Ab name | ka (M⁻¹s⁻¹) | kd (s⁻¹) | KD (M) |
|---|---|---|---|
| DQN0089ff | 2.28E+05 | 9.86E−03 | 4.31E−08 |
| DQN0139bb | 6.96E+04 | 4.63E−04 | 6.65E−09 |
| DQN0223hh | 1.39E+05 | 2.94E−02 | 2.11E−07 |
| DQN0225dd | 2.40E+05 | 1.89E−03 | 7.88E−09 |
| DQN0235ee | 9.16E+04 | 1.72E−03 | 1.87E−08 |
| DQN0271hh | 2.02E+05 | 1.23E−03 | 6.07E−09 |
| DQN0282ff | 6.63E+04 | 7.57E−04 | 1.14E−08 |
| DQN0303hh | 7.38E+04 | 3.08E−04 | 4.17E−09 |
| DQN0324hh | 6.43E+04 | 4.06E−04 | 6.32E−09 |
| DQN0333hh | 7.39E+04 | 1.61E−03 | 2.18E−08 |
| DQN0334bb | 1.03E+05 | 5.91E−04 | 5.72E−09 |
| DQN0344xx | 1.75E+05 | 1.54E−03 | 8.83E−09 |
| DQN0356bb | 1.11E+05 | 4.49E−02 | 4.04E−07 |
| DQN0370hh | 2.52E+05 | 1.13E−03 | 4.47E−09 |

### Example 15

Assessment of HLA-DQ2.5/Invariant Chain Complex Binding for Anti-HLA-DQ2.5 Antibodies.

The binding response of anti-HLA-DQ2.5 antibodies to human HLA-DQ2.5/invariant chain complex at pH 7.4 were determined at 25 degrees C. using Biacore 8K instrument (GE Healthcare). Anti-human Fc (GE Healthcare) was immobilized onto all flow cells of a CM4 sensor chip using amine coupling kit (GE Healthcare). All antibodies and analytes were prepared in ACES pH 7.4 containing 20 mM ACES, 150 mM NaCl, 0.05% Tween 20, 0.005% NaN3. Each antibody was captured onto the sensor surface by anti-human Fc. Antibody capture levels were aimed at 200 resonance unit (RU). Recombinant human HLA-DQ/invariant chain complex was injected at 100 nM, followed by dissociation. Sensor surface was regenerated each cycle with 3M MgCl₂.

The binding level of anti-HLA-DQ2.5 antibodies towards human HLA-DQ2.5/invariant chain were monitored from the binding response. Binding levels were normalized to the capture level of corresponding anti-HLA-DQ2.5 antibodies.

Since the amount of the antibody captured on the tip varied, the ratio of the binding level to the capture level was used for evaluation of the binding activity. As shown in FIG. 42, no significant binding towards HLA-DQ2.5/invariant chain was observed for the anti-HLA-DQ2.5 antibodies. Thus, it is thought that the antibodies of the present invention do not specifically bind to the complex of the invariant chain and HLA-DQ.

### Reference Example 1

Preparation of Delta-GK Fc

The human IgG4-derived delta-GK Fc fragment was expressed using the FreeStyle™ 293 expression system (Invitrogen). The expressed Fc fragment was purified from the harvested cell culture media by affinity chromatography (Mab Select SuRe, GE). In the final step, we exchanged the buffer to D-PBS(−).

### Reference Example 2

Generation of Anti-Delta-GK Antibody

Anti-delta-GK antibodies were prepared, selected, and assayed as described below. NZW rabbits were immunized intradermally with the human IgG4-derived delta-GK Fc fragment expressed in Example 1 (100-200 micro g/dose/head). The dose was repeatedly given 6 times over a 3-month period followed by blood and spleen collection. For B-cell selection, an IgG4 delta-GK antibody (an IgG4 antibody with genetically deleted IgG4 C-terminal GK) and a wild type IgG4 antibody were prepared. Delta-GK specific B-cells were sorted using a cell sorter and then plated and cultured according to the procedure described in WO2016098356A1. After cultivation, the B-cell culture supernatants were harvested for further analysis and the corresponding B-cell pellets were cryopreserved.

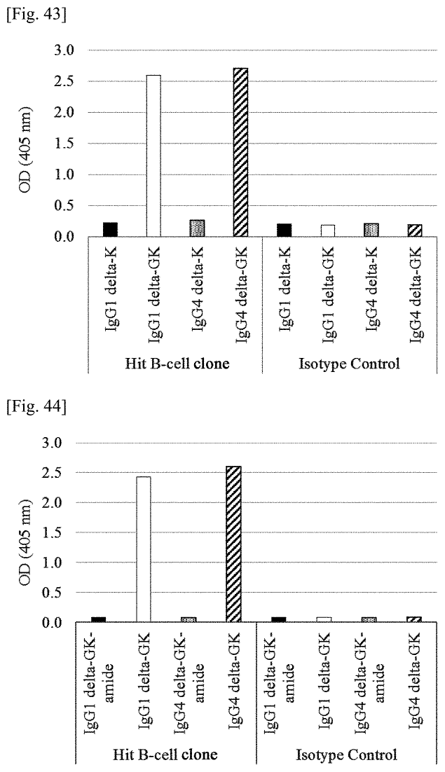

Specific binding to IgG delta-GK was evaluated by ELISA using the B-cell culture supernatants. In this primary screening, four types of antibodies were used as antigens in order to evaluate the binding specificity against the delta-GK C-terminal sequence: an IgG1 antibody with genetically deleted IgG1 C-terminal K (IgG1 delta-K), an IgG1 antibody with genetically deleted IgG1 C-terminal GK (IgG1 delta-GK), an IgG4 antibody with genetically deleted IgG4 C-terminal K (IgG4 delta-K) and an IgG4 antibody with genetically deleted IgG4 C-terminal GK (IgG4 delta-GK). The results showed that only one culture supernatant sample from a single B cell clone demonstrated specific binding to both IgG1 delta-GK and IgG4 delta-GK (FIG. 43).

Delta-GK Fc is more structurally similar to delta-GK-amide Fc than to delta-K Fc. We have also characterized specific binding to delta-GK Fc and delta-GK-amide Fc using the aforementioned selected culture supernatant from the positive B cell clone. IgG1 delta-GK-amide and IgG4 delta-GK-amide were prepared by PAM treatment with IgG1 delta-K or IgG4 delta-K mentioned above and were purified by conventional method. In this secondary screening, four types of antibodies were used as antigens in an ELISA assay to evaluate the binding specificity against the delta-GK C-terminal sequence: IgG1 delta-GK, IgG1 delta-GK-amide, IgG4 delta-GK, and IgG4 delta-GK-amide. Surprisingly, the tested single B cell culture supernatant showed extremely high specificity against delta-GK molecules (FIG. 44).

Based on these screening results, the RNA of the selected clone was extracted from its cryopreserved cell pellet using ZR-96 Quick-RNA kits (ZYMO RESEARCH, Cat No. R1053). DNA encoding the antibody heavy chain variable region in the antibody produced by the selected clone was obtained and amplified by reverse transcription PCR then recombined with DNA encoding the rabbit IgG heavy chain constant region (SEQ ID NO: 140). DNA encoding the antibody light chain variable regions was also obtained and amplified by reverse transcription PCR then recombined with DNA encoding the rabbit Igk light chain constant region (SEQ ID NO: 141). An anti-delta-GK antibody, termed “YG55”, which has two heavy chains and two light chains, was produced from these recombinants. The VH, VL and HVRs sequences of the heavy and light chains are described in Table 9. YG55 was expressed using the FreeStyle™ 293 expression system and purified from the culture supernatants.

### Reference Example 3

Characterization of Anti-Delta-GK Monoclonal Antibody YG55

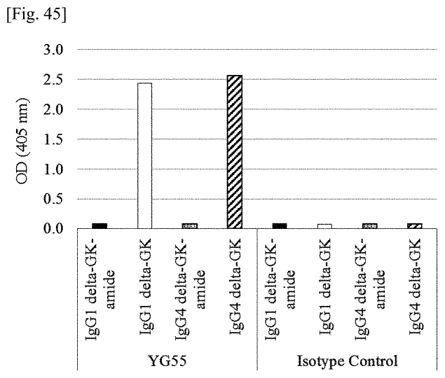

After gene cloning and antibody expression, the specificity of YG55 was assessed by the ELISA assay as described above in the secondary screening. The antibody gene cloning was successful, resulting in YG55 retaining the same specificity shown by the hit (positive) B-cell clone (FIG. 45). This highly specific binding was also confirmed by surface plasmon resonance assay. The specific binding motif and its epitope were identified by crystal structure analysis.

TABLE 9

| | SEQ ID NO: | | | | | | | |
|---|---|---|---|---|---|---|---|---|
| Antibody | | HVR- | HVR- | HVR- | | HVR- | HVR- | HVR- |
| Name | VH | H1 | H2 | H3 | VL | L1 | L2 | L3 |
| YG55 | 174 | 175 | 176 | 177 | 178 | 179 | 180 | 181 |

1-16. (canceled)

17. An anti-HLA-DQ2.5 antibody that has binding activity to HLA-DQ2.5 in the form of a complex with a gluten peptide (HLA-DQ2.5/gluten peptide complex) and substantially no binding activity to HLA-DQ8.

18. The antibody of claim 17, wherein the gluten peptide is a first gluten peptide selected from the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide, and a secalin 2 peptide.

19. The antibody of claim 17, wherein the antibody blocks interaction between the HLA-DQ2.5/gluten peptide complex and an HLA-DQ2.5/gluten peptide-restricted CD4+ T cell.

20. The antibody of claim 17, wherein the antibody has substantially no binding activity to HLA-DQ5.1, HLA-DQ6.3, and HLA-DQ7.3.

21. The antibody of claim 17, wherein the antibody has substantially no binding activity to HLA-DR and HLA-DP.

22. The antibody of claim 17, wherein the antibody has substantially no binding activity to a complex of HLA-DQ2.5 with an invariant chain (HLA-DQ2.5/invariant chain complex).

23. The antibody of claim 17, wherein the antibody has binding activity to HLA-DQ2.2 and substantially no binding activity to HLA-DQ7.5.

24. The antibody of claim 17, wherein the antibody has binding activity to HLA-DQ7.5 and substantially no binding activity to HLA-DQ2.2.

25. The antibody of claim 17, wherein the antibody has substantially no binding activity to HLA-DQ2.2 and HLA-DQ7.5.

26. The antibody of claim 25, wherein the antibody binds more strongly to HLA-DQ2.5 in the form of a complex with a gluten peptide than (a) to HLA-DQ2.5 alone, and (b) to a complex of HLA-DQ2.5 with a peptide other than a gluten peptide.

27. An antibody of any one of (1) to (12) below:
(1) an antibody comprising the HCDR1 sequence of SEQ ID NO: 13, the HCDR2 sequence of SEQ ID NO: 25, the HCDR3 sequence of SEQ ID NO: 37, the LCDR1 sequence of SEQ ID NO: 61, the LCDR2 sequence of SEQ ID NO: 73, and the LCDR3 sequence of SEQ ID NO: 85;
(2) an antibody comprising the HCDR1 sequence of SEQ ID NO: 14, the HCDR2 sequence of SEQ ID NO: 26, the HCDR3 sequence of SEQ ID NO: 38, the LCDR1 sequence of SEQ ID NO: 62, the LCDR2 sequence of SEQ ID NO: 74, and the LCDR3 sequence of SEQ ID NO: 86;
(3) an antibody comprising the HCDR1 sequence of SEQ ID NO: 15, the HCDR2 sequence of SEQ ID NO: 27, the HCDR3 sequence of SEQ ID NO: 39, the LCDR1 sequence of SEQ ID NO: 63, the LCDR2 sequence of SEQ ID NO: 75, and the LCDR3 sequence of SEQ ID NO: 87;
(4) an antibody comprising the HCDR1 sequence of SEQ ID NO: 16, the HCDR2 sequence of SEQ ID NO: 28, the HCDR3 sequence of SEQ ID NO: 40, the LCDR1 sequence of SEQ ID NO: 64, the LCDR2 sequence of SEQ ID NO: 76, and the LCDR3 sequence of SEQ ID NO: 88;
(5) an antibody comprising the HCDR1 sequence of SEQ ID NO: 17, the HCDR2 sequence of SEQ ID NO: 29, the HCDR3 sequence of SEQ ID NO: 41, the LCDR1 sequence of SEQ ID NO: 65, the LCDR2 sequence of SEQ ID NO: 77, and the LCDR3 sequence of SEQ ID NO: 89;
(6) an antibody comprising the HCDR1 sequence of SEQ ID NO: 18, the HCDR2 sequence of SEQ ID NO: 30, the HCDR3 sequence of SEQ ID NO: 42, the LCDR1 sequence of SEQ ID NO: 66, the LCDR2 sequence of SEQ ID NO: 78, and the LCDR3 sequence of SEQ ID NO: 90;
(7) an antibody comprising the HCDR1 sequence of SEQ ID NO: 19, the HCDR2 sequence of SEQ ID NO: 31, the HCDR3 sequence of SEQ ID NO: 43, the LCDR1 sequence of SEQ ID NO: 67, the LCDR2 sequence of SEQ ID NO: 79, and the LCDR3 sequence of SEQ ID NO: 91;
(8) an antibody comprising the HCDR1 sequence of SEQ ID NO: 20, the HCDR2 sequence of SEQ ID NO: 32, the HCDR3 sequence of SEQ ID NO: 44, the LCDR1 sequence of SEQ ID NO: 68, the LCDR2 sequence of SEQ ID NO: 80, and the LCDR3 sequence of SEQ ID NO: 92;
(9) an antibody comprising the HCDR1 sequence of SEQ ID NO: 146, the HCDR2 sequence of SEQ ID NO: 150, the HCDR3 sequence of SEQ ID NO: 154, the LCDR1 sequence of SEQ ID NO: 162, the LCDR2 sequence of SEQ ID NO: 166, and the LCDR3 sequence of SEQ ID NO: 170;
(10) an antibody comprising the HCDR1 sequence of SEQ ID NO: 147, the HCDR2 sequence of SEQ ID NO: 151, the HCDR3 sequence of SEQ ID NO: 155, the LCDR1 sequence of SEQ ID NO: 163, the LCDR2 sequence of SEQ ID NO: 167, and the LCDR3 sequence of SEQ ID NO: 171;
(11) an antibody comprising the HCDR1 sequence of SEQ ID NO: 148, the HCDR2 sequence of SEQ ID NO: 152, the HCDR3 sequence of SEQ ID NO: 156, the LCDR1 sequence of SEQ ID NO: 164, the LCDR2 sequence of SEQ ID NO: 168, and the LCDR3 sequence of SEQ ID NO: 172;
(12) an antibody comprising the HCDR1 sequence of SEQ ID NO: 149, the HCDR2 sequence of SEQ ID NO: 153, the HCDR3 sequence of SEQ ID NO: 157, the LCDR1 sequence of SEQ ID NO: 165, the LCDR2 sequence of SEQ ID NO: 169, and the LCDR3 sequence of SEQ ID NO: 173.

28. An anti-HLA-DQ2.5 antibody that has binding activity to the beta chain of HLA-DQ2.5 and blocks interaction between (a) an HLA-DQ2.5/gluten peptide complex and (b) an HLA-DQ2.5/gluten peptide-restricted CD4+ T cell.

29. An anti-HLA-DQ2.5 antibody that has binding activity to the alpha chain of HLA-DQ2.5 and blocks the interaction between (a) an HLA-DQ2.5/gluten peptide complex and (b) an HLA-DQ2.5/gluten peptide-restricted CD4+ T cell.

30. An antibody that
(a) binds to the same HLA-DQ2.5 epitope bound by an antibody of any one of (1)-(12) below, or
(b) competes for binding to HLA-DQ2.5 or to a complex of a gluten peptide and HLA-DQ2.5 with an antibody of any one of (1)-(12) below:
(1) an antibody comprising the HCDR1 sequence of SEQ ID NO: 13, the HCDR2 sequence of SEQ ID NO: 25, the HCDR3 sequence of SEQ ID NO: 37, the LCDR1 sequence of SEQ ID NO: 61, the LCDR2 sequence of SEQ ID NO: 73, and the LCDR3 sequence of SEQ ID NO: 85;
(2) an antibody comprising the HCDR1 sequence of SEQ ID NO: 14, the HCDR2 sequence of SEQ ID NO: 26, the HCDR3 sequence of SEQ ID NO: 38, the LCDR1 sequence of SEQ ID NO: 62, the LCDR2 sequence of SEQ ID NO: 74, and the LCDR3 sequence of SEQ ID NO: 86;
(3) an antibody comprising the HCDR1 sequence of SEQ ID NO: 15, the HCDR2 sequence of SEQ ID NO: 27, the HCDR3 sequence of SEQ ID NO: 39, the LCDR1 sequence of SEQ ID NO: 63, the LCDR2 sequence of SEQ ID NO: 75, and the LCDR3 sequence of SEQ ID NO: 87;
(4) an antibody comprising the HCDR1 sequence of SEQ ID NO: 16, the HCDR2 sequence of SEQ ID NO: 28, the HCDR3 sequence of SEQ ID NO: 40, the LCDR1 sequence of SEQ ID NO: 64, the LCDR2 sequence of SEQ ID NO: 76, and the LCDR3 sequence of SEQ ID NO: 88;
(5) an antibody comprising the HCDR1 sequence of SEQ ID NO: 17, the HCDR2 sequence of SEQ ID NO: 29, the HCDR3 sequence of SEQ ID NO: 41, the LCDR1 sequence of SEQ ID NO: 65, the LCDR2 sequence of SEQ ID NO: 77, and the LCDR3 sequence of SEQ ID NO: 89;
(6) an antibody comprising the HCDR1 sequence of SEQ ID NO: 18, the HCDR2 sequence of SEQ ID NO: 30, the HCDR3 sequence of SEQ ID NO: 42, the LCDR1 sequence of SEQ ID NO: 66, the LCDR2 sequence of SEQ ID NO: 78, and the LCDR3 sequence of SEQ ID NO: 90;
(7) an antibody comprising the HCDR1 sequence of SEQ ID NO: 19, the HCDR2 sequence of SEQ ID NO: 31, the HCDR3 sequence of SEQ ID NO: 43, the LCDR1 sequence of SEQ ID NO: 67, the LCDR2 sequence of SEQ ID NO: 79, and the LCDR3 sequence of SEQ ID NO: 91;
(8) an antibody comprising the HCDR1 sequence of SEQ ID NO: 20, the HCDR2 sequence of SEQ ID NO: 32, the HCDR3 sequence of SEQ ID NO: 44, the LCDR1 sequence of SEQ ID NO: 68, the LCDR2 sequence of SEQ ID NO: 80, and the LCDR3 sequence of SEQ ID NO: 92;
(9) an antibody comprising the HCDR1 sequence of SEQ ID NO: 146, the HCDR2 sequence of SEQ ID NO: 150, the HCDR3 sequence of SEQ ID NO: 154, the LCDR1 sequence of SEQ ID NO: 162, the LCDR2 sequence of SEQ ID NO: 166, and the LCDR3 sequence of SEQ ID NO: 170;
(10) an antibody comprising the HCDR1 sequence of SEQ ID NO: 147, the HCDR2 sequence of SEQ ID NO: 151, the HCDR3 sequence of SEQ ID NO: 155, the LCDR1 sequence of SEQ ID NO: 163, the LCDR2 sequence of SEQ ID NO: 167, and the LCDR3 sequence of SEQ ID NO: 171;
(11) an antibody comprising the HCDR1 sequence of SEQ ID NO: 148, the HCDR2 sequence of SEQ ID NO: 152, the HCDR3 sequence of SEQ ID NO: 156, the LCDR1 sequence of SEQ ID NO: 164, the LCDR2 sequence of SEQ ID NO: 168, and the LCDR3 sequence of SEQ ID NO: 172;
(12) an antibody comprising the HCDR1 sequence of SEQ ID NO: 149, the HCDR2 sequence of SEQ ID NO: 153, the HCDR3 sequence of SEQ ID NO: 157, the LCDR1 sequence of SEQ ID NO: 165, the LCDR2 sequence of SEQ ID NO: 169, and the LCDR3 sequence of SEQ ID NO: 173.

31. A pharmaceutical formulation comprising the antibody of claim 17 and a pharmaceutically acceptable carrier.

32. The antibody of claim 18, wherein the antibody also has binding activity to a complex of HLA-DQ2.5 and a second gluten peptide, wherein the second gluten peptide is different from the first gluten peptide and is selected from the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide, and a secalin 2 peptide.

33. The antibody of claim 32, wherein the antibody also has binding activity to a complex of HLA-DQ2.5 and a third gluten peptide, wherein the third gluten peptide is different from the first and second gluten peptides and is selected from the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide, and a secalin 2 peptide.

34. The antibody of claim 33, wherein the antibody also has binding activity to a complex of HLA-DQ2.5 and a fourth gluten peptide, wherein the fourth gluten peptide is different from the first, second, and third gluten peptides and is selected from the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide, and a secalin 2 peptide.

35. The antibody of claim 34, wherein the antibody also has binding activity to a complex of HLA-DQ2.5 and a fifth gluten peptide, wherein the fifth gluten peptide is different from the first, second, third, and fourth gluten peptides and is selected from the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide, and a secalin 2 peptide.

36. The antibody of claim 35, wherein the antibody also has binding activity to a complex of HLA-DQ2.5 and a sixth gluten peptide, wherein the sixth gluten peptide is different from the first, second, third, fourth, and fifth gluten peptides and is selected from the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide, and a secalin 2 peptide.

37. The antibody of claim 36, wherein the antibody also has binding activity to a complex of HLA-DQ2.5 and a seventh gluten peptide, wherein the seventh gluten peptide is different from the first, second, third, fourth, fifth, and sixth gluten peptides and is selected from the group consisting of a 33mer gliadin peptide, an alpha 1 gliadin peptide, an alpha 1b gliadin peptide, an alpha 2 gliadin peptide, an omega 1 gliadin peptide, an omega 2 gliadin peptide, a secalin 1 peptide, and a secalin 2 peptide.

38. The antibody of claim 17, wherein the antibody has binding activity to each of the following:
a complex of HLA-DQ2.5 and a 33mer gliadin peptide,
a complex of HLA-DQ2.5 and an alpha 1 gliadin peptide,
a complex of HLA-DQ2.5 and an alpha 1b gliadin peptide,
a complex of HLA-DQ2.5 and an alpha 2 gliadin peptide,
a complex of HLA-DQ2.5 and an omega 1 gliadin peptide,
a complex of HLA-DQ2.5 and an omega 2 gliadin peptide,
a complex of HLA-DQ2.5 and a secalin 1 peptide, and
a complex of HLA-DQ2.5 and a secalin 2 peptide.

39. The antibody of claim 26, wherein the antibody binds more strongly to an HLA-DQ2.5/gluten peptide complex selected from the group consisting of:
a complex of HLA-DQ2.5 and a 33mer gliadin peptide,
a complex of HLA-DQ2.5 and an alpha 1 gliadin peptide,
a complex of HLA-DQ2.5 and an alpha 1b gliadin peptide,
a complex of HLA-DQ2.5 and an alpha 2 gliadin peptide,
a complex of HLA-DQ2.5 and an omega 1 gliadin peptide,
a complex of HLA-DQ2.5 and an omega 2 gliadin peptide,
a complex of HLA-DQ2.5 and a secalin 1 peptide, and
a complex of HLA-DQ2.5 and a secalin 2 peptide,
than to a complex or a cell selected from the group consisting of:
a complex of HLA-DQ2.5 and a CLIP peptide,
a complex of HLA-DQ2.5 and a Salmonella peptide,
a complex of HLA-DQ2.5 and a Mycobacterium bovis peptide,
a complex of HLA-DQ2.5 and a Hepatitis B virus peptide, and
an HLA-DQ2.5 positive PBMC-B cell.

40. An antibody that has binding activity to an HLA-DQ2.5/gluten peptide complex selected from the group consisting of:
a complex of HLA-DQ2.5 and a 33mer gliadin peptide,
a complex of HLA-DQ2.5 and an alpha 1 gliadin peptide,
a complex of HLA-DQ2.5 and an alpha 1b gliadin peptide,
a complex of HLA-DQ2.5 and an alpha 2 gliadin peptide,
a complex of HLA-DQ2.5 and an omega 1 gliadin peptide,
a complex of HLA-DQ2.5 and an omega 2 gliadin peptide,
a complex of HLA-DQ2.5 and a secalin 1 peptide, and
a complex of HLA-DQ2.5 and a secalin 2 peptide,
and substantially no binding activity to a complex or a cell selected from the group consisting of:
a complex of HLA-DQ2.5 and a CLIP peptide,
a complex of HLA-DQ2.5 and a Salmonella peptide,
a complex of HLA-DQ2.5 and a Mycobacterium bovis peptide,
a complex of HLA-DQ2.5 and a Hepatitis B virus peptide, and
an HLA-DQ2.5 positive PBMC-B cell;
wherein the antibody blocks interaction between the HLA-DQ2.5/gluten peptide complex and an HLA-DQ2.5/gluten peptide-restricted CD4+ T cell.

41. A method for treating a subject having celiac disease, the method comprising administering to the subject an effective amount of the pharmaceutical formulation of claim 31, thereby treating the subject.

42. One or more nucleic acid molecules encoding an antibody of any one of (1)-(12):
(1) an antibody comprising the HCDR1 sequence of SEQ ID NO: 13, the HCDR2 sequence of SEQ ID NO: 25, the HCDR3 sequence of SEQ ID NO: 37, the LCDR1 sequence of SEQ ID NO: 61, the LCDR2 sequence of SEQ ID NO: 73, and the LCDR3 sequence of SEQ ID NO: 85;
(2) an antibody comprising the HCDR1 sequence of SEQ ID NO: 14, the HCDR2 sequence of SEQ ID NO: 26, the HCDR3 sequence of SEQ ID NO: 38, the LCDR1 sequence of SEQ ID NO: 62, the LCDR2 sequence of SEQ ID NO: 74, and the LCDR3 sequence of SEQ ID NO: 86;
(3) an antibody comprising the HCDR1 sequence of SEQ ID NO: 15, the HCDR2 sequence of SEQ ID NO: 27, the HCDR3 sequence of SEQ ID NO: 39, the LCDR1 sequence of SEQ ID NO: 63, the LCDR2 sequence of SEQ ID NO: 75, and the LCDR3 sequence of SEQ ID NO: 87;
(4) an antibody comprising the HCDR1 sequence of SEQ ID NO: 16, the HCDR2 sequence of SEQ ID NO: 28, the HCDR3 sequence of SEQ ID NO: 40, the LCDR1 sequence of SEQ ID NO: 64, the LCDR2 sequence of SEQ ID NO: 76, and the LCDR3 sequence of SEQ ID NO: 88;
(5) an antibody comprising the HCDR1 sequence of SEQ ID NO: 17, the HCDR2 sequence of SEQ ID NO: 29, the HCDR3 sequence of SEQ ID NO: 41, the LCDR1 sequence of SEQ ID NO: 65, the LCDR2 sequence of SEQ ID NO: 77, and the LCDR3 sequence of SEQ ID NO: 89;
(6) an antibody comprising the HCDR1 sequence of SEQ ID NO: 18, the HCDR2 sequence of SEQ ID NO: 30, the HCDR3 sequence of SEQ ID NO: 42, the LCDR1 sequence of SEQ ID NO: 66, the LCDR2 sequence of SEQ ID NO: 78, and the LCDR3 sequence of SEQ ID NO: 90;
(7) an antibody comprising the HCDR1 sequence of SEQ ID NO: 19, the HCDR2 sequence of SEQ ID NO: 31, the HCDR3 sequence of SEQ ID NO: 43, the LCDR1 sequence of SEQ ID NO: 67, the LCDR2 sequence of SEQ ID NO: 79, and the LCDR3 sequence of SEQ ID NO: 91;
(8) an antibody comprising the HCDR1 sequence of SEQ ID NO: 20, the HCDR2 sequence of SEQ ID NO: 32, the HCDR3 sequence of SEQ ID NO: 44, the LCDR1 sequence of SEQ ID NO: 68, the LCDR2 sequence of SEQ ID NO: 80, and the LCDR3 sequence of SEQ ID NO: 92;
(9) an antibody comprising the HCDR1 sequence of SEQ ID NO: 146, the HCDR2 sequence of SEQ ID NO: 150, the HCDR3 sequence of SEQ ID NO: 154, the LCDR1 sequence of SEQ ID NO: 162, the LCDR2 sequence of SEQ ID NO: 166, and the LCDR3 sequence of SEQ ID NO: 170;
(10) an antibody comprising the HCDR1 sequence of SEQ ID NO: 147, the HCDR2 sequence of SEQ ID NO: 151, the HCDR3 sequence of SEQ ID NO: 155, the LCDR1 sequence of SEQ ID NO: 163, the LCDR2 sequence of SEQ ID NO: 167, and the LCDR3 sequence of SEQ ID NO: 171;
(11) an antibody comprising the HCDR1 sequence of SEQ ID NO: 148, the HCDR2 sequence of SEQ ID NO: 152, the HCDR3 sequence of SEQ ID NO: 156, the LCDR1 sequence of SEQ ID NO: 164, the LCDR2 sequence of SEQ ID NO: 168, and the LCDR3 sequence of SEQ ID NO: 172;
(12) an antibody comprising the HCDR1 sequence of SEQ ID NO: 149, the HCDR2 sequence of SEQ ID NO: 153, the HCDR3 sequence of SEQ ID NO: 157, the LCDR1 sequence of SEQ ID NO: 165, the LCDR2 sequence of SEQ ID NO: 169, and the LCDR3 sequence of SEQ ID NO: 173.

43. A host cell comprising the one or more nucleic acid molecules of claim 42.

44. A method for producing an antibody, the method comprising expressing the one or more nucleic acid molecules of claim 42.

45. The antibody of claim 17, wherein the gluten peptide is a first gluten peptide selected from the group consisting of a 33mer gliadin peptide, an alpha 1 gliadin peptide, an alpha 1b gliadin peptide, an alpha 2 gliadin peptide, an omega 1 gliadin peptide, a secalin 1 peptide, and a secalin 2 peptide.

46. The antibody of claim 45, wherein the antibody also has binding activity to a complex of HLA-DQ2.5 and a second gluten peptide, wherein the second gluten peptide is different from the first gluten peptide and is selected from the group consisting of a 33mer gliadin peptide, an alpha 1 gliadin peptide, an alpha 1b gliadin peptide, an alpha 2 gliadin peptide, an omega 1 gliadin peptide, a secalin 1 peptide, and a secalin 2 peptide.

47. The antibody of claim 46, wherein the antibody also has binding activity to a complex of HLA-DQ2.5 and a third gluten peptide, wherein the third gluten peptide is different from the first and second gluten peptides and is selected from the group consisting of a 33mer gliadin peptide, an alpha 1 gliadin peptide, an alpha 1b gliadin peptide, an alpha 2 gliadin peptide, an omega 1 gliadin peptide, a secalin 1 peptide, and a secalin 2 peptide.

48. The antibody of claim 47, wherein the antibody also has binding activity to a complex of HLA-DQ2.5 and a fourth gluten peptide, wherein the fourth gluten peptide is different from the first, second, and third gluten peptides and is selected from the group consisting of a 33mer gliadin peptide, an alpha 1 gliadin peptide, an alpha 1b gliadin peptide, an alpha 2 gliadin peptide, an omega 1 gliadin peptide, a secalin 1 peptide, and a secalin 2 peptide.

49. The antibody of claim 48, wherein the antibody also has binding activity to a complex of HLA-DQ2.5 and a fifth gluten peptide, wherein the fifth gluten peptide is different from the first, second, third, and fourth gluten peptides and is selected from the group consisting of a 33mer gliadin peptide, an alpha 1 gliadin peptide, an alpha 1b gliadin peptide, an alpha 2 gliadin peptide, an omega 1 gliadin peptide, a secalin 1 peptide, and a secalin 2 peptide.

50. The antibody of claim 49, wherein the antibody also has binding activity to a complex of HLA-DQ2.5 and a sixth gluten peptide, wherein the sixth gluten peptide is different from the first, second, third, fourth, and fifth gluten peptides and is selected from the group consisting of a 33mer gliadin peptide, an alpha 1 gliadin peptide, an alpha 1b gliadin peptide, an alpha 2 gliadin peptide, an omega 1 gliadin peptide, a secalin 1 peptide, and a secalin 2 peptide.

51. The antibody of claim 17, wherein the antibody has binding activity to each of the following:
a complex of HLA-DQ2.5 and a 33mer gliadin peptide,
a complex of HLA-DQ2.5 and an alpha 1 gliadin peptide,
a complex of HLA-DQ2.5 and an alpha 1b gliadin peptide,
a complex of HLA-DQ2.5 and an alpha 2 gliadin peptide,
a complex of HLA-DQ2.5 and an omega 1 gliadin peptide,
a complex of HLA-DQ2.5 and a secalin 1 peptide, and
a complex of HLA-DQ2.5 and a secalin 2 peptide.

52. The antibody of claim 27, wherein the antibody has binding activity to HLA-DQ2.5 in the form of a complex with a gluten peptide (HLA-DQ2.5/gluten peptide complex).

53. The antibody of claim 27, wherein the antibody comprises:
the HCDR1 sequence of SEQ ID NO: 13, the HCDR2 sequence of SEQ ID NO: 25, the HCDR3 sequence of SEQ ID NO: 37, the LCDR1 sequence of SEQ ID NO: 61, the LCDR2 sequence of SEQ ID NO: 73, and the LCDR3 sequence of SEQ ID NO: 85.

54. The antibody of claim 27, wherein the antibody comprises:
the HCDR1 sequence of SEQ ID NO: 14, the HCDR2 sequence of SEQ ID NO: 26, the HCDR3 sequence of SEQ ID NO: 38, the LCDR1 sequence of SEQ ID NO: 62, the LCDR2 sequence of SEQ ID NO: 74, and the LCDR3 sequence of SEQ ID NO: 86.

55. The antibody of claim 27, wherein the antibody comprises:
the HCDR1 sequence of SEQ ID NO: 15, the HCDR2 sequence of SEQ ID NO: 27, the HCDR3 sequence of SEQ ID NO: 39, the LCDR1 sequence of SEQ ID NO: 63, the LCDR2 sequence of SEQ ID NO: 75, and the LCDR3 sequence of SEQ ID NO: 87.

56. The antibody of claim 27, wherein the antibody comprises:
the HCDR1 sequence of SEQ ID NO: 16, the HCDR2 sequence of SEQ ID NO: 28, the HCDR3 sequence of SEQ ID NO: 40, the LCDR1 sequence of SEQ ID NO: 64, the LCDR2 sequence of SEQ ID NO: 76, and the LCDR3 sequence of SEQ ID NO: 88.

57. The antibody of claim 27, wherein the antibody comprises:
the HCDR1 sequence of SEQ ID NO: 17, the HCDR2 sequence of SEQ ID NO: 29, the HCDR3 sequence of SEQ ID NO: 41, the LCDR1 sequence of SEQ ID NO: 65, the LCDR2 sequence of SEQ ID NO: 77, and the LCDR3 sequence of SEQ ID NO: 89.

58. The antibody of claim 27, wherein the antibody comprises:
the HCDR1 sequence of SEQ ID NO: 18, the HCDR2 sequence of SEQ ID NO: 30, the HCDR3 sequence of SEQ ID NO: 42, the LCDR1 sequence of SEQ ID NO: 66, the LCDR2 sequence of SEQ ID NO: 78, and the LCDR3 sequence of SEQ ID NO: 90.

59. The antibody of claim 27, wherein the antibody comprises:
the HCDR1 sequence of SEQ ID NO: 19, the HCDR2 sequence of SEQ ID NO: 31, the HCDR3 sequence of SEQ ID NO: 43, the LCDR1 sequence of SEQ ID NO: 67, the LCDR2 sequence of SEQ ID NO: 79, and the LCDR3 sequence of SEQ ID NO: 91.

60. The antibody of claim 27, wherein the antibody comprises:
the HCDR1 sequence of SEQ ID NO: 20, the HCDR2 sequence of SEQ ID NO: 32, the HCDR3 sequence of SEQ ID NO: 44, the LCDR1 sequence of SEQ ID NO: 68, the LCDR2 sequence of SEQ ID NO: 80, and the LCDR3 sequence of SEQ ID NO: 92.

61. The antibody of claim 27, wherein the antibody comprises:
HCDR1 sequence of SEQ ID NO: 146, the HCDR2 sequence of SEQ ID NO: 150, the HCDR3 sequence of SEQ ID NO: 154, the LCDR1 sequence of SEQ ID NO: 162, the LCDR2 sequence of SEQ ID NO: 166, and the LCDR3 sequence of SEQ ID NO: 170.

62. The antibody of claim 27, wherein the antibody comprises:
the HCDR1 sequence of SEQ ID NO: 147, the HCDR2 sequence of SEQ ID NO: 151, the HCDR3 sequence of SEQ ID NO: 155, the LCDR1 sequence of SEQ ID NO: 163, the LCDR2 sequence of SEQ ID NO: 167, and the LCDR3 sequence of SEQ ID NO: 171.

63. The antibody of claim 27, wherein the antibody comprises:
the HCDR1 sequence of SEQ ID NO: 148, the HCDR2 sequence of SEQ ID NO: 152, the HCDR3 sequence of SEQ ID NO: 156, the LCDR1 sequence of SEQ ID NO: 164, the LCDR2 sequence of SEQ ID NO: 168, and the LCDR3 sequence of SEQ ID NO: 172.

64. The antibody of claim 27, wherein the antibody comprises:
the HCDR1 sequence of SEQ ID NO: 149, the HCDR2 sequence of SEQ ID NO: 153, the HCDR3 sequence of SEQ ID NO: 157, the LCDR1 sequence of SEQ ID NO: 165, the LCDR2 sequence of SEQ ID NO: 169, and the LCDR3 sequence of SEQ ID NO: 173.

65. An antibody comprising any one of (1) to (12):
(1) a VH comprising the amino acid sequence of SEQ ID NO: 1 and a VL comprising the amino acid sequence of SEQ ID NO: 49;
(2) a VH comprising the amino acid sequence of SEQ ID NO: 2 and a VL comprising the amino acid sequence of SEQ ID NO: 50;
(3) a VH comprising the amino acid sequence of SEQ ID NO: 3 and a VL comprising the amino acid sequence of SEQ ID NO: 51;
(4) a VH comprising the amino acid sequence of SEQ ID NO: 4 and a VL comprising the amino acid sequence of SEQ ID NO: 52;
(5) a VH comprising the amino acid sequence of SEQ ID NO: 5 and a VL comprising the amino acid sequence of SEQ ID NO: 53;
(6) a VH comprising the amino acid sequence of SEQ ID NO: 6 and a VL comprising the amino acid sequence of SEQ ID NO: 54;
(7) a VH comprising the amino acid sequence of SEQ ID NO: 7 and a VL comprising the amino acid sequence of SEQ ID NO: 55;
(8) a VH comprising the amino acid sequence of SEQ ID NO: 8 and a VL comprising the amino acid sequence of SEQ ID NO: 56;
(9) a VH comprising the amino acid sequence of SEQ ID NO: 142 and a VL comprising the amino acid sequence of SEQ ID NO: 158;
(10) a VH comprising the amino acid sequence of SEQ ID NO: 143 and a VL comprising the amino acid sequence of SEQ ID NO:159;
(11) a VH comprising the amino acid sequence of SEQ ID NO: 144 and a VL comprising the amino acid sequence of SEQ ID NO: 160;
(12) a VH comprising the amino acid sequence of SEQ ID NO: 145 and a VL comprising the amino acid sequence of SEQ ID NO: 161.
